SEQUENCE LISTING (3 sequences)

SEQ ID NO: 1 - DNA, 40 bp, Artificial sequence
  Synthetic polynucleotide
ccttcctcccgtgtcttgtaccttcccgtcaggaggaagg

SEQ ID NO: 2 - DNA, 40 bp, Artificial sequence; identical to SEQ ID NO: 1
  Synthetic polynucleotide
  misc_feature (1)..(1): 5′ TAMRA
  misc_feature (40)..(40): 3′ BHQ

SEQ ID NO: 3 - DNA, 10 bp, Artificial sequence
  Synthetic polynucleotide
gacgggaagg

(19) United States
(12) Patent Application Publication
(10) Pub. No.: US 2022/0098154 A1
(43) Pub. Date: Mar. 31, 2022

(54) ACETAMIDO DERIVATIVES AS DNA POLYMERASE THETA INHIBITORS

(71) Applicant: IDEAYA Biosciences, Inc., South San Francisco, CA (US)

(72) Inventors: Hilary Plake BECK, South San Francisco, CA (US); Michael DILLON, South San Francisco, CA (US); Brian JONES, South San Francisco, CA (US); Luisruben P. MARTINEZ, South San Francisco, CA (US); Zhonghua PEI, South San Francisco, CA (US)

(21) Appl. No.: 17/426,408

(22) PCT Filed: Jan. 29, 2020
(86) PCT No.: PCT/US2020/015661

§ 371 Date: Jul. 28, 2021

Related U.S. Application Data
(60) Provisional application No. 62/798,774, filed on Jan. 30, 2019.

(51) Int. Cl. C07D 213/85; C07D 213/64; C07D 213/74; C07C 237/04; C07D 215/54; C07D 221/18; C07D 217/26; C07C 255/58; C07D 239/42; C07D 405/12; C07D 401/12; C07D 409/12

(52) U.S. Cl. CPC C07D 213/85; C07D 213/64; C07D 213/74; C07C 237/04; C07D 215/54; C07D 409/12; C07D 217/26; C07C 255/58; C07D 239/42; C07D 405/12; C07D 401/12; C07D 221/18

(57) ABSTRACT

Disclosed herein are certain acetamido derivatives that are DNA Polymerase Theta (Polθ) inhibitors of Formula (I).

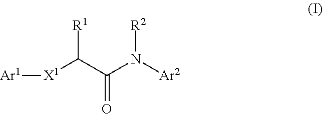

Also, disclosed are pharmaceutical compositions comprising such compounds and methods of treating diseases treatable by inhibition of Polθ such as cancer, including homologous recombination (HR) deficient cancers.

### CROSS-REFERENCES TO RELATED APPLICATIONS

This application claims the benefit under 35 U.S.C. 119(e) of U.S. Provisional Application No. 62/798,774, filed on Jan. 30, 2019, which is hereby incorporated herein by reference in its entirety for all purposes.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

This application contains a Sequence Listing which has been submitted electronically in ASCII format and is hereby incorporated by reference in its entirety. Said ASCII copy, created on Jan. 27, 2020, is named 052326-518WO_SL_ST25.txt and is 986 bytes in size.

### BACKGROUND

Targeting DNA repair deficiencies has become a proven and effective strategy in cancer treatment. However, DNA repair deficient cancers often become dependent on backup DNA repair pathways, which present an “Achilles heel” that can be targeted to eliminate cancer cells, and is the basis of synthetic lethality. Synthetic lethality is exemplified by the success of poly (ADP-ribose) polymerase (PARP) inhibitors in treating BRCA-deficient breast and ovarian cancers (Audeh M. W., et al., Lancet (2010); 376 (9737): 245-51).

DNA damage repair processes are critical for genome maintenance and stability, among which, double strand breaks (DSBs) are predominantly repaired by the nonhomologous end joining (NHEJ) pathway in G1 phase of the cell cycle and by homologous recombination (HR) in S-G2 phases. A less addressed alternative end-joining (alt-EJ), also known as microhomology-mediated end-joining (MMEJ) pathway, is commonly considered as a “backup” DSB repair pathway when NHEJ or HR are compromised. Numerous genetic studies have highlighted a role for polymerase theta (Polθ, encoded by POLQ) in stimulating MMEJ in higher organisms (see Chan S. H., et al., PLoS Genet. (2010); 6: e1001005; Roerink S. F., et al., Genome research. (2014); 24: 954-962; Ceccaldi R., et. al., Nature (2015); 518: 258-62; and Mateos-Gomez P. A., et al., Nature (2015); 518: 254-57).

The identification of mammalian POLQ initially arose from interest in the POLQ ortholog Mus308 gene product of Drosophila melanogaster. Mus308 mutants are hypersensitive to agents that cause DNA inter-strand cross-links (ICL) (Aguirrezabalaga I., et al., Genetics. (1995); 139:649-658), which implied that Mus308 may play a specific role in repair of ICLs in DNA. Characterization of the POLQ gene showed that it encodes an unusual domain configuration, with a large central portion flanking by a N-terminal DNA helicase domain and a C-terminal DNA polymerase domain (see Harris P. V., et al., Mol Cell Biol. (1996); 16: 5764-5771). The mechanisms by which Polθ polymerase functions in alt-EJ were also found to efficiently promote end-joining when overhangs contained >2 bp of microhomology were present (see Kent T., et al., Elife (2016); 5: e13740), and Kent T., et al., Nat. Struct. Mol. Biol. (2015); 22: 230-237. On the other hand, the helicase domain of Polθ contributes to microhomology annealing (see Chan S H et al., PLoS Genet. (2010); 6: e1001005; and Kawamura K et al., Int. J. Cancer (2004); 109: 9-16).

The expression of Polθ is largely absent in normal cells but upregulated in breast, lung, and ovarian cancers (see Ceccaldi R., et al., Nature (2015); 518, 258-62). Additionally, the increase of Polθ expression correlates with poor prognosis in breast cancer (see Lemee F et al., Proc Natl Acad Sci USA (2010); 107: 13390-5). It has been shown that cancer cells with deficiency in HR, NHEJ or ATM are highly dependent on Polθ expression (see Ceccaldi R., et al., Nature (2015); 518: 258-62, Mateos-Gomez P A et al., Nature (2015); 518: 254-57, and Wyatt D. W., et al., Mol. Cell (2016); 63: 662-73). Therefore, Polθ is an attractive target for novel synthetic lethal therapy in cancers containing DNA repair defects.

### SUMMARY

Disclosed herein are certain acetamido derivatives that are DNA Polymerase Theta (Polθ) inhibitors, in particular compounds that inhibit polymerase domain of Polθ. Also, disclosed are pharmaceutical compositions comprising such compounds and methods of treating and/or preventing diseases treatable by inhibition of Polθ such as cancer, including homologous recombination (HR) deficient cancers.

In a first aspect, provided is a compound of Formula (I):

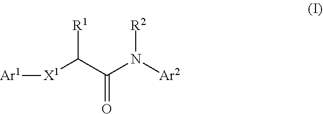

wherein:

X¹ is —NH— or —O—;

Ar¹ is phenyl or six- to ten-membered heteroaryl wherein phenyl and heteroaryl are substituted with R^{a} and further substituted with R^{b} and R^{c}, wherein R^{a} is haloalkyl and R^{b} and R^{c} are independently selected from hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R^{b} and/or R^{c} are unsubstituted or substituted with one, two, or three substituents independently selected from alkyl, halo, haloalkyl, and hydroxy;

R¹ is hydrogen, alkyl, hydroxyalkyl, alkoxyalkyl, aminoalkyl, aminocarbonylalkyl, or phenalkyl, wherein phenyl in phenalkyl is substituted with R^{d}, R^{e}, and R^{f}, wherein R^{d}, R^{e}, and R^{f} are independently selected from hydrogen, alkyl, halo, haloalkyl, alkoxy, haloalkoxy, hydroxy, and cyano;

R² is alkyl, deuteroalkyl, cycloalkyl, or haloalkyl;

Ar² is phenyl or heteroaryl wherein said phenyl and heteroaryl are substituted with R^{g}, R^{h}, and Rⁱ, wherein R^{g}, R^{h}, and Rⁱ are independently selected from hydrogen, alkyl, cycloalkyl, cycloalkyloxy, halo, haloalkyl, alkoxy, haloalkoxy, hydroxy, cyano, and —CONH₂; provided one of R^{g}, R^{h}, and Rⁱ is other than hydrogen; or

a pharmaceutically acceptable salt thereof, provided that:

(1) when X¹ is NH, R¹ is hydrogen, R² is methyl or ethyl, and Ar¹ is phenyl substituted with R^{a} and R^{b} where R^{a} is haloalkyl and R^{b} is hydrogen, chloro, methyl, or piperidin-1-yl, then Ar² is not 3-methylphenyl;

and

(2) the compound of Formula (I) is not:
Acetamide, N-(4-fluorophenyl)-N-methyl-2-[[5-(trifluoromethyl)-2-benzothiazolyl]oxy]-;
Acetamide, N-(5-bromo-2-pyridinyl)-N-ethyl-2-[3-(trifluoromethyl)phenoxy]-;
Acetamide, N-ethyl-N-(6-methoxy-3-pyridinyl)-2-[3-(trifluoromethyl)phenoxy]-;
Acetamide, N-ethyl-N-(4-fluorophenyl)-2-[3-(trifluoromethyl)phenoxy]-;
Acetamide, N-ethyl-N-(4-fluorophenyl)-2-[4-(trifluoromethyl)phenoxy]-;
Acetamide, N-(3,4-difluorophenyl)-N-ethyl-2-[4-(trifluoromethyl)phenoxy]-;
Acetamide, N-(3,4-difluorophenyl)-N-ethyl-2-[2-(trifluoromethyl)phenoxy]-;
Acetamide, N-(5-bromo-2-pyridinyl)-N-ethyl-2-[2-(trifluoromethyl)phenoxy]-;
Acetamide, N-(3,4-difluorophenyl)-N-ethyl-2-[3-(trifluoromethyl)phenoxy]-;
Acetamide, N-(4-bromo-2-methylphenyl)-N-methyl-2-[3-(trifluoromethyl)phenoxy]-;
Acetamide, N-(3-fluoro-4-methoxyphenyl)-N-(1-methylethyl)-2-[2-(trifluoromethyl)phenoxy]-;
Benzamide, 4-[methyl[2-[2-(trifluoromethyl)phenoxy]acetyl]amino]-;
Propanamide, 2-[2-chloro-4-(trifluoromethyl)phenoxy]-N-(4-fluorophenyl)-N-(1-methylethyl)-;
Acetamide, 2-[4-(bromomethyl)phenoxy]-N-(3-chlorophenyl)-N-methyl-;
Acetamide, N-ethyl-N-(4-fluorophenyl)-2-[2-(trifluoromethyl)phenoxy]-;
Acetamide, 2-[3,5-bis(trifluoromethyl)phenoxy]-N-(4-methyl-2-thiazolyl)-N-(2,2,2-trifluoroethyl)-;
Acetamido, 2-[3,5-bis(trifluoromethyl)phenoxy]-N-(2,6-difluorophenyl)-N-methyl-; or
a salt thereof.

In a second aspect, provided is a pharmaceutical composition comprising a compound of Formula (I) or a pharmaceutically acceptable thereof and at least one pharmaceutically acceptable excipient.

In a third aspect, provided is a method for treating and/or preventing a disease characterized by overexpression of Polθ in a patient comprising administering to the patient a therapeutically effective amount of:

(a) a compound of Formula (II′):

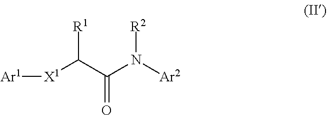

wherein:

X¹ is —NH— or —O—;

Ar¹ is aryl, six- to ten-membered heteroaryl, or fused heteroaryl wherein each of the aforementioned rings is substituted with R, R^{a}, R^{b}, and R^{c}, wherein R is hydrogen or halo, and R^{a}, R^{b} and R^{c} are independently selected from hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R^{a}, R^{b}, and R^{c} are unsubstituted or substituted with one, two, or three substituents independently selected from alkyl, halo, haloalkyl, and hydroxy;

R¹ is hydrogen, alkyl, hydroxyalkyl, alkoxyalkyl, aminoalkyl, aminocarbonylalkyl, or phenalkyl wherein phenyl in phenalkyl is substituted with R^{d}, R^{e}, and R^{f}, wherein R^{d}, R^{e}, and R^{f} are independently selected from hydrogen, alkyl, halo, haloalkyl, alkoxy, haloalkoxy, hydroxy, and cyano;

R² is alkyl, deuteroalkyl, cycloalkyl, or haloalkyl;

Ar² is phenyl, fused phenyl, or heteroaryl wherein said phenyl and heteroaryl are substituted with R^{g}, R^{h}, and Rⁱ, wherein R^{g}, R^{h}, and Rⁱ are independently selected from hydrogen, alkyl, cycloalkyl, cycloalkyloxy, halo, haloalkyl, alkoxy, haloalkoxy, hydroxy, cyano, alkylcarbonyl, and —CONH₂; or

(b) a compound of Formula (II):

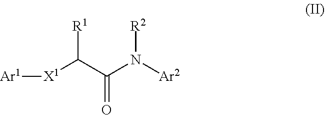

wherein:

X¹ is —NH— or —O—;

Ar¹ is aryl, six- to ten-membered heteroaryl, or fused heteroaryl wherein each of the aforementioned rings is substituted with R, R^{a}, R^{b}, and R^{c}, wherein R is hydrogen or halo, and R^{a}, R^{b} and R^{c} are independently selected from hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R^{a}, R^{b}, and R^{c} are unsubstituted or substituted with one, two, or three substituents independently selected from alkyl, halo, haloalkyl, and hydroxy;

R¹ is hydrogen, alkyl, hydroxyalkyl, alkoxyalkyl, aminoalkyl, aminocarbonylalkyl, or phenalkyl wherein phenyl in phenalkyl is substituted with R^{d}, R^{e}, and R^{f}, wherein R^{d}, R^{e}, and R^{f} are independently selected from hydrogen, alkyl, halo, haloalkyl, alkoxy, haloalkoxy, hydroxy, and cyano;

R² is alkyl, deuteroalkyl, cycloalkyl, or haloalkyl;

Ar² is phenyl or heteroaryl wherein said phenyl and heteroaryl are substituted with R^{g}, R^{h}, and Rⁱ, wherein R^{g}, R^{h}, and Rⁱ are independently selected from hydrogen, alkyl, cycloalkyl, cycloalkyloxy, halo, haloalkyl, alkoxy, haloalkoxy, hydroxy, cyano, and —CONH₂; or

(c) a compound of Formula (I) as defined in the first aspect; or

a pharmaceutically acceptable salt thereof (or an embodiment thereof disclosed herein).

In first embodiment of the third aspect, the patient is in recognized need of such treatment. In second embodiment of the third aspect and first embodiment contained therein, the compound of Formula (I), (II′) or (II) (or an embodiment thereof disclosed herein), or a pharmaceutically acceptable salt thereof is administered in a pharmaceutical composition. In third embodiment of the third aspect and first and second embodiments contained therein, the disease is a cancer.

In a fourth aspect, provided is a method of treating and/or preventing a homologous recombinant (HR) deficient cancer in a patient comprising administering to the patient a therapeutically effective amount of a compound of Formula (I), (II′) or (II) (or an embodiment thereof disclosed herein), or a pharmaceutically acceptable salt thereof. In first embodiment of the fourth aspect, the patient is in recognized need of such treatment. In second embodiment of the fourth aspect and first embodiment contained therein, the compound of Formula (I), (II′) or (II) (or an embodiment thereof disclosed herein), or a pharmaceutically acceptable salt thereof is administered in a pharmaceutical composition.

In a fifth aspect, provided is a method for inhibiting DNA repair by Polθ in a cancer cell comprising contacting the cell with an effective amount of a compound of Formula (I), (II′) or (II) (or an embodiment thereof disclosed herein), or a pharmaceutically acceptable salt thereof. In a first embodiment, the cancer is HR deficient cancer.

In a sixth aspect, provided is a method for treating and/or preventing a cancer in a patient, wherein the cancer is characterized by a reduction or absence of BRCA gene expression, the absence of the BRAC gene, or reduced function of BRCA protein, comprising administering to the subject a therapeutically effective amount of a compound of Formula (I), (II′) or (II) (or an embodiment thereof disclosed herein), or a pharmaceutically acceptable salt thereof optionally in a pharmaceutical composition.

In a seventh aspect, provided is a compound of Formula (I), (II′) or (II) (or an embodiment thereof disclosed herein), or a pharmaceutically acceptable salt thereof for inhibiting DNA repair by Polθ in a cell. In a first embodiment, the cell is HR deficient cell.

In an eighth aspect, provided is a compound of Formula (I), (II′) or (II) (or an embodiment thereof disclosed herein), or a pharmaceutically acceptable salt thereof for use in the treatment and/or prevention of a disease in a patient, wherein the disease is characterized by overexpression of Polθ.

In a ninth aspect, provided is a compound of Formula (I), (II′) or (II) (or an embodiment thereof disclosed herein), or a pharmaceutically acceptable salt thereof for use in the treatment and/or prevention of a cancer in a patient, wherein the cancer is characterized by a reduction or absence of BRAC gene expression, the absence of the BRAC gene, or reduced function of BRAC protein.

In a tenth aspect, provided is a compound of Formula (I), (II′) or (II) (or an embodiment thereof disclosed herein), or a pharmaceutically acceptable salt thereof for use in the treatment and/or prevention of a HR deficient cancer in a patient.

In an eleventh aspect, provided is a compound of Formula (I), (II′) or (II) (or any embodiment thereof disclosed herein), or a pharmaceutically acceptable salt thereof for use in the treatment or prevention of a cancer that is resistant to poly(ADP-ribose)polymerase (PARP) inhibitor therapy in a patient. Examples of cancers that are resistant to PARP-inhibitors include, but are not limited to, breast cancer, ovarian cancer, lung cancer, bladder cancer, liver cancer, head and neck cancer, pancreatic cancer, gastrointestinal cancer and colorectal cancer.

In any of the third to eleventh aspect, the cancer is lymphoma, soft tissue, rhabdoid, multiple myeloma, uterus, gastric, peripheral nervous system, rhabdomyosarcoma, bone, colorectal, mesothelioma, breast, ovarian, lung, fibroblast, central nervous system, urinary tract, upper aerodigestive, leukemia, kidney, skin, esophagus, and pancreas (data from large scale drop out screens in cancer cell lines indicate that some cell lines from the above cancers are dependent on polymerase theta for proliferation see https://depmap.org/portal/).

In first embodiment, a HR-deficient cancer is breast cancer. Breast cancer includes, but is not limited to, lobular carcinoma in situ, a ductal carcinoma in situ, an invasive ductal carcinoma, triple negative, HER positive, estrogen receptor positive, progesterone receptor positive, HER and estrogen receptor positive, HER and estrogen and progesterone receptor, positive inflammatory breast cancer, Paget disease of nipple, Phyllodes tumor, angiosarcoma, adenoid cystic carcinoma, low-grade adenosquamous carcinoma, medullary carcinoma, mucinous carcinoma, papillary carcinoma, tubular carcinoma, metaplastic carcinoma, micropapillary carcinoma, and mixed carcinoma. In second embodiment, HR-deficient cancer is ovarian cancer. Ovarian can includes, but is not limited to, epithelial ovarian carcinomas, maturing teratomas, dysgerminomas, endodermal sinus tumors, granulosa-theca tumors, Sertoli-Leydig cell tumors, and primary peritoneal carcinoma.

In a twelfth aspect, provided herein is a method of identifying Polθ polymerase domain inhibitory activity in a test compound, said method comprising
(i) contacting the test compound and Polθ polymerase domain (residues 1819-2590) in an assay buffer to form a reaction pre-mixture;
(ii) contacting the reaction pre-mixture of (i) with (a) a dNTP substrate mixture, and (b) a primed molecular beacon DNA to form a test solution,
   wherein the primed molecular beacon DNA comprises a labeled template annealed to a primer, wherein the labeled template is SEQ ID NO: 1 (5′-CCTTCCTCCCGTGTCTTGTACCTTCCCGTCAGGAGGAAGG-3′) having one or more fluorescent labels, and the primer is SEQ ID NO: 3 (5′-GACGGGAAGG-3′); and
(iii) measuring fluorescence intensity of the test reaction mixture, wherein said method further comprises performing steps (i)-(iii) with a positive control sample represented by Formula (I) or (II′) (or any embodiments thereof).

In some embodiments, the final concentration of Polθ polymerase domain in the test reaction mixture is 4 nM.

In some embodiments, the assay buffer is 20 m M TRIS, pH 7.80, 50 mM KCl, 10 mM MgCl₂, 1 mM DTT, 0.01% BSA, 0.01% Tween20.

In some embodiments, the dNTP substrate mixture is an equal mixture of each natural dNTP (dTTP, dATP, dCTP, and dGTP). In some embodiments the dNTP in the substrate mixture is 48 μM.

In some embodiments the labeled template is fluorescently labeled with one or more fluorescent labels. A number of fluorescent labels (and quenchers) are known in the art. In some embodiments the one or more fluorescent labels comprise 5′-TAMRA and 3′-BHQ. In some embodiments the sequence of the labeled template is SEQ ID NO 2: 5′-CCTTCCTCCCGTGTCTTGTACCTTCCCGTCAGGAGGAAGG-3′ with 5′-TAMRA and 3′-BHQ.

In some embodiments the primed molecular beacon DNA further comprises a priming buffer. In some embodiments, the buffer is 10 mM Tris-HCl pH 8.0, 100 mM NaCl buffer, and the concentration of the primed molecular beacon DNA is 96 nM.

A person of skill in the art will recognize that the fluorescence measured will depend on the labels being used in the assay. In some embodiments, absorbance (λₑₓ=485 nm, λₑₘ=535 nm) of the Polθ theta reaction mixture.

### BRIEF DESCRIPTION OF THE DRAWINGS

Not Applicable

### DETAILED DESCRIPTION

Before the present invention is further described, it is to be understood that the invention is not limited to the particular embodiments set forth herein, and it is also to be understood that the terminology used herein is for the purpose of describing particular embodiments only, and is not intended to be limiting.

The singular forms “a,” “an,” and “the” as used herein and in the appended claims include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

When needed, any definition herein may be used in combination with any other definition to describe a composite structural group. By convention, the trailing element of any such definition is that which attaches to the parent moiety. For example, the composite group alkoxyalkyl means that an alkoxy group is attached to the parent molecule through an alkyl group.

The publications discussed herein are provided solely for their disclosure prior to the filing date of the present application. Further, the dates of publication provided may be different from the actual publication dates, which may need to be independently confirmed.

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with one alkoxy group, as defined above, e.g., 2-methoxyethyl, 1-, 2-, or 3-methoxypropyl, 2-ethoxyethyl, and the like.

“Alkylcarbonyl” means a —C(O)R radical where R is alkyl as defined herein, e.g., methylcarbonyl, ethylcarbonyl, and the like.

“Amino” means a —NH₂.

“Alkylamino” means a —NHR radical where R is alkyl as defined above, e.g., methylamino, ethylamino, propylamino, or 2-propylamino, and the like.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen, alkyl, haloalkyl, hydroxyalkyl, alkoxyalkyl, or alkylcarbonyl, each as defined herein, e.g., aminomethyl, aminoethyl, methylaminomethyl, and the like.

“Aminocarbonylalkyl” means a -(alkylene)-CONH₂ radical wherein alkylene as defined herein, e.g., aminocarbonylmethyl, aminocarbonylethyl, aminocarbonylethyl, and the like. When the group is —CH₂CONH₂, it may be referred to herein as aminocarbonylmethyl.

“Aryl” means a monovalent monocyclic or bicyclic aromatic hydrocarbon radical of 6 to 10 ring atoms e.g., phenyl or naphthyl.

“Phenalkyl” means a -(alkylene)-R radical where R is phenyl e.g., benzyl, phenethyl, and the like.

“Cycloalkyl” means a monocyclic monovalent hydrocarbon radical of three to six carbon atoms which may be saturated or contains one double bond. Cycloalkyl may be unsubstituted or substituted with one or two substituents independently selected from alkyl, halo, alkoxy, hydroxy, or cyano. Examples include, but are not limited to, cyclopropyl, cyclobutyl, cyclopentyl, cyclohexyl, 1-cyanocycloprop-1-yl, 1-cyanomethylcycloprop-1-yl, 3-fluorocyclohexyl, and the like. When cycloalkyl contains a double bond, it may be referred to herein as cycloalkenyl.

“Cycloalkyloxy” means —O—R radical where R is cycloalkyl as defined above. Examples include, but are not limited to, cyclopropyloxy, cyclobutyloxy, and the like.

“Deuteroalkyl” means an alkyl radical as defined above wherein one to six hydrogen atoms in the alkyl radical are replaced by deuterium, e.g., -CD₃, —CH₂CD₃, and the like.

“Fused heteroaryl” means a six-membered heteroaryl ring fused to a three to six membered saturated cycloalkyl, each ring as defined herein.

“Fused phenyl” means phenyl fused to a four to six membered saturated heterocyclyl, each ring as defined herein.

“Halo” means fluoro, chloro, bromo, or iodo, preferably fluoro or chloro.

“Haloalkyl” means alkyl radical as defined above, which is substituted with one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to in this Application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon, unless stated otherwise. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heterocyclyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, azetidinyl, oxetanyl, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic.

“Oxo,” as used herein, alone or in combination, refers to ═(O).

“Pharmaceutically acceptable salts” as used herein is meant to include salts of the active compounds which are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of salts derived from pharmaceutically acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogen carbonic, phosphoric, monohydrogen phosphoric, dihydrogen phosphoric, sulfuric, monohydrogen sulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The present disclosure also includes protected derivatives of compounds of the present disclosure. For example, when compounds of the present disclosure contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, 5^{th} Ed., John Wiley & Sons, Inc. (2014), the disclosure of which is incorporated herein by reference in its entirety. The protected derivatives of compounds of the present disclosure can be prepared by methods well known in the art.

The present disclosure also includes prodrugs of the compound of Formula (I) or (II) (and any embodiment thereof disclosed herein including specific compounds) or a pharmaceutically acceptable salt thereof. Prodrugs of the compounds described herein are those compounds that readily undergo chemical changes under physiological conditions to provide the compounds of the present invention. An example, without limitation, of a prodrug would be a compound which is administered as an ester (the “prodrug”), but then is metabolically hydrolyzed to the carboxylic acid, the active entity. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of Formulae (I) and (II) (and any embodiment thereof disclosed herein including specific compounds) can exist in unsolvated forms as well as solvated forms, including hydrated forms. In general, the solvated forms are equivalent to unsolvated forms and are intended to be encompassed within the scope of the present invention. Certain compounds of Formulae (I) and (II) may exist in multiple crystalline or amorphous forms. In general, all physical forms are equivalent for the uses contemplated by the present disclosure and are intended to be within the scope of the present disclosure.

Certain compounds of Formulae (I) and (II) (and any embodiment thereof disclosed herein including specific compounds) possess asymmetric carbon atoms (optical centers) or double bonds; the racemates, diastereomers, geometric isomers, regioisomers and individual isomers (e.g., separate enantiomers) are all intended to be encompassed within the scope of the present invention. When a stereochemical depiction is shown, it is meant to refer the compound in which one of the isomers is present and substantially free of the other isomer. ‘Substantially free of’ another isomer indicates at least an 80/20 ratio of the two isomers, more preferably 90/10, or 95/5 or more. In some embodiments, one of the isomers will be present in an amount of at least 99%.

The compounds of Formulae (I) and (II) (and any embodiment thereof disclosed herein including specific compounds) may also contain unnatural amounts of isotopes at one or more of the atoms that constitute such compounds. Unnatural amounts of an isotope may be defined as ranging from the amount found in nature to an amount 100% of the atom in question. Exemplary isotopes that can be incorporated into compounds of the present invention, such as a compound of Formula (I) and (II) (and any embodiment thereof disclosed herein including specific compounds) include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds disclosed herein, including in Table 1 below one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

“Pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

“Disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Patient” is generally synonymous with the term “subject” and as used herein includes all mammals including humans. Examples of patients include humans, livestock such as cows, goats, sheep, pigs, and rabbits, and companion animals such as dogs, cats, rabbits, and horses. Preferably, the patient is a human.

“In need of treatment” as used herein means the patient is being treated by a physician or other caregiver after diagnoses of the disease. For example, the patient has been diagnosed as having a disease linked to overexpression of Polθ or a homologous recombination (HR)-deficient cancer.

“Administration”, “administer” and the like, as they apply to, for example, a patient, cell, tissue, organ, or biological fluid, refer to contact of, for example, a compound of Formula (I), a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Therapeutically effective amount” as used herein means the amount of a compound of Formula (I), (II′) or (II) (and any embodiment thereof disclosed herein including specific compounds) or a pharmaceutically acceptable salt thereof that, when administered to a patient for treating a disease either alone or as part of a pharmaceutical composition and either in a single dose or as part of a series of doses, is sufficient to affect such treatment for the disease. The “therapeutically effective amount” will vary depending on the compound, the disease and its severity and the age, weight, etc., of the mammal to be treated. The therapeutically effective amount can be ascertained by measuring relevant physiological effects, and it can be adjusted in connection with the dosing regimen and diagnostic analysis of the subject's condition, and the like. By way of example, measurement of the serum level of a compound of Formula (I) (or, e.g., a metabolite thereof) at a particular time post-administration may be indicative of whether a therapeutically effective amount has been used.

“Treating” or “treatment” of a disease includes:

(1) inhibiting the disease, i.e., arresting or reducing the development of the disease or its clinical symptoms; or

(2) relieving the disease, i.e., causing regression of the disease or its clinical symptoms.

“Inhibiting”, “reducing,” or any variation of these terms in relation of Polθ, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of Polθ activity compared to its normal activity.

The term “preventing” refers to causing the clinical symptoms of the disease not to develop in a mammal that may be exposed to or predisposed to the disease but does not yet experience or display symptoms of the disease.

The term “homologous recombination” refers to the cellular process of genetic recombination in which nucleotide sequences are exchanged between two similar or identical DNA.

The term “homologous recombination (HR) deficient cancer” refers to a cancer that is characterized by a reduction or absence of a functional HR repair pathway. HR deficiency may arise from absence of one or more HR-associated genes or presence of one or more mutations in one or more HR-associated genes. Examples of HR-associated genes include BRCA1 BRCA2, RAD54, RAD51B, Ct1P (Choline Transporter-Like Protein), PALB2 (Partner and Localizer of BRCA2), XRCC2 (X-ray repair complementing defective repair in Chinese hamster cells 2), RECQL4 (RecQ Protein-like 4), BLM (Bloom syndrome, RecQ helicase-like), WRN (Werner syndrome, one or more HR-associated genes), Nbs 1 (Nibrin), and genes coding Fanconi anemia (FA) proteins or FA like genes e.g., FANCA, FANCB, FANCC, FANCD1 (BRCA2), FANCD2, FANCE, FANCF, FANCG, FANCI, FANJ (BRIP1), FANCL, FANCM, FANCN (RALB2), FANCP (SLX4), FANCS (BRCA1), RAD51C and XPF.

The term “Polθ overexpression” refers to the increased expression or activity of Polθ enzyme in a diseased cell e.g., cancer cell, relative to expression or activity of Polθ enzyme in a control cell (e.g., non-diseased cell of the same type). The amount of The amount of Polθ overexpression can be at least 2-fold, at least 3-fold, at least 4-fold, at least 5-fold, Polθ overexpression can be at least 2-fold, at least 3-fold, at least 4-fold, at least 5-fold, at least 6-fold, at least 10-fold, at least 20-fold, at least 50-fold, relative to Polθ expression in a control cell. Examples of Polθ overexpressing cancers include, but are not limited to, certain ovarian, breast, cervical, lung, colorectal, gastric, bladder, and prostate cancers.

Representative compound of Formula (I) and (II) are listed in Table 1 below:

| Cpd. | | |
|---|---|---|
| No. | Structure | Name |
| 1 | 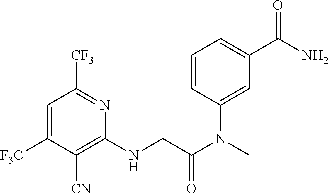 [SMILES: CN(C(=O)CNc1nc(C(F)(F)F)cc(C(F)(F)F)c1C#N)c1cccc(C(N)=O)c1] | 3-(2-(3-cyano-4,6-bis(trifluoromethyl)pyridin-2-yl- amino)-N-methylacetamido)benzamide |
| 2 | 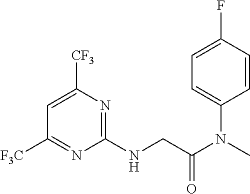 [SMILES: CN(C(=O)CNc1nc(C(F)(F)F)cc(C(F)(F)F)n1)c1ccc(F)cc1] | 2-(4,6-bis(trifluoromethyl)pyrimidin-2-ylamino)-N- (4-fluoro-phenyl)-N-methylacetamide |
| 3 | 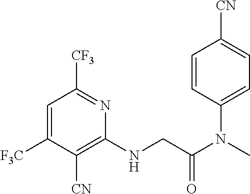 [SMILES: CN(C(=O)CNc1nc(C(F)(F)F)cc(C(F)(F)F)c1C#N)c1ccc(C#N)cc1] | 2-((3-cyano-4,6-bis(trifluoromethyl)pyridin-2-yl)- amino)-N-(4-cyanophenyl)-N-methylacetamide |
| 4 | 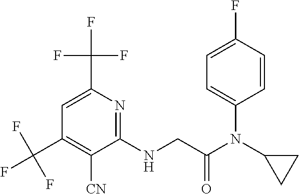 [SMILES: N#Cc1c(C(F)(F)F)cc(C(F)(F)F)nc1NCC(=O)N(c1ccc(F)cc1)C1CC1] | 2-((3-cyano-4,6-bis(trifluoromethyl)pyridin-2-yl)- amino)-N-cyclopropyl-N-(4- fluorophenyl)acetamide |
| 5 | 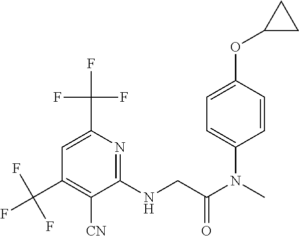 [SMILES: CN(C(=O)CNc1nc(C(F)(F)F)cc(C(F)(F)F)c1C#N)c1ccc(OC2CC2)cc1] | 2-((3-cyano-4,6-bis(trifluoromethyl)pyridin-2-yl)- amino)-N-(4-cyclopropoxyphenyl)-N-methyl- acetamide |
| 6 | 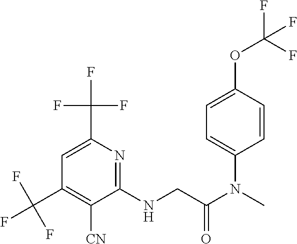 [SMILES: CN(C(=O)CNc1nc(C(F)(F)F)cc(C(F)(F)F)c1C#N)c1ccc(OC(F)(F)F)cc1] | 2-((3-cyano-4,6-bis(trifluoromethyl)pyridin-2-yl)- amino)-N-methyl-N-(4-(trifluoromethoxy)phenyl)- acetamide |
| 7 | 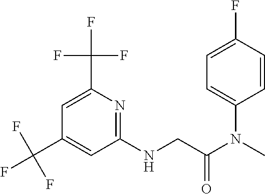 [SMILES: CN(C(=O)CNc1cc(C(F)(F)F)cc(C(F)(F)F)n1)c1ccc(F)cc1] | 2-((4,6-bis(trifluoromethyl)pyridin-2-yl)amino)-N- (4-fluoro-phenyl)-N-methylacetamide |
| 8 | 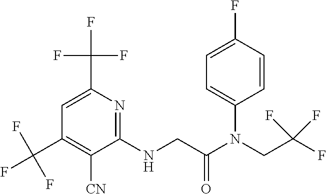 [SMILES: N#Cc1c(C(F)(F)F)cc(C(F)(F)F)nc1NCC(=O)N(CC(F)(F)F)c1ccc(F)cc1] | 2-(3-cyano-4,6-bis(trifluoromethyl)pyridin-2-yl- amino)-N-(4-fluorophenyl)-N-(2,2,2-trifluoroethyl)- acetamide |
| 9 | 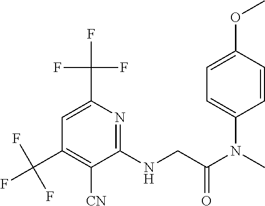 [SMILES: COc1ccc(N(C)C(=O)CNc2nc(C(F)(F)F)cc(C(F)(F)F)c2C#N)cc1] | 2-[[3-cyano-4,6-bis(trifluoromethyl)pyridin-2-yl]- amino]-N-(4-methoxyphenyl)-N-methylacetamide |
| 10 | 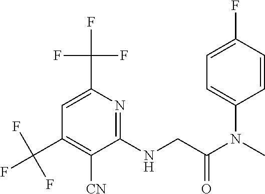 [SMILES: CN(C(=O)CNc1nc(C(F)(F)F)cc(C(F)(F)F)c1C#N)c1ccc(F)cc1] | 2-(3-cyano-4,6-bis(trifluoromethyl)pyridin-2-yl- amino)-N-(4-fluorophenyl)-N-methylacetamide |
| 11 | 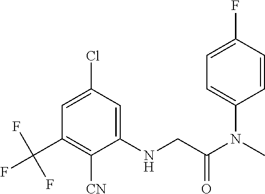 [SMILES: CN(C(=O)CNc1cc(Cl)cc(C(F)(F)F)c1C#N)c1ccc(F)cc1] | 2-[[5-chloro-2-cyano-3-(trifluoromethyl)phenyl]- amino]-N-(4-fluorophenyl)-N-methylacetamide |
| 12 | 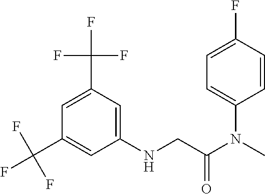 [SMILES: CN(C(=O)CNc1cc(C(F)(F)F)cc(C(F)(F)F)c1)c1ccc(F)cc1] | 2-((3,5-bis(trifluoromethyl)phenyl)amino)-N-(4- fluorophenyl)-N-methylacetamide |
| 13 | 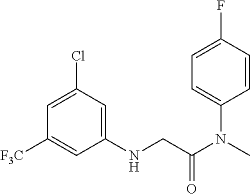 [SMILES: CN(C(=O)CNc1cc(Cl)cc(C(F)(F)F)c1)c1ccc(F)cc1] | 3-[[3-chloro-5-(trifluoromethyl)phenyl]amino]-N- (4-fluoro-phenyl)-N-methylacetamide |
| 14 | 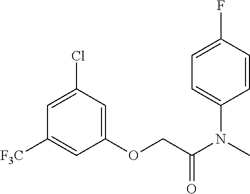 [SMILES: CN(C(=O)COc1cc(Cl)cc(C(F)(F)F)c1)c1ccc(F)cc1] | 2-[3-chloro-5-(trifluoromethyl)phenoxy]-N-(4- fluoro-phenyl)-N-methylacetamide |
| 15 | 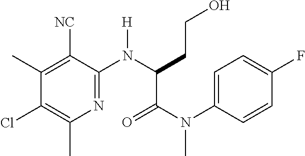 [SMILES: Cc1nc(N[C@@H](CCO)C(=O)N(C)c2ccc(F)cc2)c(C#N)c(C)c1Cl] | (S)-2-(5-chloro-3-cyano-4,6-dimethylpyridin-2-yl- amino)-N-(4-fluorophenyl)-4-hydroxy-N-methyl- butanamide |
| 16 | 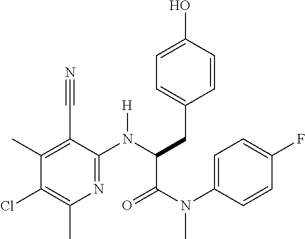 [SMILES: Cc1nc(N[C@@H](Cc2ccc(O)cc2)C(=O)N(C)c2ccc(F)cc2)c(C#N)c(C)c1Cl] | a mixture of S)-2-(5-chloro-3-cyano-4,6-dimethyl- pyridin-2-yl-amino)-N-(4-fluorophenyl)-3-(4- hydroxyphenyl)-N-methyl-propanamide and (R)-2-(5-chloro-3-cyano-4,6-dimethyl-pyridin-2- ylamino)-N-(4-fluorophenyl)-3-(4-hydroxyphenyl)- N-methyl-propanamide |
| | and | |
| | 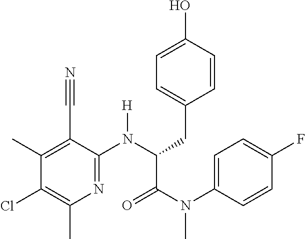 [SMILES: Cc1nc(N[C@H](Cc2ccc(O)cc2)C(=O)N(C)c2ccc(F)cc2)c(C#N)c(C)c1Cl] | |
| 17 | 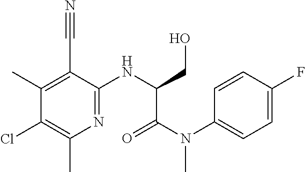 [SMILES: Cc1nc(N[C@@H](CO)C(=O)N(C)c2ccc(F)cc2)c(C#N)c(C)c1Cl] | (S)-2-amino-N-(4-fluorophenyl)-3-hydroxy-N- methylpropanamide |
| 18 | 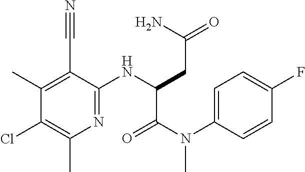 [SMILES: Cc1nc(N[C@@H](CC(N)=O)C(=O)N(C)c2ccc(F)cc2)c(C#N)c(C)c1Cl] | (S)-2-(5-chloro-3-cyano-4,6-dimethylpyridin-2-yl- amino)-N1-(4-fluorophenyl)-N1- methylsuccinamide |
| 19 | 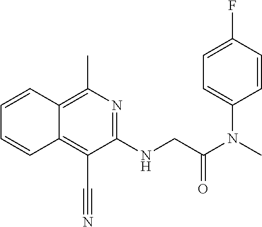 [SMILES: Cc1nc(NCC(=O)N(C)c2ccc(F)cc2)c(C#N)c2ccccc12] | 2-[(4-cyano-1-methylisoquinolin-3-yl)amino]-N-(4- fluorophenyl)-N-methylacetamide |
| 20 | 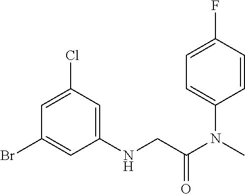 [SMILES: CN(C(=O)CNc1cc(Cl)cc(Br)c1)c1ccc(F)cc1] | 2-[(3-bromo-5-chlorophenyl)amino]-N-(4-fluoro- phenyl)-N-methylacetamide |
| 21 | 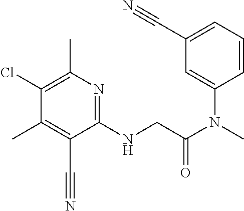 [SMILES: Cc1nc(NCC(=O)N(C)c2cccc(C#N)c2)c(C#N)c(C)c1Cl] | 2-((5-chloro-3-cyano-4,6-dimethylpyridin-2-yl)- amino)-N-(3-cyanophenyl)-N-methylacetamide |
| 22 | 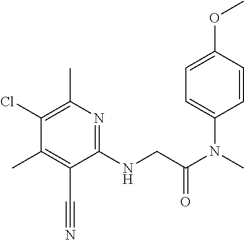 [SMILES: COc1ccc(N(C)C(=O)CNc2nc(C)c(Cl)c(C)c2C#N)cc1] | 2-(5-chloro-3-cyano-4,6-dimethylpyridin-2- ylamino)-N-(4-methoxyphenyl)-N-methylacetamide |
| 23 | 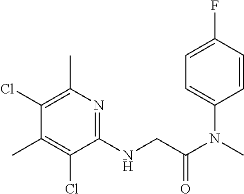 [SMILES: Cc1nc(NCC(=O)N(C)c2ccc(F)cc2)c(Cl)c(C)c1Cl] | 2-(3,5-dichloro-4,6-dimethylpyridin-2-ylamino)-N- (4-fluoro-phenyl)-N-methylacetamide |
| 24 | 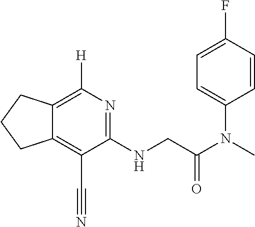 [SMILES: CN(C(=O)CNc1ncc2c(c1C#N)CCC2)c1ccc(F)cc1] | 2-(4-cyano-6,7-dihydro-5H-cyclopenta[c]pyridin-3- ylamino)-N-(4-fluorophenyl)-N-methylacetamide |
| 25 | 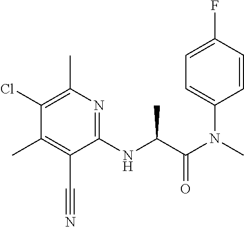 [SMILES: Cc1nc(N[C@@H](C)C(=O)N(C)c2ccc(F)cc2)c(C#N)c(C)c1Cl] | (S)-2-((5-chloro-3-cyano-4,6-dimethylpyridin-2- yl)-amino)-N-(4-fluorophenyl)-N- methylpropanamide |
| 26 | 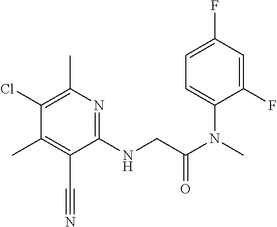 [SMILES: Cc1nc(NCC(=O)N(C)c2ccc(F)cc2F)c(C#N)c(C)c1Cl] | 2-((5-chloro-3-cyano-4,6-dimethylpyridin-2-yl)- amino)-N-(2,4-difluorophenyl)-N-methylacetamide |
| 27 | 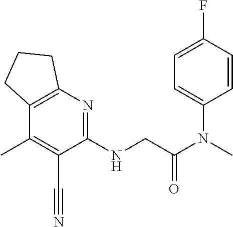 [SMILES: Cc1c(C#N)c(NCC(=O)N(C)c2ccc(F)cc2)nc2c1CCC2] | 2-([3-cyano-4-methyl-5H,6H,7H-cyclopenta[b]- pyridin-2-yl]amino)-N-(4-fluorophenyl)-N- methylacetamide |
| 28 | 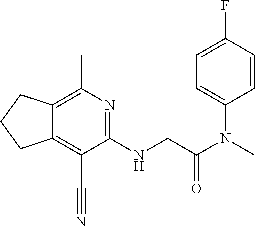 [SMILES: Cc1nc(NCC(=O)N(C)c2ccc(F)cc2)c(C#N)c2c1CCC2] | 2-(4-cyano-1-methyl-6,7-dihydro-5H- cyclopenta[c]-pyridin-3-ylamino)-N-(4- fluorophenyl)-N-methylacetamide |
| 29 | 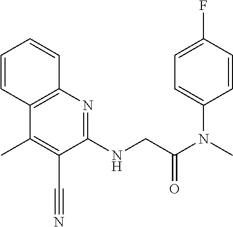 [SMILES: Cc1c(C#N)c(NCC(=O)N(C)c2ccc(F)cc2)nc2ccccc12] | 2-[(3-cyano-4-methylquinolin-2-yl)amino]-N-(4- fluorophenyl)-N-methylacetamide |
| 30 | 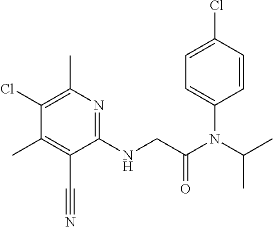 [SMILES: Cc1nc(NCC(=O)N(c2ccc(Cl)cc2)C(C)C)c(C#N)c(C)c1Cl] | 2-[(5-chloro-3-cyano-4,6-dimethylpyridin-2-yl)- amino]-N-(4-chlorophenyl)-N-(propan-2-yl)- acetamide |
| 31 | 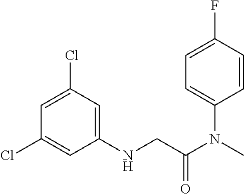 [SMILES: CN(C(=O)CNc1cc(Cl)cc(Cl)c1)c1ccc(F)cc1] | 2-[(3,5-dichlorophenyl)amino]-N-(4-fluorophenyl)- N-methyl-acetamide |
| 32 | 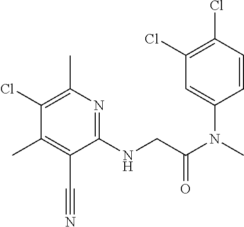 [SMILES: Cc1nc(NCC(=O)N(C)c2ccc(Cl)c(Cl)c2)c(C#N)c(C)c1Cl] | 2-(5-chloro-3-cyano-4,6-dimethylpyridin-2- ylamino)-N-(3,4-dichlorophenyl)-N- methylacetamide |
| 33 | 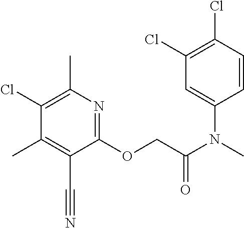 [SMILES: Cc1nc(OCC(=O)N(C)c2ccc(Cl)c(Cl)c2)c(C#N)c(C)c1Cl] | 2-(5-chloro-3-cyano-4,6-dimethylpyridin-2-yloxy)- N-(3,4-dichlorophenyl)-N-methylacetamide |
| 34 | 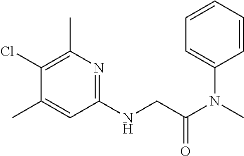 [SMILES: Cc1cc(NCC(=O)N(C)c2ccccc2)nc(C)c1Cl] | 2-[(5-chloro-4,6-dimethylpyridin-2-yl)amino]-N- methyl-N phenylacetamide |
| 35 | 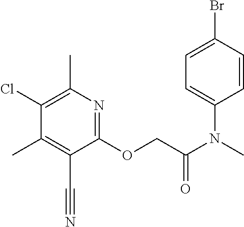 [SMILES: Cc1nc(OCC(=O)N(C)c2ccc(Br)cc2)c(C#N)c(C)c1Cl] | N-(4-bromophenyl)-2-[(5-chloro-3-cyano-4,6- dimethylpyridin-2-yl)oxy]-N-methylacetamide |
| 36 | 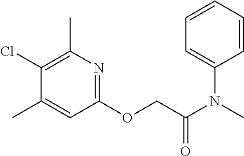 [SMILES: Cc1cc(OCC(=O)N(C)c2ccccc2)nc(C)c1Cl] | 2-(5-chloro-4,6-dimethylpyridin-2-yloxy)-N- methyl-N-phenyl-acetamide |
| 37 | 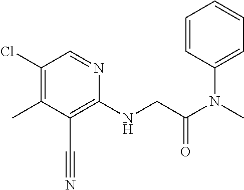 [SMILES: Cc1c(Cl)cnc(NCC(=O)N(C)c2ccccc2)c1C#N] | 2-(5-chloro-3-cyano-4-methylpyridin-2-ylamino)- N-methyl-N-phenylacetamide |
| 38 | 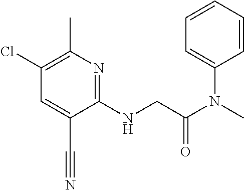 [SMILES: Cc1nc(NCC(=O)N(C)c2ccccc2)c(C#N)cc1Cl] | 2-(5-chloro-3-cyano-6-methylpyridin-2-ylamino)- N-methyl-N-phenylacetamide |
| 39 | 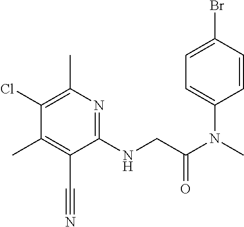 [SMILES: Cc1nc(NCC(=O)N(C)c2ccc(Br)cc2)c(C#N)c(C)c1Cl] | N-(4-bromophenyl)-2-(5-chloro-3-cyano-4,6- dimethyl-pyridin-2-ylamino)-N-methylacetamide |
| 40 | 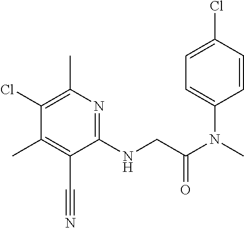 [SMILES: Cc1nc(NCC(=O)N(C)c2ccc(Cl)cc2)c(C#N)c(C)c1Cl] | 2-(5-chloro-3-cyano-4,6-dimethylpyridin-2- ylamino)-N-(4-chlorophenyl)-N-methylacetamide |
| 41 | 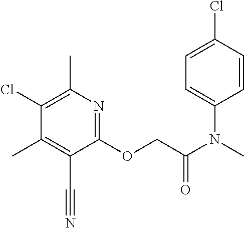 [SMILES: Cc1nc(OCC(=O)N(C)c2ccc(Cl)cc2)c(C#N)c(C)c1Cl] | 2-[(5-chloro-3-cyano-4,6-dimethylpyridin-2- yl)oxy]-N-(4-chloro-phenyl)-N-methylacetamide |
| 42 | 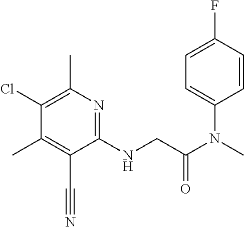 [SMILES: Cc1nc(NCC(=O)N(C)c2ccc(F)cc2)c(C#N)c(C)c1Cl] | 2-(5-chloro-3-cyano-4,6-dimethylpyridin-2- ylamino)-N-(4-fluorophenyl)-N-methylacetamide |
| 43 | 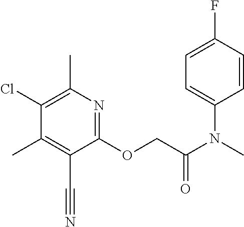 [SMILES: Cc1nc(OCC(=O)N(C)c2ccc(F)cc2)c(C#N)c(C)c1Cl] | 2-[(5-chloro-3-cyano-4,6-dimethylpyridin-2- yl)oxy]-N-(4-fluoro-phenyl)-N-methylacetamide |
| 44 | 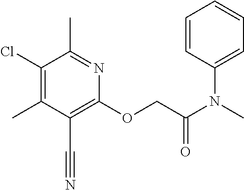 [SMILES: Cc1nc(OCC(=O)N(C)c2ccccc2)c(C#N)c(C)c1Cl] | 2-((5-chloro-3-cyano-4,6-dimethylpyridin-2- yl)oxy)-N-methyl-N-phenylacetamide |
| 45 | 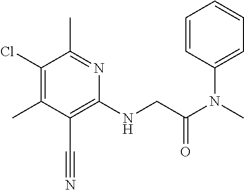 [SMILES: Cc1nc(NCC(=O)N(C)c2ccccc2)c(C#N)c(C)c1Cl] | 2-((5-chloro-3-cyano-4,6-dimethylpyridin-2- yl)amino)-N-methyl-N-phenylacetamide |
| 46 | 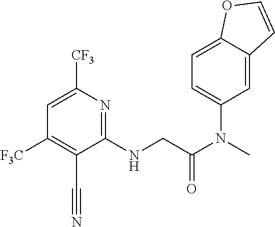 [SMILES: CN(C(=O)CNc1nc(C(F)(F)F)cc(C(F)(F)F)c1C#N)c1ccc2occc2c1] | N-(benzofuran-5-yl)-2-((3-cyano-4,6- bis(trifluoromethyl)pyridin-2-yl)amino)-N- methylacetamide |
| 47 | 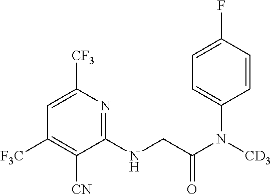 [SMILES: [2H]C([2H])([2H])N(C(=O)CNc1nc(C(F)(F)F)cc(C(F)(F)F)c1C#N)c1ccc(F)cc1] | 2-((3-cyano-4,6-bis(trifluoromethyl)pyridin-2-yl)- amino)-N-(4-fluorophenyl)-N-(methyl-d₃)acetamide |
| 48 | 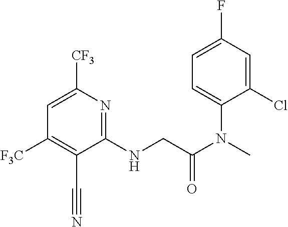 [SMILES: CN(C(=O)CNc1nc(C(F)(F)F)cc(C(F)(F)F)c1C#N)c1ccc(F)cc1Cl] | N-(2-chloro-4-fluorophenyl)-2-((3-cyano-4,6- bis(trifluoromethyl)pyridin-2-yl)amino)-N-methyl- acetamide |
| 49 | 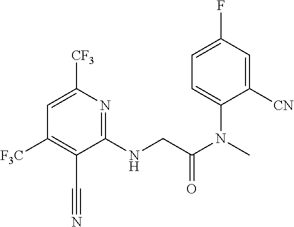 [SMILES: CN(C(=O)CNc1nc(C(F)(F)F)cc(C(F)(F)F)c1C#N)c1ccc(F)cc1C#N] | 2-((3-cyano-4,6-bis(trifluoromethyl)pyridin-2-yl)- amino)-N-(2-cyano-4-fluorophenyl)-N- methylacetamide |
| 50 | 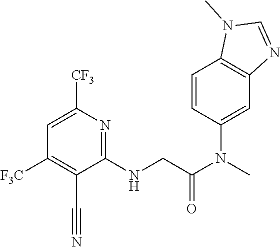 [SMILES: CN(C(=O)CNc1nc(C(F)(F)F)cc(C(F)(F)F)c1C#N)c1ccc2c(c1)ncn2C] | 2-((3-cyano-4,6-bis(trifluoromethyl)pyridin-2-yl)- amino)-N-methyl-N-(1-methyl-1H- benzo[d]imidazol-5-yl)acetamide |
| 51 | 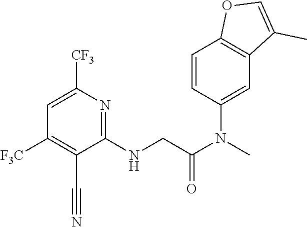 [SMILES: Cc1coc2ccc(N(C)C(=O)CNc3nc(C(F)(F)F)cc(C(F)(F)F)c3C#N)cc12] | 2-((3-cyano-4,6-bis(trifluoromethyl)pyridin-2-yl)- amino)-N-methyl-N-(3-methylbenzofuran-5-yl)- acetamide |
| 52 | 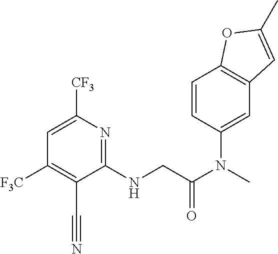 [SMILES: Cc1cc2cc(N(C)C(=O)CNc3nc(C(F)(F)F)cc(C(F)(F)F)c3C#N)ccc2o1] | 2-((3-cyano-4,6-bis(trifluoromethyl)pyridin-2-yl)- amino)-N-methyl-N-(2-methylbenzofuran-5-yl)- acetamide |
| 53 | 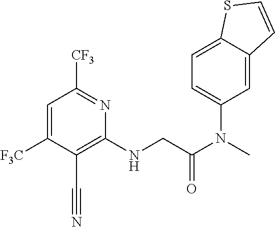 [SMILES: CN(C(=O)CNc1nc(C(F)(F)F)cc(C(F)(F)F)c1C#N)c1ccc2sccc2c1] | N-(benzo[b]thiophen-5-yl)-2-((3-cyano-4,6-bis- (trifluoromethyl)pyridin-2-yl)amino)-N-methyl- acetamide |
| 54 | 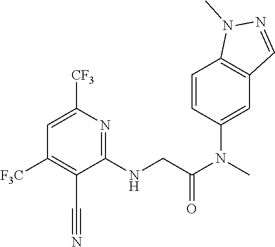 [SMILES: CN(C(=O)CNc1nc(C(F)(F)F)cc(C(F)(F)F)c1C#N)c1ccc2c(cnn2C)c1] | 2-((3-cyano-4,6-bis(trifluoromethyl)pyridin-2-yl)- amino)-N-methyl-N-(1-methyl-1H-indazol-5-yl)- acetamide |
| 55 | 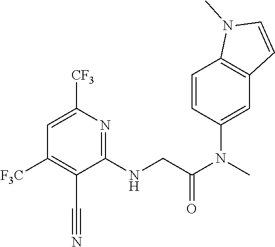 [SMILES: CN(C(=O)CNc1nc(C(F)(F)F)cc(C(F)(F)F)c1C#N)c1ccc2c(ccn2C)c1] | 2-((3-cyano-4,6-bis(trifluoromethyl)pyridin-2-yl)- amino)-N-methyl-N-(1-methyl-1H-indol-5- yl)acetamide |
| 56 | 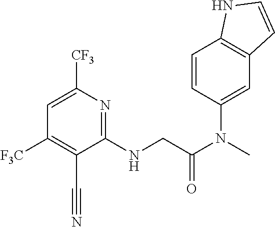 [SMILES: CN(C(=O)CNc1nc(C(F)(F)F)cc(C(F)(F)F)c1C#N)c1ccc2[nH]ccc2c1] | 2-((3-cyano-4,6-bis(trifluoromethyl)pyridin-2-yl)- amino)-N-(1H-indol-5-yl)-N-methylacetamide |
| 57 | 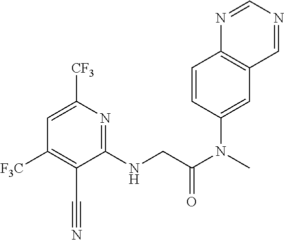 [SMILES: CN(C(=O)CNc1nc(C(F)(F)F)cc(C(F)(F)F)c1C#N)c1ccc2ncncc2c1] | 2-((3-cyano-4,6-bis(trifluoromethyl)pyridin-2-yl)- amino)-N-methyl-N-(quinazolin-6-yl)acetamide |
| 58 | 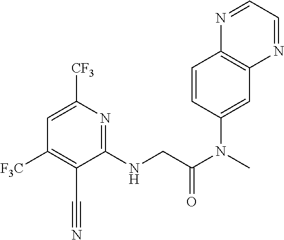 [SMILES: CN(C(=O)CNc1nc(C(F)(F)F)cc(C(F)(F)F)c1C#N)c1ccc2nccnc2c1] | of 2-((3-cyano-4,6-bis(trifluoromethyl)pyridin-2- yl)-amino)-N-methyl-N-(quinoxalin-6-yl)acetamide |
| 59 | 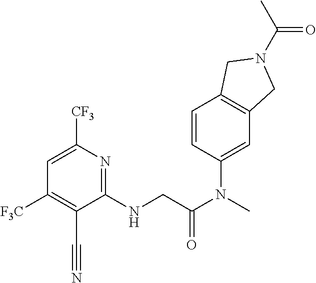 [SMILES: CC(=O)N1Cc2ccc(N(C)C(=O)CNc3nc(C(F)(F)F)cc(C(F)(F)F)c3C#N)cc2C1] | N-(2-acetylisoindolin-5-yl)-2-((3-cyano-4,6-bis- (trifluoromethyl)pyridin-2-yl)amino)-N-methyl- acetamide |
| 60 | 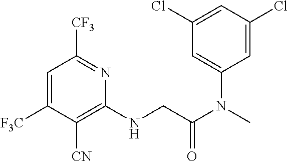 [SMILES: CN(C(=O)CNc1nc(C(F)(F)F)cc(C(F)(F)F)c1C#N)c1cc(Cl)cc(Cl)c1] | 2-((3-cyano-4,6-bis(trifluoromethyl)pyridin-2-yl)- amino)-N-(3,5-dichlorophenyl)-N-methylacetamide |
| 61 | 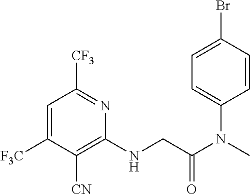 [SMILES: CN(C(=O)CNc1nc(C(F)(F)F)cc(C(F)(F)F)c1C#N)c1ccc(Br)cc1] | N-(4-bromophenyl)-2-((3-cyano-4,6-bis(trifluoro- methyl)pyridin-2-yl)amino)-N-methylacetamide |

### EMBODIMENTS

In embodiments 1 to 12 below, the present disclosure includes:

1. In embodiment 1, provided is a compound of Formula (I), or a pharmaceutically acceptable salt thereof, where R, R¹, X¹, Ar¹, and Ar² are as described in the Summary above.

2. In embodiment 2, provided is a compound of Formula (II), or a pharmaceutically acceptable salt thereof, where R, R¹, X¹, Ar¹, and Ar² are as described in the Summary above.

2A. In embodiment 2A, provided is a compound of Formula (II′), or a pharmaceutically acceptable salt thereof, where R, R¹, X¹, Ar¹, and Ar² are as described in the Summary above.

3. In embodiment 3, the compound of embodiment 1, 2 or 2A, or a pharmaceutically acceptable salt thereof, is wherein Ar¹ is a six- to ten-membered heteroaryl substituted with R^{a} where R^{a} is haloalkyl and further substituted with R^{b} and R^{c}.

4. In embodiment 4, the compound of embodiment 1, 2, or 2A, or a pharmaceutically acceptable salt thereof, is wherein Ar¹ is a six-membered heteroaryl substituted with R^{a}, where R^{a} is haloalkyl, and further substituted with R^{b} and R^{c}. In a first subembodiment of embodiment 4, Ar¹ is pyridinyl substituted with R^{a} and R^{b} and R^{c}. In a second subembodiment of embodiment 4, Ar¹ is pyridinyl substituted with R^{a}, where R^{a} is difluoromethyl or trifluoromethyl, and further substituted with R^{b} and R^{c}. In a third subembodiment of embodiment 4, Ar¹ is pyridin-2-yl substituted with R^{a}, where R^{a} is difluoromethyl or trifluoromethyl, and further substituted with R^{b} and/or R^{c}, where R^{b} is haloalkyl, alkoxy, halo, haloalkoxy, hydroxy, or cyano, and R^{c} is hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R^{c} are unsubstituted or substituted with one, two, or three substituents independently selected from alkyl, halo, haloalkyl, and hydroxy. In a fourth subembodiment of embodiment 4, Ar¹ is 4,6-di-trifluoromethylpyridin-2-yl, 3-cyano-4,6-di-trifluoromethylpyridin-2-yl, or 4,6-di-trifluoromethylpyrimidin-2-yl.

5. In embodiment 5, the compound of embodiment 1, 2 or 2A, or a pharmaceutically acceptable salt thereof, is wherein Ar¹ is phenyl substituted with R^{a}, where R^{a} is haloalkyl, and further substituted with R^{b} and R^{c}. In a first subembodiment of embodiment 5, Ar¹ is phenyl substituted with R^{a}, where R^{a} is difluoromethyl or trifluoromethyl, and further substituted with R^{b} and R^{c}. In a second subembodiment of embodiment 5, Ar¹ is phenyl substituted with R^{a}, where R^{a} is difluoromethyl or trifluoromethyl, and further substituted with R^{b} and/or R^{c}, where R^{b} is haloalkyl, alkoxy, halo, haloalkoxy, hydroxy, or cyano, and R^{c} is hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R^{c} are unsubstituted or substituted with one, two, or three substituents independently selected from alkyl, halo, haloalkyl, and hydroxy. In a fourth subembodiment of embodiment 5, Ar¹ is 3-chloro-5-trifluoromethylphenyl, 3-chloro-6-cyano-5-trifluoromethylphenyl, or 3,5-ditrifluoromethylphenyl.

6. In embodiment 6, the compound of embodiment 2 or 2A, or a pharmaceutically acceptable salt thereof, is wherein Ar¹ is phenyl, six- to ten-membered heteroaryl, or fused heteroaryl wherein each of the aforementioned rings are substituted with R, R^{a}, R^{b}, R^{c} and R^{d}. In a first subembodiment of embodiment 6, Ar¹ is phenyl substituted with R, R^{a}, R^{b}, R^{c} and R^{d}. In a second subembodiment of embodiment 6, Ar¹ is a six- to ten-membered heteroaryl substituted with R, R^{a}, R^{b}, R^{c} and R^{d}. In a third subembodiment of embodiment 6, Ar¹ is fused heteroaryl substituted with R, R^{a}, R^{b}, R^{c} and R^{d}. In a fourth subembodiment of embodiment 6, Ar¹ is 4-chloro-2-cyano-3,6-dimethylphenyl, 4-cyano-1-methylisoquinolin-3-yl, 3-bromo-5-chlorophenyl, 5-chloro-3-cyano-4,6-dimethylpyridin-2-yl, 3,5-dichloro-4,6-dimethylpyridin-2-yl, 4-cyano-6,7-dihydro-5H-cyclopenta[c]pyridin-2-yl, 3-cyano-4-methyl-6,7-dihydro-5H-cyclopenta[b]pyridin-2-yl, 4-cyano-1-methyl-6,7-dihydro-5H-cyclopenta-[c]pyridin-2-yl, 3-cyano-4-methylquinolin-2-yl, 3,5-dichlorophenyl, 5-chloro-4,6-dimethylpyridin-2-yl, 3-cyano-5-chloro-4-methylpyridin-2-yl, 3-cyano-5-chloro-6-methylpyridin-2-yl, or 3-cyano-5-chloro-4,6-dimethylpyridin-2-yl.

7. In embodiment 7, the compound of any one of embodiments 1 to 6 (and subembodiments therein), or a pharmaceutically acceptable salt thereof, is wherein R¹ is hydrogen, methyl, hydroxymethyl, 2-hydroxyethyl, 4-hydroxybenzyl, or aminocarbonylmethyl. In a first subembodiment of embodiment 7, R¹ is hydrogen.

8. In embodiment 8, the compound of any one of embodiments 1 to 7 (and subembodiments therein), or a pharmaceutically acceptable salt thereof, is wherein R² is alkyl, cycloalkyl, or haloalkyl. In a first subembodiment of embodiment 8, R² is methyl, ethyl, isopropyl, cyclopropyl, or 2,2,2-trifluoroethyl. In a second subembodiment of embodiment 8, R² is methyl.

9. In embodiment 9, the compound of any one of embodiments 1 to 8 (and subembodiments therein), or a pharmaceutically acceptable salt thereof, is wherein Ar² is phenyl, wherein said phenyl is substituted with R^{g}, R^{h}, and Rⁱ, wherein R^{g}, R^{h}, and Rⁱ are independently selected from hydrogen, alkyl, cycloalkyl, cycloalkyloxy, halo, haloalkyl, alkoxy, haloalkoxy, hydroxy, cyano, and —CONH₂. In a first subembodiment of embodiment 9, Ar² is phenyl substituted with R^{g}, R^{h}, and Rⁱ, wherein R^{g}, R^{h}, and Rⁱ are independently selected from hydrogen, —CONH₂, fluoro, chloro, bromo, cyano, methoxy, cyclopropyloxy, cyclobutyloxy, cyclopentyloxy, trifluoromethyl, or trifluoromethoxy. In a second subembodiment of embodiment 9, Ar¹ is phenyl, 4-fluorophenyl, 4-chlorophenyl, 4-bromophenyl, 3,4-dichlorophenyl, 2,4-difluorophenyl, 4-methoxyphenyl, 4-cyclopropoxyphenyl, 4-trifluoromethoxyphenyl, 3- or 4-CONH₂phenyl, or 4-cyanophenyl.

10. In embodiment 10, the compound of any one of embodiments 1 to 8 (and subembodiments therein), or a pharmaceutically acceptable salt thereof, is wherein Ar² is heteroaryl (e.g., benzofuranyl, benzimidazolyl, benzthiazolyl, indazolyl, indolyl, quinazolinyl, or quinoxalinyl) wherein said heteroaryl is substituted with R^{g}, R^{h}, and R, wherein R^{g}, R^{h}, and Rⁱ are independently selected from hydrogen, alkyl, cycloalkyl, cycloalkyloxy, halo, haloalkyl, alkoxy, haloalkoxy, hydroxy, cyano, and —CONH₂. In a first subembodiment of embodiment 10, R^{g}, R^{h}, and Rⁱ are independently selected from hydrogen, —CONH₂, fluoro, chloro, bromo, cyano, methoxy, cyclopropyloxy, cyclobutyloxy, cyclopentyloxy, trifluoromethyl, or trifluoromethoxy. In a first subembodiment of embodiment 10, Ar² is benzofuran-5-yl, quinoxalin-6-yl, quinazolin-6-yl, 1H-indol-5-yl, 1-methyl-indol-5-yl, 1-methyl-1H-indazol-5-yl, benzo[b]thiophen-5-yl, 3-methylbenzofuran-5-yl, or 1-methyl-1H-benzo[d]imidazol-5-yl.

11. In embodiment 11, the compound of any one of embodiments 2 to 8 (and subembodiments therein), or a pharmaceutically acceptable salt thereof, is wherein Ar² is fused phenyl wherein said fused phenyl is substituted with R^{g}, R^{h}, and Rᵢ, wherein R^{g}, R^{h}, and Rⁱ are independently selected from hydrogen, alkyl, cycloalkyl, cycloalkyloxy, halo, haloalkyl, alkoxy, haloalkoxy, hydroxy, cyano, alkylcarbonyl, and —CONH₂.

12. In embodiment 12, the compound of any one of embodiments 1 to 11 (and subembodiments therein), or a pharmaceutically acceptable salt thereof, is wherein X¹ is NH.

13. In embodiment 13, the compound of any one of embodiments 1 to 11 (and subembodiments therein), or a pharmaceutically acceptable salt thereof, is wherein X¹ is O.

It is understood that the embodiments set forth above include combinations of one or more of embodiments and/or subembodiments listed therein. For example, the Ar¹ group listed in embodiment 9 and subembodiment therein, can independently combine with one or more of the embodiments 1 to 8 and 10 to 13 and/or subembodiments contained therein.

The present disclosure includes further embodiments 14 to 30 below:

14. In embodiment 14, provided is a compound of Formula (I) disclosed in the Summary above.

15. In embodiment 15, the compound of embodiment 14, wherein Ar¹ is a six- to ten-membered heteroaryl substituted with R^{a} and further substituted with R^{b} and R^{c}.

16. In embodiment 16, the compound of embodiment 14, wherein Ar¹ is a six-membered heteroaryl substituted with R^{a} and further substituted with R^{b} and R^{c}.

17. In embodiment 17, the compound of embodiment 16, wherein Ar¹ is pyridinyl substituted with R^{a} and further substituted with R^{b} and R.

18. In embodiment 18, the compound of embodiment 16, wherein Ar¹ is pyridinyl substituted with R^{a}, where R^{a} is difluoromethyl or trifluoromethyl, and further substituted with R^{b} and R^{c}.

19. In embodiment 19, the compound of embodiment 16, wherein Ar¹ is pyridinyl substituted with R^{a}, where R^{a} is difluoromethyl or trifluoromethyl, and further substituted with R^{b} and/or R^{c}, where R^{b} is haloalkyl, alkoxy, halo, haloalkoxy, hydroxy, or cyano, and R is hydrogen, alkyl, halo, haloalkyl, alkoxy, haloalkoxy, hydroxy, cyano, cyanomethyl, aminocarbonylmethyl, heteroaryl, and heterocyclyl wherein said heteroaryl and heterocyclyl of R are unsubstituted or substituted with one, two, or three substituents independently selected from alkyl, halo, haloalkyl, and hydroxy.

20. In embodiment 20, the compound of embodiment 14, wherein Ar¹ is phenyl substituted with R^{a} and further substituted with R^{b} and R^{c}.

21. In embodiment 21, the compound of embodiment 20, wherein Ar¹ is phenyl substituted with R^{a}, where R^{a} is difluoromethyl or trifluoromethyl, and further substituted with R^{b} and R^{c}.

22. In embodiment 22, the compound of embodiment 20, wherein Ar¹ is phenyl substituted with R^{a} and R^{b} and/or R^{c}, where R^{a} is difluoromethyl or trifluoromethyl, R^{b} is haloalkyl, alkoxy, halo, haloalkoxy, hydroxy, or cyano, and R^{c} is hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R^{c} are unsubstituted or substituted with one, two, or three substituents independently selected from alkyl, halo, haloalkyl, and hydroxy.

23. In embodiment 23, the compound of any one of embodiments 14 to 22, wherein R¹ is hydrogen, methyl, hydroxymethyl, 2-hydroxyethyl, 4-hydroxybenzyl, or aminocarbonylethyl.

24. In embodiment 24, the compound of any one of embodiments 14 to 22 wherein R² is alkyl, cycloalkyl, or haloalkyl.

25. In embodiment 25, the compound of any of embodiments 14 to 22, wherein R¹ is hydrogen and R² is methyl, ethyl, isopropyl, cyclopropyl, or 2,2,2-trifluoroethyl.

26. In embodiment 26, the compound of any of embodiments 14 to 25, wherein Ar² is phenyl, wherein said phenyl is substituted with R^{g}, R^{h}, and Rⁱ independently selected from hydrogen, alkyl, cycloalkyl, cycloalkyloxy, halo, haloalkyl, alkoxy, haloalkoxy, hydroxy, cyano, and —CONH₂.

27. In embodiment 27, the compound of any one of embodiments 14 to 25, wherein Ar² is phenyl substituted with R^{g}, R^{h}, and Rⁱ, wherein R^{g}, R^{h}, and Rⁱ are independently selected from hydrogen, —CONH₂, fluoro, chloro, bromo, cyano, methoxy, cyclopropyloxy, cyclobutyloxy, cyclopentyloxy, trifluoromethyl, or trifluoromethoxy.

28. In embodiment 28, the compound of any of embodiments 14 to 25, wherein Ar² is heteroaryl wherein said heteroaryl is substituted with R^{g}, R^{h}, and Rⁱ independently selected from hydrogen, alkyl, cycloalkyl, cycloalkyloxy, halo, haloalkyl, alkoxy, haloalkoxy, hydroxy, cyano, and —CONH₂.

29. In embodiment 29, the compound of any of embodiments 14 to 28, wherein X¹ is NH.

30. In embodiment 30, the compound of any of embodiments 14 to 28, wherein X¹ is O.

### General Synthetic Schemes

Compounds of this disclosure can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., such as from about 0° C. to about 125° C. and further such as at about room (or ambient) temperature, e.g., about 20° C.

Compounds of Formula (I) and (II) where X¹ is NH and other groups are as defined in the Summary can be prepared the method illustrated and described in Scheme 1 below.

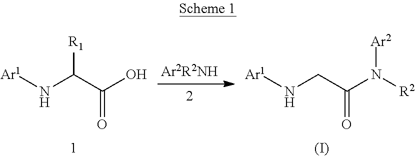

Reaction of an amino acid derivative of formula 1 where Ar¹ and R¹ are as defined in the Summary with an amine of formula 2 where Ar² is defined in the Summary under amino acid coupling reaction conditions known in the art provides a compound of Formula (I). Compounds of formula 1 are commercially available or can be prepared by methods well known in the art. For example, compound 1 can be prepared by reacting an amino acid of formula NH₂CHR¹CO₂H where R¹ is as defined in the Summary with an amine of formula Ar¹X where X is halo in the presence of a base or under Pd coupling reaction conditions known in the art.

Amino acids NH₂CHR¹CO₂H and amines of formula Ar¹X and formula 2 are commercially available or they can be prepared by methods well known in the art. For example, glycine, alanine, serine, phenylalanine, lysine, phenylalanine, and 2-amino-3-(hydroxyphenyl)-propionic, aniline, 2,5-dichloro-4,6-dimethylnicotinonitrile, 3,6-dichloro-2,4-dimethylpyridine, 3,5-dichloroaniline, 4-fluoro-N-methylaniline, 4-difluoro-N-methylaniline, 4-methoxy-N-methylaniline, 3-(methylamino)benzonitrile, N-methyl-4-(trifluoromethoxy)aniline, 4-cyclopropoxy-N-methylaniline, are commercially available.

Alternatively, compounds of Formula (I) and (II) where X¹ is NH and other groups are as defined in the Summary can be prepared the method illustrated and described in Scheme 2 below.

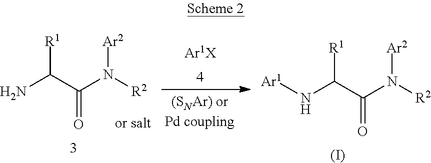

Compounds of Formula (I) and (II) can also be prepared by reacting an amide of formula 3 or it's salt with an arylhalide of formula 4 where Ar¹ is as defined in the Summary in the presence of a base such as N-methylpyridine, diethylisopropylamine, pyridine, and the like, or under Palladium reaction conditions well known in the art. Compounds of formula 3 can be prepared by reacting an amine of formula Ar¹NH₂ where Ar¹ is as defined in the Summary with an amino acid of formula PGNHCHR¹CO₂H where PG is a nitrogen protecting group such as Boc, Cbz and the like and R¹ is as defined in the Summary under amino acid coupling reaction conditions, followed by removal of the amino protecting group to provide a compound of formula 3.

### Assay

The ability of compounds of the disclosure to inhibit Polθ can be measured as described in Biological Example 1 below.

### Pharmaceutical Composition

The compounds of Formula (I), (II′), or (II), or a pharmaceutically acceptable salt thereof, provided herein may be in the form of compositions suitable for administration to a subject. In general, such compositions are pharmaceutical compositions comprising a compound of Formula (I), (II′), or (II) or a pharmaceutically acceptable salt thereof and one or more pharmaceutically acceptable or physiologically acceptable excipients. In certain embodiments, the compound of Formula (I), (II′), or (II), or a pharmaceutically acceptable salt thereof is present in a therapeutically effective amount. The pharmaceutical compositions may be used in the methods disclosed herein; thus, for example, the pharmaceutical compositions can be administered ex vivo or in vivo to a subject in order to practice the therapeutic methods and uses described herein.

The pharmaceutical compositions can be formulated to be compatible with the intended method or route of administration; exemplary routes of administration are set forth herein. Furthermore, the pharmaceutical compositions may be used in combination with other therapeutically active agents or compounds as described herein in order to treat the diseases, disorders and conditions contemplated by the present disclosure.

The pharmaceutical compositions containing the active ingredient (e.g., a compound of Formula (I), (II′), or (II), a pharmaceutically acceptable salt thereof) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets, capsules, and the like. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl di-stearate may be employed. The tablets may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinyl acetate, methylcellulose, carboxymethylcellulose, protamine sulfate, or lactide and glycolide copolymers, polylactide and glycolide copolymers, or ethylene vinyl acetate copolymers in order to control delivery of an administered composition. For example, the oral agent can be entrapped in microcapsules prepared by coacervation techniques or by interfacial polymerization, by the use of hydroxymethyl cellulose or gelatin-microcapsules or poly (methyl methacrylate) microcapsules, respectively, or in a colloid drug delivery system. Colloidal dispersion systems include macromolecule complexes, nanocapsules, microspheres, microbeads, and lipid-based systems, including oil-in-water emulsions, micelles, mixed micelles, and liposomes. Methods for the preparation of the above-mentioned formulations are known in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, (hydroxypropyl)methyl cellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., poly-oxyethylene stearate), or condensation products of ethylene oxide with long chain aliphatic alcohols (e.g., for heptdecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a compound of Formula (I), (II′), or (II), or a salt thereof, and one or more pharmaceutically acceptable excipient. Suitable pharmaceutically acceptable excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver a compound of Formula (I), (II′), or (II), or a salt thereof, including implants (e.g., implantable pumps) and catheter systems, slow injection pumps and devices, all of which are well known to the skilled artisan.

Depot injections, which are generally administered subcutaneously or intramuscularly, may also be utilized to release the compound of Formula (I), (II′), or (II), or a salt thereof disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. The suspension may be formulated according to the known art using those suitable dispersing or wetting agents and suspending agents mentioned herein. The sterile injectable preparation may also be a sterile injectable solution or suspension in a non-toxic parenterally acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

A compound of Formula (I), (II′), or (II), or a salt thereof may also be administered in the form of suppositories for rectal administration or sprays for nasal or inhalation use. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

### Routes of Administration

Compounds of Formula (I), (II′), or (II), or a salt thereof and compositions containing the same may be administered in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to administer the compounds of Formula (I), (II′), or (II), or a salt thereof over a defined period of time. Particular embodiments of the present invention contemplate oral administration.

### Combination Therapy

The present invention contemplates the use of compounds of Formula (I) or (II), or a salt thereof in combination with one or more active therapeutic agents (e.g., chemotherapeutic agents) or other prophylactic or therapeutic modalities (e.g., radiation). In such combination therapy, the various active agents frequently have different, complementary mechanisms of action. Such combination therapy may be especially advantageous by allowing a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, the compounds of Formula (I), (II′), or (II), or a salt thereof are administered or applied sequentially, e.g., where one agent is administered prior to one or more other agents. In other embodiments, the compounds of Formula (I) or (II), or a salt thereof are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

The compounds of Formula (I), (II′), or (II), or a salt thereof may be used in combination with at least one other (active) agent in any manner appropriate under the circumstances. In one embodiment, treatment with the at least one active agent and at least one compound of Formula (I), (II′), or (II), or a salt thereof is maintained over a period of time. In another embodiment, treatment with the at least one active agent is reduced or discontinued (e.g., when the subject is stable), while treatment with the compound of Formula (I), (II′), or (II), or a salt thereof is maintained at a constant dosing regimen. In a further embodiment, treatment with the at least one active agent is reduced or discontinued (e.g., when the subject is stable), while treatment with a compound of Formula (I), (II′), or (II), or a salt thereof is reduced (e.g., lower dose, less frequent dosing or shorter treatment regimen). In yet another embodiment, treatment with the at least one active agent is reduced or discontinued (e.g., when the subject is stable), and treatment with the compound of Formula (I), (II′), or (II), or a salt thereof is increased (e.g., higher dose, more frequent dosing or longer treatment regimen). In yet another embodiment, treatment with the at least one active agent is maintained and treatment with the compound of Formula (I), (II′), or (II), or a salt thereof is reduced or discontinued (e.g., lower dose, less frequent dosing or shorter treatment regimen). In yet another embodiment, treatment with the at least one active agent and treatment with the compound of Formula (I), (II′), or (II), or a salt thereof are reduced or discontinued (e.g., lower dose, less frequent dosing or shorter treatment regimen).

The present disclosure provides methods for treating cancer with a compound of Formula (I), (II′), or (II), or a salt thereof and at least one additional therapeutic or diagnostic agent.

In some embodiments, the compound of Formula (I), (II′), or (II), or a salt thereof is administered in combination with at least one additional therapeutic agent, selected from Temozolomide, Pemetrexed, Pegylated liposomal doxorubicin (Doxil), Eribulin (Halaven), Ixabepilone (Ixempra), Protein-bound paclitaxel (Abraxane), Oxaliplatin, Irinotecan, Venatoclax (bcl2 inhibitor), 5-azacytadine, Anti-CD20 therapeutics, such as Rituxan and obinutuzumab, Hormonal agents (anastrozole, exemestand, letrozole, zoladex, lupon eligard), CDK4/6 inhibitors, Palbociclib, Abemaciclib, CPI (Avelumab, Cemiplimab-rwlc, and Bevacizumab.

In certain embodiments, the present disclosure provides methods for treating cancer comprising administration of a compound of Formula (I), (II′), or (II), or a salt thereof described herein in combination with a signal transduction inhibitor (STI) to achieve additive or synergistic suppression of tumor growth. As used herein, the term “signal transduction inhibitor” refers to an agent that selectively inhibits one or more steps in a signaling pathway. Examples of signal transduction inhibitors (STIs) useful in methods described herein include, but are not limited to: (i) bcr/abl kinase inhibitors (e.g., GLEEVEC); (ii) epidermal growth factor (EGF) receptor inhibitors, including kinase inhibitors and antibodies; (iii) her-2/neu receptor inhibitors (e.g., HERCEPTIN); (iv) inhibitors of Akt family kinases or the Akt pathway (e.g., rapamycin); (v) cell cycle kinase inhibitors (e.g., flavopiridol); and (vi) phosphatidyl inositol kinase inhibitors. Agents involved in immunomodulation can also be used in combination with one or more compounds of Formula (I), (II′), or (II), or a salt thereof described herein for the suppression of tumor growth in cancer patients.

In certain embodiments, the present disclosure provides methods for treating cancer comprising administration of a compound of Formula (I), (II′), or (II), or a salt thereof described herein in combination with a chemotherapeutic agents. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel and doxetaxel; chlorambucil; gemcitabine; 6-thioguanine; mercaptopurine; methotrexate; platinum and platinum coordination complexes such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; PARP inhibitors such as olaparib, rucaparib, niraparib, talazoparib, veliparib, and pamiparib, DNA damage repair inhibitors such as inhibitors of ATM [such as AZ: (AZD1390) Astrazeneca's AZD0156, AZ31, AZ32; Kudos' KU-55933, KU-60019, and KU-59403; and Pfizer's CP-466722]; ATR [such as Astrazeneca's Ceralasertib (AZD6738); Repare's RP-3500; Vertex/EMD Serono's Berzosertib (VX-970/M6620); and EMD Serono's M4344; and DNA-PK (such as Astrazeneca's AZD7648; NU7441; NU7026; Kudos' KU-0060648; Vertex's VX-984; and EMD Serono's Nedisertib (M3814)] and Cyteir Therapeutics RAD51 inhibitor CYT-0851 and pharmaceutically acceptable salts, acids or derivatives of any of the above. In a particular embodiment, compounds of the present disclosure are coadministered with a cytostatic compound selected from the group consisting of cisplatin, doxorubicin, taxol, taxotere and mitomycin C. In a particular embodiment, the cytostatic compound is doxorubicin.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, enzalutamide, apalutamide, abiraterone acetate, leuprolide, and goserelin; and pharmaceutically acceptable salts, acids or derivatives of any of the above. In certain embodiments, combination therapy comprises administration of a hormone or related hormonal agent.

The present disclosure also contemplates the use of the compounds of Formula (I), (II′), or (II), or a salt thereof described herein in combination with immune checkpoint inhibitors. The tremendous number of genetic and epigenetic alterations that are characteristic of all cancers provides a diverse set of antigens that the immune system can use to distinguish tumor cells from their normal counterparts. In the case of T cells, the ultimate amplitude (e.g., levels of cytokine production or proliferation) and quality (e.g., the type of immune response generated, such as the pattern of cytokine production) of the response, which is initiated through antigen recognition by the T-cell receptor (TCR), is regulated by a balance between co-stimulatory and inhibitory signals (immune checkpoints). Under normal physiological conditions, immune checkpoints are crucial for the prevention of autoimmunity (i.e., the maintenance of self-tolerance) and also for the protection of tissues from damage when the immune system is responding to pathogenic infection. The expression of immune checkpoint proteins can be dysregulated by tumors as an important immune resistance mechanism. Examples of immune checkpoint inhibitors include but are not limited to CTLA-4, PD-1, PD-L1, BTLA, TIM3, LAG3, OX40, 41BB, VISTA, CD96, TGFβ, CD73, CD39, A2AR, A2BR, IDO1, TDO2, Arginase, B7-H3, B7-H4. Cell-based modulators of anti-cancer immunity are also contemplated. Examples of such modulators include but are not limited to chimeric antigen receptor T-cells, tumor infiltrating T-cells and dendritic-cells.

The present disclosure contemplates the use of compounds of Formula (I), (II′), or (II), or a salt thereof described herein in combination with inhibitors of the aforementioned immune-checkpoint receptors and ligands, for example ipilimumab, abatacept, nivolumab, pembrolizumab, atezolizumab, nivolumab, and durvalumab.

Additional treatment modalities that may be used in combination with a compound of Formula (I), (II′), or (II), or a salt thereof disclosed herein include radiotherapy, a monoclonal antibody against a tumor antigen, a complex of a monoclonal antibody and toxin, a T-cell adjuvant, bone marrow transplant, or antigen presenting cells (e.g., dendritic cell therapy).

The present disclosure contemplates the use of compounds of Formula (I), (II′), or (II), or a salt thereof described herein for the treatment of glioblastoma either alone or in combination with radiation and/or temozolomide (TMZ), avastin or lomustine.

The present disclosure encompasses pharmaceutically acceptable salts, acids or derivatives of any of the above.

### Dosing

The compounds of Formula (I), (II′), or (II), or a salt thereof provided herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of a compound of Formula (I), (II′), or (II), or a salt thereof, as provided herein, may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In certain embodiments, the compounds of Formula (I), (II′), or (II), or a salt thereof disclosed herein may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the compound of Formula (I), (II′), or (II), or a salt thereof is contained in a “unit dosage form”. The phrase “unit dosage form” refers to physically discrete units, each unit containing a predetermined amount of the compound of Formula (I), (II′), or (II), or a salt thereof, either alone or in combination with one or more additional agents, sufficient to produce the desired effect. It will be appreciated that the parameters of a unit dosage form will depend on the particular agent and the effect to be achieved.

### Kits

The present invention also contemplates kits comprising a compound of Formula (I), (II′), or (II), or a salt thereof, and pharmaceutical compositions thereof. The kits are generally in the form of a physical structure housing various components, as described below, and may be utilized, for example, in practicing the methods described above.

A kit can include one or more of the compound of Formula (I), (II′), or (II), or a salt thereof disclosed herein (provided in, e.g., a sterile container), which may be in the form of a pharmaceutical composition suitable for administration to a subject. The compound of Formula (I), (II′), or (II), or a salt thereof can be provided in a form that is ready for use (e.g., a tablet or capsule) or in a form requiring, for example, reconstitution or dilution (e.g., a powder) prior to administration. When the compounds of Formula (I), (II′), or (II), or a salt thereof are in a form that needs to be reconstituted or diluted by a user, the kit may also include diluents (e.g., sterile water), buffers, pharmaceutically acceptable excipients, and the like, packaged with or separately from the compounds of Formula (I), (II′), or (II), for a salt thereof. When combination therapy is contemplated, the kit may contain the several agents separately or they may already be combined in the kit. Each component of the kit may be enclosed within an individual container, and all of the various containers may be within a single package. A kit of the present invention may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### EXAMPLES

The following examples and references (intermediates) are put forth so as to provide those of ordinary skill in the art with a complete disclosure and description of how to make and use the present invention, and are not intended to limit the scope of what the inventors regard as their invention, nor are they intended to represent that the experiments below were performed or that they are all of the experiments that may be performed. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: μg=microgram; μl or μL=microliter; mM=millimolar; μM=micromolar; THF=tetrahydrofuran; DIEA=diisopropylethylamine; EtOAc=ethyl acetate; NMP=N-methylpyridine, TFA=trifluoroacetic acid; DCM=dichloromethane; Cs₂CO₃=cesium carbonate; XPhos Pd G3=2-dicyclohexylphosphino-2′,4′,6′-triisopropyl-1,1′-biphenyl)[2-(2′-amino-1,1′-biphenyl)]palladium-(II) methanesulfonate; LiCl=lithium chloride; POCl₃=phosphoryl chloride; PE=petroleum ether; DMSO=dimethylsulfoxide; HCl=hydrochloric acid; Na₂SO₄=sodium sulfate; DMF=dimethylformamide; NaOH=sodium hydroxide; K₂CO₃=potassium carbonate; MeCN=acetonitrile; BOC=tert-butoxycarbonyl; MTBE=methyl tert-butyl ether; MeOH=methanol; NaHCO₃=sodium bicarbonate; NaBH₃CN=sodium cyanoborohydride; EtOH=ethanol; PCl₅=phosphorus pentachloride; NH₄OAc=ammonium acetate; Et₂O=ether; HOAc=acetic acid; Ac₂O=acetic anhydride; i-PrOH=isopropanol; NCS=N-chlorosuccinimide; K₃PO₄=potassium phosphate; Pd(dtbpf)Cl₂=1,1′-bis(di-tert-butylphosphino)ferrocene]-dichloropalladium(II); Zn(CN)₂=Zinc cyanide; Pd(PPh₃)₄=tetrakis(triphenylphosphine)-palladium(O); Et₃N=triethylamine; CuCN=copper cyanide; t-BuONO=tert-butyl nitrite; HATU=1-[bis(dimethylamino)methylene]-1H-1,2,3-triazolo[4,5-b]pyridinium 3-oxid hexafluorophosphate; DBU=1,8-diazabicyclo(5.4.0)undec-7-ene; LiAlH₄=lithium aluminium hydride; NH₃=ammonia; H2SO₄=sulfuric acid; H₂O₂=hydrogen peroxide; NMP=N-methyl-2-pyrrolidone; MgSO4=magnesium sulphate.

### SYNTHETIC EXAMPLES

### General Procedure A

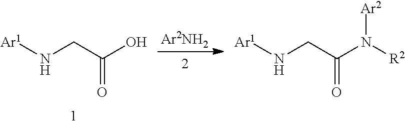

To a solution of acid 1 (1 eq.) and arylamine 2 (2 eq.) in THE (0.3M) was added DIEA (2 eq.) and propylphosphonic anhydride solution (50 wt % in EtOAc, 1.5 eq.). The mixture was stirred at room temperature overnight. The mixture was diluted with water and extracted with EtOAc. The combined organic layers were concentrated under reduced pressure.

### General Procedure B

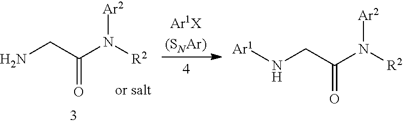

To a solution of amide 3 or it's salt (1.1 eq.) and arylhalide 4 (1 eq.) in NMP (0.5 M) was added DIEA (2.0 eq.). The mixture was stirred overnight at 50° C.

### General Procedure C

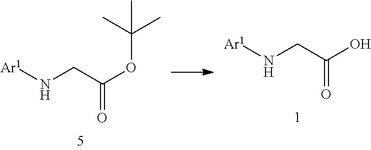

A solution of the tert-butyl ester in 25% TFA in DCM (0.2 M) was stirred for 6 h at room temperature. The mixture was concentrated under reduced pressure.

### General Procedure D

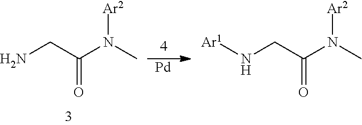

To a solution of arylhalide 4 (1 eq.) in 1,4-dioxane (0.35M) was added amide 3 (3 eq.), Cs₂CO₃ (2 eq.) and XPhos Pd G3 (57.78 mg, 0.068 mmol, 0.1 eq.). The mixture was stirred overnight at 100° C.

### General Procedure E

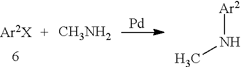

A reaction vessel under an atmosphere of nitrogen gas was charged with arylhalide 6 (1 eq.), 1,4-dioxane (0.5M), methylamine (2M in THF, 6 eq), Cs₂CO₃ (2 eq.), and t-BuXPhos-Pd-G3 (0.10 eq). The resulting mixture was heated to 100° C. and stirred overnight.

### Intermediate A

### Synthesis of 2-chloro-4,6-bis(trifluoromethyl)pyridine-3-carbonitrile

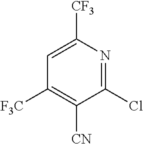 [SMILES: N#Cc1c(C(F)(F)F)cc(C(F)(F)F)nc1Cl]

To a solution of 1,1,1,5,5,5-hexafluoropentane-2,4-dione (25 g, 120 mmol) in sulfolane (50 mL) was added 2-cyanoacetamide (10 g, 120 mmol). The mixture was stirred overnight at 150° C. The mixture was diluted with EtOAc and washed with LiCl (1M). The organic layer was dried over Na₂SO₄ and concentrated under reduced pressure to give a yellow solid. The solid was dissolved in POCl₃ (36 g, 236 mmol) and Et₃N (9.6 g, 94 mmol) was added. The mixture was stirred overnight at 125° C. and then quenched with ice water. The mixture was extracted with EtOAc and the combined organic layers were washed with water and concentrated under reduced pressure. The residue was purified using silica gel chromatography (eluent: 1% EtOAc in PE) to afford (4.5 g, 35% yield) of the title compound as light-yellow oil. ¹H NMR (300 MHz; DMSO-d₆): δ 8.64 (s, 1H) ppm.

### Intermediate B

### Synthesis of (3-cyano-4,6-bis(trifluoromethyl)pyridin-2-yl)glycine

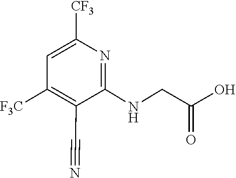 [SMILES: N#Cc1c(C(F)(F)F)cc(C(F)(F)F)nc1NCC(=O)O]

### Step 1: Preparation of tert-butyl (3-cyano-4,6-bis(trifluoromethyl)pyridin-2-yl)glycinate

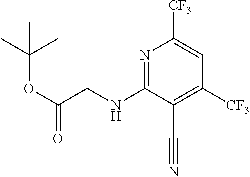 [SMILES: CC(C)(C)OC(=O)CNc1nc(C(F)(F)F)cc(C(F)(F)F)c1C#N]

To a round-bottom flask was added Intermediate A (2.00 g, 7.28 mmol), NMP (20 mL), tert-butyl glycinate (1.43 g, 10.9 mmol), and DIEA (1.88 g, 14.5 mmol). The mixture was stirred overnight at 50° C. The mixture was diluted with EtOAc (150 mL). and washed with 1M LiCl aq. (2×50 mL). The organic phase was concentrated under vacuum. The residue was purified using silica gel column (eluent: 2% EtOAc in PE) to give tert-butyl (3-cyano-4,6-bis(trifluoromethyl)pyridin-2-yl)glycinate (1.6 g, 59% yield) as a light yellow oil.

### Step 2: Preparation of (3-cyano-4,6-bis(trifluoromethyl)pyridin-2-yl)glycine

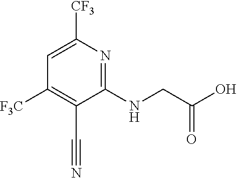 [SMILES: N#Cc1c(C(F)(F)F)cc(C(F)(F)F)nc1NCC(=O)O]

The title compound was prepared according to General procedure C using tert-butyl (3-cyano-4,6-bis(trifluoromethyl)pyridin-2-yl)glycinate (1.60 g, 4.33 mmol). The mixture was concentrated under vacuum. The residue was purified using silica gel column chromatography (eluent: 1% MeOH in DCM) to give (3-cyano-4,6-bis(trifluoromethyl)pyridin-2-yl)glycine (1.2 g, 88% yield) as a white solid.

### Example 1

### Synthesis of 2-((5-chloro-3-cyano-4,6-dimethylpyridin-2-yl)amino)-N-methyl-N-phenylacetamide

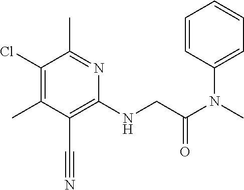

### Step 1. Preparation of (5-chloro-3-cyano-4,6-dimethylpyridin-2-yl)glycine

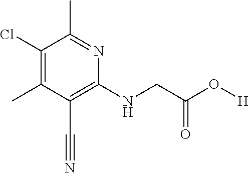 [SMILES: Cc1nc(NCC(=O)O)c(C#N)c(C)c1Cl]

To a solution of 2,5-dichloro-4,6-dimethylnicotinonitrile (5 g, 24.9 mmol) in DMSO (50 mL) was added glycine (2.1 g, 27.4 mmol) and DBU (11.4 g, 74.6 mmol). The mixture was stirred for 1 h at 150° C. and then cooled to room temperature. Water was added and the mixture was acidified to pH 3 with HCl. The mixture was extracted with EtOAc and the combined organic layers were washed with brine, dried over anhydrous Na₂SO₄ and concentrated under reduced pressure to afford the title compound (5.8 g, 97% yield) as a dark brown solid, which was used without further purification.

### Step 2. Preparation of 2-((5-chloro-3-cyano-4,6-dimethylpyridin-2-yl)amino)-N-methyl-N-phenylacetamide

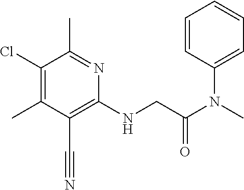

The title compound was prepared as crude using General Procedure A, using (5-chloro-3-cyano-4,6-dimethylpyridin-2-yl)glycine and N-methylaniline using the following modifications: 5-chloro-3-cyano-4,6-dimethylpyridin-2-yl)glycine was azetroped with toluene; DMF was used as the solvent. The mixture was stirred at room temperature for 3 h. The organic layer was washed with 1 M NaOH, 1 M LiCl and brine. The organic layer was dried over Na₂SO₄. The residue was preabsorbed onto silica and purified using silica gel chromatography (eluent: 0-20% (EtOAc in hexanes) to afford 2-((5-chloro-3-cyano-4,6-dimethylpyridin-2-yl)amino)-N-methyl-N-phenylacetamide. ¹H NMR (400 MHz; CDCl₃): δ 7.50-7.48 (m, 3H), 7.30-7.27 (m, 2H), 6.06 (br s, 1H), 3.96 (s, 2H), 3.33 (s, 3H), 2.47 (s, 3H), 2.43 (s, 3H) ppm. m/z 329 (M+H⁺).

### Example 2

### Synthesis of 2-((5-chloro-3-cyano-4,6-dimethylpyridin-2-yl)oxy)-N-methyl-N-phenylacetamide

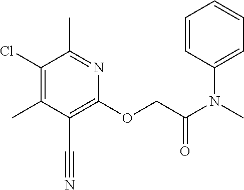

To a solution of 5-chloro-4,6-dimethyl-2-oxo-1,2-dihydropyridine-3-carbonitrile (0.032 g, 0.18 mmol) and 2-bromo-N-methyl-N-phenylacetamide (0.040 g, 0.18 mmol) in DMF (2 mL) at room temperature was added triethylamine (0.024 mL, 0.18 mmol). The mixture stirred at room temperature for 5 min. K₂CO₃ (0.048 g, 0.35 mmol) was added and the mixture stirred at room temperature overnight. The mixture was diluted with EtOAc and water and the organic layer was washed with 1 M LiCl and brine. The organic layer was dried over Na₂SO₄ and concentrated under reduced pressure. The residue was pre-absorbed onto silica and purified using silica gel chromatography (eluent: 10% EtOAc in DCM) to afford 2-((5-chloro-3-cyano-4,6-dimethylpyridin-2-yl)oxy)-N-methyl-N-phenylacetamide. ¹H NMR (400 MHz; CDCl₃): δ7.48-7.27 (m, 5H), 4.78 (s, 2H), 3.30 (s, 3H), 2.55 (s, 3H), 2.54 (s, 3H) ppm. m/z 330 (M+H⁺).

### Example 3

### Synthesis of 2-[(5-chloro-3-cyano-4,6-dimethylpyridin-2-yl)oxy]-N-(4-fluorophenyl)-N-methylacetamide

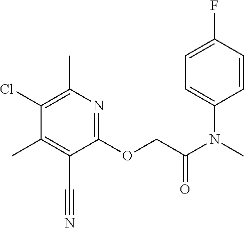

### Step 1. Preparation of tert-butyl 2-[(5-chloro-3-cyano-4,6-dimethylpyridin-2-yl)oxy]acetate

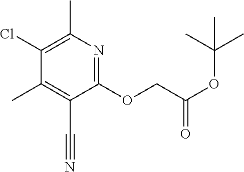 [SMILES: Cc1nc(OCC(=O)OC(C)(C)C)c(C#N)c(C)c1Cl]

To a solution of 2,5-dichloro-4,6-dimethylpyridine-3-carbonitrile (5 g, 25 mmol) in MeCN (100 mL) was added tert-butyl 2-hydroxyacetate (3.3 g, 25 mmol) and K₂CO₃ (6.9 g, 50 mmol). The mixture was heated to reflux overnight and then the mixture was filtered and the filtrate was concentrated under reduced pressure. The residue was purified using silica gel chromatography (eluent: 9% EtOAc in PE) to afford (5.8 g, 79% yield) of tert-butyl 2-[(5-chloro-3-cyano-4,6-dimethylpyridin-2-yl)oxy]acetate as a white solid.

### Step 2. Preparation of [(5-chloro-3-cyano-4,6-dimethylpyridin-2-yl)oxy]acetic acid

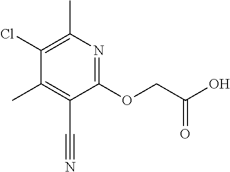 [SMILES: Cc1nc(OCC(=O)O)c(C#N)c(C)c1Cl]

The title compound was prepared using General Procedure C employing tert-butyl 2-[(5-chloro-3-cyano-4,6-dimethylpyridin-2-yl)oxy]acetate. The mixture was stirred for 6 h at room temperature and then concentrated to afford 3.6 g (87% yield) of [(5-chloro-3-cyano-4,6-dimethylpyridin-2-yl)oxy]acetic acid as a yellow solid, which was used without further purification.

### Step 3. Preparation of 2-[(5-chloro-3-cyano-4,6-dimethylpyridin-2-yl)oxy]-N-(4-fluorophenyl)-N-methylacetamide

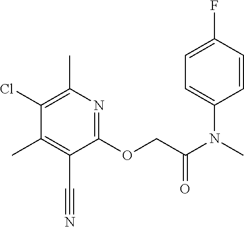

The title compound was prepared using General Procedure A employing [(5-chloro-3-cyano-4,6-dimethylpyridin-2-yl)oxy]acetic acid and 4-fluoro-N-methylaniline. The mixture was stirred overnight at room temperature and then diluted with water and extracted with EtOAc. The combined organic layers were concentrated under reduced pressure and the residue was purified by Prep-TLC (1:1, EtOAc:PE) to afford 161 mg (74% yield) of the title compound as a white solid. ¹H NMR (400 MHz; CDCl₃): δ 2.54 (s, 6H), 3.27 (s, 3H), 4.75 (s, 2H), 7.14-7.17 (m, 2H), 7.29-7.32 (m, 2H) ppm. m/z 348 (M+H⁺).

### Example 4

### Synthesis of 2-(5-chloro-3-cyano-4,6-dimethylpyridin-2-ylamino)-N-(4-fluorophenyl)-N-methylacetamide

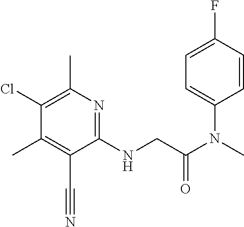 [SMILES: Cc1nc(NCC(=O)N(C)c2ccc(F)cc2)c(C#N)c(C)c1Cl]

The title compound was prepared using General Procedure A employing (5-chloro-3-cyano-4,6-dimethylpyridin-2-yl)glycine (Example 1, Step 1) and 4-fluoro-N-methylaniline. The mixture was stirred overnight at 70° C. under nitrogen and then diluted with water and extracted with EtOAc. The combined organic layers were concentrated under reduced pressure and the residue was purified by Prep-TLC with (70:1, DCM:MeOH) to afford the title compound (143 mg, 33% yield) as a white solid. ¹H NMR (300 MHz; CDCl₃): δ 2.37-2.47 (m, 6H), 3.30 (s, 3H), 3.93 (s, 2H), 7.15-7.20 (m, 2H), 7.26-7.30 (m, 2H) ppm. m/z 347 (M+H⁺).

### Example 5

### Synthesis of 2-[(5-chloro-3-cyano-4,6-dimethylpyridin-2-yl)oxy]-N-(4-chlorophenyl)-N-methylacetamide

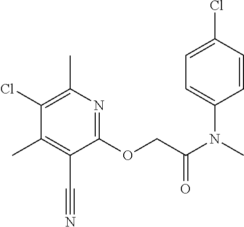 [SMILES: Cc1nc(OCC(=O)N(C)c2ccc(Cl)cc2)c(C#N)c(C)c1Cl]

The title compound was prepared using General Procedure A employing [(5-chloro-3-cyano-4,6-dimethylpyridin-2-yl)oxy]acetic acid (Example 3, Step 2) and 4-chloro-N-methylaniline. The mixture was stirred overnight at room temperature and then diluted with water and extracted with EtOA. The combined organic layers were concentrated under reduced pressure and the residue was purified using silica gel chromatography (eluent: 50% EtOAc in PE) to afford 175 mg (77% yield) of the title compound as a white solid. ¹H NMR (400 MHz; CDCl₃): δ 2.53 (s, 6H), 3.27 (s, 3H), 4.77 (s, 2H), 7.25-7.26 (m, 1H), 7.26-7.27 (m, 1H), 7.42-7.44 (m, 2H) ppm. m/z 364 (M+H⁺).

### Example 6

### Synthesis of 2-(5-chloro-3-cyano-4,6-dimethylpyridin-2-ylamino)-N-(4-chlorophenyl)-N-methylacetamide

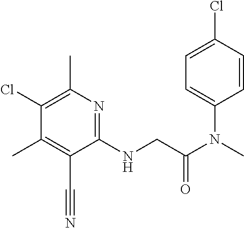 [SMILES: Cc1nc(NCC(=O)N(C)c2ccc(Cl)cc2)c(C#N)c(C)c1Cl]

The title compound was prepared using General Procedure A employing (5-chloro-3-cyano-4,6-dimethylpyridin-2-yl)glycine (Example 1, Step 1) and 4-chloro-N-methylaniline. The mixture was stirred overnight at 70° C. under nitrogen atmosphere and then diluted with water and extracted with EtOAc. The combined organic layers were concentrated under reduced pressure and the residue was purified by Prep-TLC with (70:1, DCM:MeOH) to afford the title compound (133 mg, 29% yield) as a white solid. ¹H NMR (400 MHz; CDCl₃): δ 2.52-2.44 (m, 6H), 3.30 (s, 3H), 3.91 (s, 2H), 5.91 (br s, 1H), 7.22-7.26 (m, 2H), 7.45-7.47 (m, 2H) ppm. m/z 363 (M+H⁺).

### Example 7

### Synthesis of N-(4-bromophenyl)-2-(5-chloro-3-cyano-4,6-dimethylpyridin-2-ylamino)-N-methylacetamide

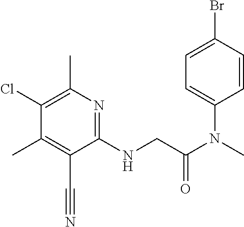 [SMILES: Cc1nc(NCC(=O)N(C)c2ccc(Br)cc2)c(C#N)c(C)c1Cl]

The title compound was prepared using General Procedure A employing (5-chloro-3-cyano-4,6-dimethylpyridin-2-yl)glycine (Example 1, Step 1) and 4-bromo-N-methylaniline. The mixture was stirred overnight at 70° C. under nitrogen and then diluted with water and extracted with EtOAc. The combined organic layers were concentrated under reduced pressure and the residue was purified by Prep-TLC with (5:1, EtOAc:hexanes) to afford the title compound (163 mg, 32% yield) as a pink solid. ¹H NMR (300 MHz; DMSO-d₆): δ 2.39 (s, 3H), 2.40 (s, 3H), 3.19 (s, 3H), 3.87 (br s, 2H), 7.05-7.09 (m, 1H), 7.38 (d, 2H), 7.68 (d, 2H) ppm. m/z 407 (M+H⁺).

### Example 8

### Synthesis of 2-(5-chloro-3-cyano-6-methylpyridin-2-ylamino)-N-methyl-N-phenylacetamide

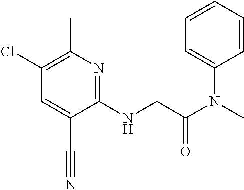 [SMILES: Cc1nc(NCC(=O)N(C)c2ccccc2)c(C#N)cc1Cl]

### Step 1. Preparation of 2,5-dichloro-6-methylpyridine-3-carbonitrile

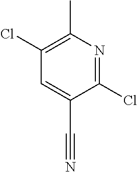

A solution of 5-chloro-6-methyl-2-oxo-1H-pyridine-3-carbonitrile (2 g, 12 mmol) in POCl₃ (5.00 mL) was heated to 120° C. for 12 h. The mixture was concentrated under reduced pressure. The residue was diluted with sat. NaHCO₃ and extracted with EtOAc. The combined organic layers were concentrated under reduced pressure and the residue was purified using silica gel chromatography (eluent: 9% EtOAc in PE) to afford (520 mg, 23% yield) of the title compound as a yellow solid.

### Step 2. Preparation of tert-butyl N-[[methyl(phenyl)carbamoyl]methyl]carbamate

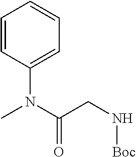 [SMILES: CN(C(=O)CNC(=O)OC(C)(C)C)c1ccccc1]

The title compound was prepared using General Procedure A employing N-methylaniline and 2-[[(tert-butoxy)carbonyl]amino]acetic acid. The mixture was stirred overnight at room temperature and then diluted with water and extracted with EtOAc. The combined organic layers were concentrated under reduced pressure and the residue was purified using silica gel chromatography (eluent: 9% EtOAc in PE) to afford the title compound (7.1 g, 72% yield) as a yellow solid.

### Step 3. Preparation of 2-amino-N-methyl-N-phenylacetamide TFA Salt

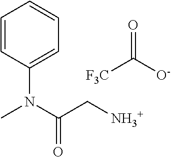

To a solution of tert-butyl N-[[methyl(phenyl)carbamoyl]methyl]carbamate (7.0 g, 26 mmol) in DCM (12 mL) was added TFA (70 mL) at room temperature. The mixture was stirred for 6 h at room temperature and then concentrated under reduced pressure. The residue was purified using silica gel chromatography (eluent: 1% MeOH in DCM) to afford the title compound (3.9 g) as a yellow solid.

### Step 4. Preparation of 2-(5-chloro-3-cyano-6-methylpyridin-2-ylamino)-N-methyl-N-phenylacetamide

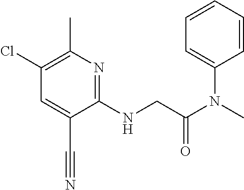 [SMILES: Cc1nc(NCC(=O)N(C)c2ccccc2)c(C#N)cc1Cl]

The title compound was prepared using General Procedure B employing 2,5-dichloro-6-methylpyridine-3-carbonitrile (Example 8, Step 1) and 2-amino-N-methyl-N-phenylacetamide TFA salt. The mixture was diluted with water and extracted with EtOAc and the combined organic layers were concentrated under reduced pressure. The residue was purified by Prep-TLC (80:1, DCM:MeOH) to afford the title compound as a white solid. ¹H NMR (400 MHz; CDCl₃): δ 2.44 (s, 3H), 3.35 (s, 3H), 3.95 (s, 2H), 6.05 (s, 1H, 7.28-7.31 (m, 2H), 7.42-7.46 (m, 1H), 7.46-7.53 (m, 2H), 7.57 (s, 1H) ppm. m/z 315 (M+H⁺).

### Example 9

### Synthesis of 2-(5-chloro-3-cyano-4-methylpyridin-2-ylamino)-N-methyl-N-phenylacetamide

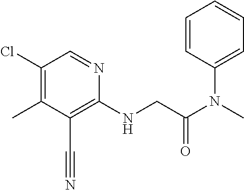 [SMILES: Cc1c(Cl)cnc(NCC(=O)N(C)c2ccccc2)c1C#N]

### Step 1. Preparation of 2,5-dichloro-4-methylnicotinonitrile

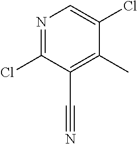

A solution of 5-chloro-2-hydroxy-4-methylpyridine-3-carbonitrile (500 mg, 3.0 mmol) in POCl₃ (3 mL) was stirred for 12 h at 120° C. Ice water was then added the mixture was extracted with EtOAc. The combined organic layers were concentrated under reduced pressure and the residue was purified using silica gel chromatography (eluent: 9% EtOAc in PE) to afford 400 mg (72% yield) of the title compound as a yellow solid.

### Step 2. Preparation of 2-(5-chloro-3-cyano-4-methylpyridin-2-ylamino)-N-methyl-N-phenylacetamide

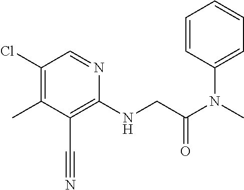 [SMILES: Cc1c(Cl)cnc(NCC(=O)N(C)c2ccccc2)c1C#N]

The title compound was prepared using General Procedure B employing 2,5-dichloro-4-methylnicotinonitrile and 2-amino-N-methyl-N-phenylacetamide TFA salt (Example 8, Step 3).

The mixture was diluted with water and extracted with EtOAc. The combined organic layers were concentrated under reduced pressure and the residue was purified using silica gel chromatography (eluent: 1% MeOH in DCM) to afford 100 mg (59% yield) of the title compound as a white solid. ¹H NMR (300 MHz; CDCl₃): δ 2.46 (s, 3H), 3.35 (s, 3H), 3.90 (s, 2H), 6.13 (s, 1H), 7.24-7.28 (m, 2H), 7.39-7.51 (m, 3H), 8.03 (s, 1H) ppm. m/z 315 (M+H⁺).

### Example 10

### Synthesis of 2-(5-chloro-4,6-dimethylpyridin-2-yloxy)-N-methyl-N-phenylacetamide

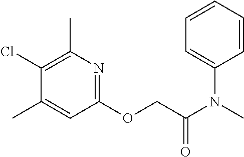 [SMILES: Cc1cc(OCC(=O)N(C)c2ccccc2)nc(C)c1Cl]

### Step 1. Preparation of 2-(benzyloxy)-N-methyl-N-phenylacetamide

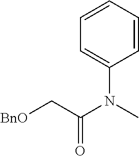 [SMILES: CN(C(=O)COCc1ccccc1)c1ccccc1]

To a solution of 2-(benzyloxy)acetic acid (4 g, 24 mmol) in DMF (40 mL) was added N-methylaniline (3.1 g, 28.9 mmol) and pyridine (5.7 g, 72.2 mmol). Then propylphosphonic anhydride solution (23 g, 36 mmol, 50 wt % in EtOAc) was added portion wise at room temperature under nitrogen and the mixture was stirred overnight at 40° C. under nitrogen. The mixture was diluted with water and extracted with EtOAc and the combined organic layers were concentrated under reduced pressure. The residue was purified using silica gel chromatography (eluent: 9% EtOAc in PE) to afford the title compound (4.2 g, 68% yield) as a white solid.

### Step 2. Preparation of 2-hydroxy-N-methyl-N-phenylacetamide

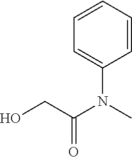

To a stirred solution of 2-(benzyloxy)-N-methyl-N-phenylacetamide (4 g, 15.7 mmol) in MeOH (50 mL) was added 10% Pd/C (800 mg) and the flask was evacuated and filled with hydrogen. The mixture was stirred overnight at room temperature under hydrogen and then filtered and the solid was washed with MeOH. The filtrate was concentrated under reduced pressure to afford the tile compound (2.5 g, 97% yield) as a brown oil, which was used without further purification.

### Step 3. Preparation of 2-(5-chloro-4,6-dimethylpyridin-2-yloxy)-N-methyl-N-phenylacetamide

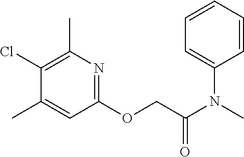 [SMILES: Cc1cc(OCC(=O)N(C)c2ccccc2)nc(C)c1Cl]

To a solution of 3,6-dichloro-2,4-dimethylpyridine (200 mg, 1.14 mmol) in 2-methoxyethyl ether (2 mL) was added 2-hydroxy-N-methyl-N-phenylacetamide (187 mg, 1.1 mmol) and K₂CO₃ (314 mg, 2.3 mmol). The mixture was stirred for 2 h at 130° C. and then diluted with water and extracted with EtOAc. The combined organic layers were concentrated under reduced pressure and the residue was purified by Prep-TLC (80:1, DCM:MeOH) to afford the title compound as a white solid. ¹H NMR (300 MHz; CDCl₃): δ 2.34 (s, 3H), 2.58 (s, 3H), 3.30 (s, 3H), 4.85 (s, 2H), 6.65 (s, 1H), 7.28-7.38 (m, 3H), 7.40-7.51 (m, 2H) ppm. m/z 305 (M+H⁺).

### Example 11

### Synthesis of N-(4-bromophenyl)-2-[(5-chloro-3-cyano-4,6-dimethylpyridin-2-yl)oxy]-N-methylacetamide

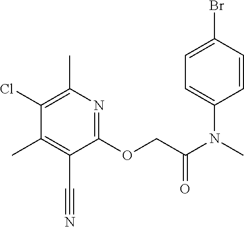 [SMILES: Cc1nc(OCC(=O)N(C)c2ccc(Br)cc2)c(C#N)c(C)c1Cl]

The title compound was prepared using General Procedure A employing [(5-chloro-3-cyano-4,6-dimethylpyridin-2-yl)oxy]acetic acid (Example 3, Step 2) and 4-bromo-N-methylaniline. The mixture was stirred overnight at room temperature and then diluted with water and extracted with EtOAc. The combined organic layers were concentrated under reduced pressure and the product was triturated with MeOH to afford 209 mg (79% yield) of the title compound as a white solid. ¹H NMR (400 MHz; CDCl₃): δ 2.53 (s, 6H), 3.27 (s, 3H), 4.78 (s, 2H), 7.20 (d, 2H), 7.59 (d, 2H) ppm. m/z 408 (M+H⁺).

### Example 12

### Synthesis of 2-[(5-chloro-4,6-dimethylpyridin-2-yl)amino]-N-methyl-N phenylacetamide

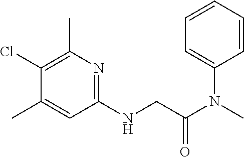 [SMILES: Cc1cc(NCC(=O)N(C)c2ccccc2)nc(C)c1Cl]

### Step 1. Preparation of 2-[(5-chloro-4,6-dimethylpyridin-2-yl)amino]acetic acid

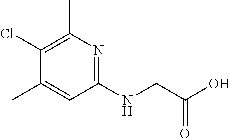

To a solution of 3,6-dichloro-2,4-dimethylpyridine (300 mg, 1.7 mmol) in DMSO (3 mL) was added 2-aminoacetic acid (192 mg, 2.6 mmol). DBU (649 mg, 4.26 mmol) was added portion wise slowly at room temperature under nitrogen and the mixture was stirred overnight at 150° C. The mixture was diluted with water and extracted with EtOAc and the combined organic layers were concentrated under reduced pressure. The residue was purified by Prep-TLC (20:1, DCM:MeOH) to afford the title compound (70 mg, 19% yield) as a white solid.

### Step 2. Preparation of 2-[(5-chloro-4,6-dimethylpyridin-2-yl)amino]-N-methyl-N-phenylacetamide

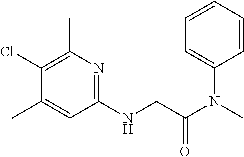 [SMILES: Cc1cc(NCC(=O)N(C)c2ccccc2)nc(C)c1Cl]

The title compound was prepared using General Procedure A employing 2-[(5-chloro-4,6-dimethylpyridin-2-yl)amino]acetic acid and N-methylaniline. The mixture was stirred overnight at 70° C. and then diluted with water and extracted with EtOAc. The combined organic layers were concentrated under reduced pressure and the residue was purified by Prep-TLC (50:1, DCM:MeOH) to afford the title compound (7 mg, 6 yield %) as a white solid. ¹H NMR (300 MHz; CDCl₃): δ 7.30-7.54 (m, 4H), 7.29-7.29 (m, 2H), 6.26 (br s, 1H), 3.80 (s, 2H), 3.29 (s, 3H), 2.49 (s, 3H), 2.24 (s, 3H) ppm. m/z 304 (M+H⁺).

### Example 13

### Synthesis of 2-(5-chloro-3-cyano-4,6-dimethylpyridin-2-yloxy)-N-(3,4-dichlorophenyl)-N-methylacetamide

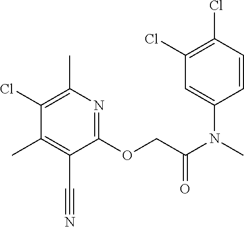 [SMILES: Cc1nc(OCC(=O)N(C)c2ccc(Cl)c(Cl)c2)c(C#N)c(C)c1Cl]

The title compound was prepared using General Procedure A employing [(5-chloro-3-cyano-4,6-dimethylpyridin-2-yl)oxy]acetic acid (Example 3, Step 2) and 3,4-dichloro-N-methylaniline. The mixture was stirred overnight at room temperature and then diluted with water and extracted with EtOAc. The combined organic layers were concentrated under reduced pressure and the residue was purified by Prep-TLC (100:1, DCM:MeOH) to afford the title compound (128 mg, 51% yield) as a white solid. ¹H NMR (300 MHz; CDCl₃): δ 7.55 (d, 1H), 7.45 (s, 1H), 7.19-7.23 (m, 1H), 4.86 (s, 2H), 3.30 (s, 3H), 2.58 (s, 6H) ppm. m/z 398 (M+H⁺).

### Example 14

### Synthesis of 2-(5-chloro-3-cyano-4,6-dimethylpyridin-2-ylamino)-N-(3,4-dichlorophenyl)-N-methylacetamide

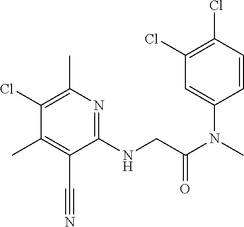 [SMILES: Cc1nc(NCC(=O)N(C)c2ccc(Cl)c(Cl)c2)c(C#N)c(C)c1Cl]

The title compound was prepared using General Procedure A employing (5-chloro-3-cyano-4,6-dimethylpyridin-2-yl)glycine (Example 1, Step 1) and 3,4-dichloro-N-methylaniline. The mixture was stirred at 70° C. overnight and then diluted with water and extracted with EtOAc. The combined organic layers were concentrated under reduced pressure and the residue was purified by Prep-TLC (30:1, DCM:MeOH) to afford the title compound as a yellow solid. ¹H NMR (400 MHz; CDCl₃): δ 2.55 (s, 6H), 3.32 (s, 3H), 3.99 (s, 2H), 5.91 (s, 1H), 7.11-7.19 (m, 1H), 7.45 (s, 1H), 7.56-7.62 (m, 1H) ppm. m/z 397 (M+H⁺).

### Example 15

### Synthesis of 2-[3-chloro-5-(trifluoromethyl)phenoxy]-N-(4-fluorophenyl)-N-methylacetamide

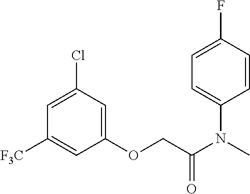 [SMILES: CN(C(=O)COc1cc(Cl)cc(C(F)(F)F)c1)c1ccc(F)cc1]

### Step 1. Preparation of tert-butyl 2-[3-chloro-5-(trifluoromethyl)phenoxy]acetate

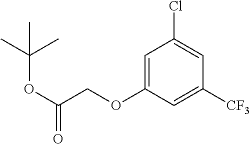 [SMILES: CC(C)(C)OC(=O)COc1cc(Cl)cc(C(F)(F)F)c1]

To a mixture of 3-chloro-5-(trifluoromethyl)phenol (500 mg, 2.5 mmol) and tert-butyl 2-bromoacetate (496 mg, 2.5 mmol) in DMF (5 mL) was added K₂CO₃ (527 mg, 3.8 mmol). The mixture was stirred for 2 h at room temperature and then diluted with water and extracted with EtOAc. The combined organic layer was concentrated under reduced pressure to afford 580 mg of the title compound as a solid. The product was used in the next step directly without further purification.

### Step 2. Preparation of 2-[3-chloro-5-(trifluoromethyl)phenoxy]acetic acid

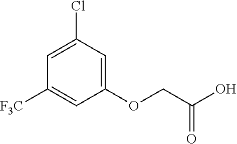 [SMILES: O=C(O)COc1cc(Cl)cc(C(F)(F)F)c1]

The title compound was prepared using General Procedure C employing tert-butyl 2-[3-chloro-5-(trifluoromethyl)phenoxy]acetate. The mixture was stirred for 8 h at room temperature. The mixture was concentrated under reduced pressure and the residue was purified using silica gel chromatography (eluent: 17% EtOAc in hexanes) to afford the title compound (150 mg, 30% yield) as a white solid.

### Step 3. Preparation of 2-[3-chloro-5-(trifluoromethyl)phenoxy]-N-(4-fluorophenyl)-N-methylacetamide

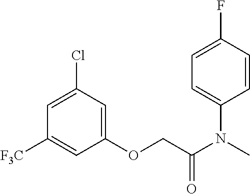 [SMILES: CN(C(=O)COc1cc(Cl)cc(C(F)(F)F)c1)c1ccc(F)cc1]

The title compound was prepared using General Procedure A employing 2-[3-chloro-5-(trifluoromethyl)phenoxy]acetic acid and 4-fluoro-N-methylaniline. The mixture was stirred overnight at 70° C. and then diluted with water and extracted with EtOAc. The residue was purified by Prep-TLC (50:1, DCM:MeOH) to the title compound (94 mg, 44% yield) as a white solid. ¹H NMR (300 MHz; CDCl₃): δ 7.25-7.34 (m, 2H), 7.17-7.25 (m, 3H), 6.93-6.96 (m, 2H), 4.45 (s, 2H), 3.30 (s, 3H) ppm. m/z 362 (M+H⁺)

### Example 16

### Synthesis of 2-[(3,5-dichlorophenyl)amino]-N-(4-fluorophenyl)-N-methylacetamide

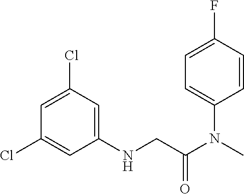 [SMILES: CN(C(=O)CNc1cc(Cl)cc(Cl)c1)c1ccc(F)cc1]

### Step 1. Preparation of tert-butyl 2-[(3,5-dichlorophenyl)amino]acetate

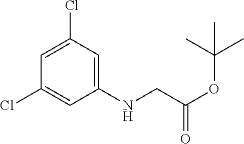 [SMILES: CC(C)(C)OC(=O)CNc1cc(Cl)cc(Cl)c1]

To a solution of 3,5-dichloroaniline (5.0 g, 30.9 mmol) in MeCN (50 mL) was added Et₃N (6.3 g, 61.7 mmol) and tert-butyl 2-bromoacetate (6.0 g, 30.9 mmol). The mixture was heated to reflux overnight and then concentrated under reduced pressure. The residue was purified using silica gel chromatography (eluent: 91% EtOAc in PE) to afford the title compound (1 g, 12% yield) as a white solid.

### Step 2. Preparation of [(3,5-dichlorophenyl)amino]acetic acid

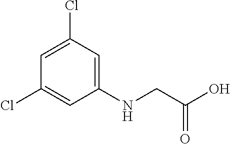 [SMILES: O=C(O)CNc1cc(Cl)cc(Cl)c1]

The title compound was prepared using General Procedure C employing tert-butyl 2-[(3,5-dichlorophenyl)amino]acetate. The residue was used without further purification.

### Step 3. Preparation of 2-[(3,5-dichlorophenyl)amino]-N-(4-fluorophenyl)-N-methylacetamide

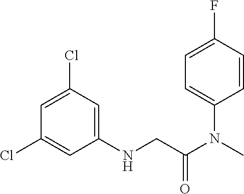 [SMILES: CN(C(=O)CNc1cc(Cl)cc(Cl)c1)c1ccc(F)cc1]

The title compound was prepared using General Procedure A employing 2-[(3,5-dichlorophenyl)amino]acetic acid and 4-fluoro-N-methylaniline. The residue was purified by Prep-TLC with (80:1, DCM:MeOH) to afford the title compound (154 mg, 69% yield) as a white solid. ¹H NMR (400 MHz; CDCl₃): δ 3.33 (s, 3H), 3.50 (s, 2H), 6.32 (s, 2H), 6.67 (s, 1H), 7.19-7.31 (m, 5H) ppm. m/z 327 (M+H⁺).

### Example 17

### Synthesis of 2-[(5-chloro-3-cyano-4,6-dimethylpyridin-2-yl)amino]-N-(4-chlorophenyl)-N-(propan-2-yl)acetamide

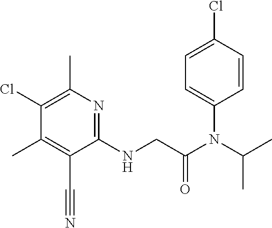 [SMILES: Cc1nc(NCC(=O)N(c2ccc(Cl)cc2)C(C)C)c(C#N)c(C)c1Cl]

### Step 1. Preparation of 4-chloro-N-isopropylaniline

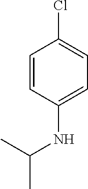

To a solution of 4-chloroaniline (3 g, 23.5 mmol) in EtOH (150 mL), HOAc (1.5 mL) and acetone (3.4 mL, 47 mmol) under nitrogen was added MgSO₄ (12 g, 100 mmol) and NaBH₃CN (2.96 g, 47 mmol). The mixture was stirred overnight at room temperature and then diluted with water and extracted with EtOAc. The combined organic layers were concentrated under reduced pressure and the residue was purified using silica gel chromatography (eluent: 0-100% EtOAc in PE) to afford 4-chloro-N-(propan-2-yl)aniline (3.5 g, 88% yield) as a yellow oil.

### Step 2. Preparation of 2-chloro-N-(4-chlorophenyl)-N-isopropylacetamide

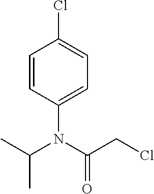

To a solution of 4-chloro-N-isopropylaniline (1.0 g, 5.9 mmo) and chloroacetyl chloride (0.67 g, 5.9 mmol in DCM (10 mL) was added Et₃N (1.2 g, 11.8 mmo). The mixture was stirred at room temperature under nitrogen for 2 h and then washed with water. The organic layer was concentrated under reduced pressure and the residue was purified using silica gel chromatography (eluent: 2% EtOAc in PE) to afford the title compound (1.02 g, 70% yield) as a yellow solid.

### Step 3. Preparation of 2-amino-N-(4-chlorophenyl)-N-isopropylacetamide

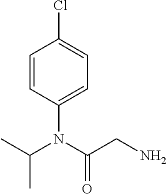

2-Chloro-N-(4-chlorophenyl)-N-isopropylacetamide (1.0 g, 4.1 mmol) was diluted with NH₃ in MeOH (250 mL, 8 M) and the mixture was stirred overnight at 50° C. The mixture was concentrated under reduced pressure and the residue was purified by Prep-TLC (50:1, DCM:MeOH) to afford the title compound (800 mg, 87% yield) as a white solid.

### Step 4. Preparation of 2-[(5-chloro-3-cyano-4,6-dimethylpyridin-2-yl)amino]-N-(4-chloro-phenyl)-N-(propan-2-yl)acetamide

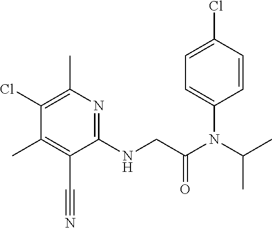 [SMILES: Cc1nc(NCC(=O)N(c2ccc(Cl)cc2)C(C)C)c(C#N)c(C)c1Cl]

The title compound was prepared using General Procedure B employing 2-amino-N-(4-chlorophenyl)-N-isopropylacetamide and 2,5-dichloro-4,5-nicotinonitrile. The residue was purified by Prep-TLC (100:1, DCM:MeOH) to afford the title compound (79 mg, 27% yield) as a white solid. ¹H NMR (300 MHz, DMSO-d₆): δ 1.00 (d, 6H), 2.40 (s, 3H), 2.41 (s, 3H), 3.60 (d, 2H), 4.77-4.81 (m, 1H), 7.03-7.06 (m, 1H), 7.35-7.38 (m, 2H), 7.58-7.61 (m, 2H) ppm. m/z 391 (M+H⁺).

### Example 18

### Synthesis of 2-[[3-chloro-5-(trifluoromethyl)phenyl]amino]-N-(4-fluorophenyl)-N-methylacetamide

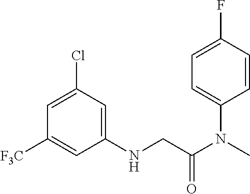 [SMILES: CN(C(=O)CNc1cc(Cl)cc(C(F)(F)F)c1)c1ccc(F)cc1]

### Step 1. Preparation of tert-butyl 2-[[3-chloro-5-(trifluoromethyl)phenyl]amino]acetate

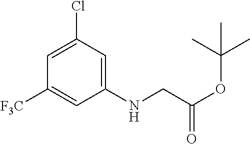 [SMILES: CC(C)(C)OC(=O)CNc1cc(Cl)cc(C(F)(F)F)c1]

To a solution of 3-chloro-5-(trifluoromethyl)aniline (700 mg, 3.6 mmol) in MeCN (7 mL) was added tert-butyl 2-bromoacetate (698 mg, 3.6 mmol) and K₂CO₃ (989 mg, 7.2 mmol). The mixture was stirred overnight at 80° C. and then diluted with water and extracted with EtOAc. The residue was purified by Prep-TLC (50:1, DCM:MeOH) to afford the title compound (400 mg, 36% yield) as a white solid.

### Step 2. Preparation of 2-[[3-chloro-5-(trifluoromethyl)phenyl]amino]acetic acid

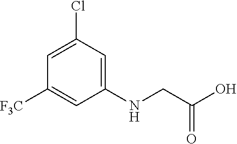 [SMILES: O=C(O)CNc1cc(Cl)cc(C(F)(F)F)c1]

The title compound was prepared using General Procedure C employing tert-butyl 2-[[3-chloro-5-(trifluoromethyl)phenyl]amino]acetate. The mixture was stirred for 3 h and then quenched with sat. NaHCO₃ and extracted with EtOAc. The combined organic layers were concentrated under reduced pressure and the residue was purified by Prep-TLC (20:1, DCM:MeOH) to afford the title compound (200 mg, 61% yield) as a white solid.

### Step 3. Preparation of 2-[[3-chloro-5-(trifluoromethyl)phenyl]amino]-N-(4-fluorophenyl)-N-methylacetamide

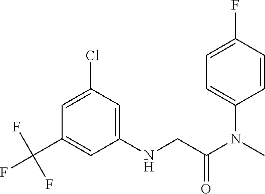 [SMILES: CN(C(=O)CNc1cc(Cl)cc(C(F)(F)F)c1)c1ccc(F)cc1]

The title compound was prepared using General Procedure A employing 2-(3-chloro-5-(trifluoromethyl)phenylamino)acetic acid and 4-fluoro-N-methylaniline. The mixture was stirred overnight at 70° C. and then diluted with water and extracted with EtOAc. The mixture was concentrated under reduced pressure and the residue was purified by Prep-TLC (100:1, DCM:MeOH) to afford the title compound (16 mg, 6% yield) as a white solid. ¹H NMR (300 MHz; CDCl₃): δ 3.34 (s, 3H), 3.53 (d, 2H), 6.56 (d, 2H), 6.92 (s, 1H), 7.20-7.30 (m, 5H) ppm. m z 361 (M+H⁺).

### Example 19

### Synthesis of 2-[(3-cyano-4-methylquinolin-2-yl)amino]-N-(4-fluorophenyl)-N-methylacetamide

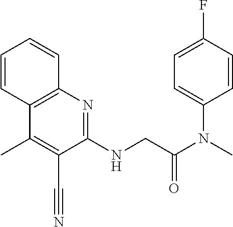 [SMILES: Cc1c(C#N)c(NCC(=O)N(C)c2ccc(F)cc2)nc2ccccc12]

### Step 1. Preparation of tert-butyl 2-((4-fluorophenyl)(methyl)amino)-2-oxoethylcarbamate

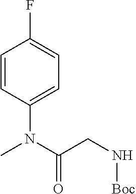 [SMILES: CN(C(=O)CNC(=O)OC(C)(C)C)c1ccc(F)cc1]

To a solution of 2-[[(tert-butoxy)carbonyl]amino]acetic acid (14 g, 80 mmol) and 4-fluoro-N-methylaniline (10 g, 80 mmol) in THF (100 mL) was added DIEA (20.6 g, 160 mmol) and propylphosphonic anhydride solution (76 g, 120 mmol, 50 wt % in EtOAc) at room temperature under nitrogen atmosphere. The mixture was stirred overnight at room temperature under nitrogen atmosphere and then diluted with water and extracted with EtOAc. The combined organic layers were concentrated under reduced pressure and the residue was purified using silica gel chromatography (eluent: 5% EtOAc in PE) to afford the title compound (22 g, 98% yield) as a white solid.

### Step 2. Preparation of 2-amino-N-(4-fluorophenyl)-N-methylacetamide TFA salt

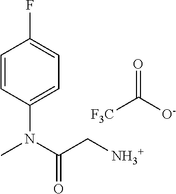

To a solution of tert-butyl N-[[(4-fluorophenyl)(methyl)carbamoyl]methyl]carbamate (22 g, 78 mmol) in DCM (190 mL) was added TFA (60 mL) at room temperature under nitrogen atmosphere. The mixture was stirred overnight at room temperature under nitrogen atmosphere and then concentrated under reduced pressure. A solution of sat. Na₂CO₃ was added at room temperature and the aqueous layer was extracted with DCM. The mixture was concentrated under reduced pressure and the residue was purified using silica gel chromatography (eluent: 2% MeOH in DCM) to afford the title compound (7.5 g, 53% yield).

### Step 3. Preparation of 2-[(3-cyano-4-methylquinolin-2-yl)amino]-N-(4-fluorophenyl)-N-methylacetamide

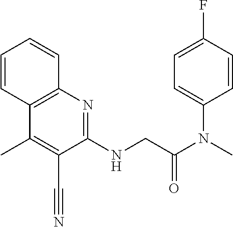 [SMILES: Cc1c(C#N)c(NCC(=O)N(C)c2ccc(F)cc2)nc2ccccc12]

The title compound was prepared using General Procedure B employing 2-amino-N-(4-fluorophenyl)-N-methylacetamide TFA salt and 2-chloro-4-methylquinoline-3-carbonitrile. The mixture was stirred at 140° C. overnight and then diluted with water and extracted with EtOAc. The residue was purified by Prep-TLC with (10:1, DCM:MeOH) to afford the title compound (75 mg, 26% yield) as a yellow solid. ¹H NMR (300 MHz; DMSO-d₆): δ 7.95 (d, 1H), 7.64-7.69 (m, 1H), 7.53-7.56 (m, 3H), 7.30-7.38 (m, 3H), 6.96-7.00 (m, 1H), 3.91 (s, 2H), 3.21 (s, 3H), 2.81 (s, 3H) ppm. m/z 349 (M+H⁺).

### Example 20

### Synthesis of 2-(4-cyano-1-methyl-6,7-dihydro-5H-cyclopenta[c]pyridin-3-ylamino)-N-(4-fluorophenyl)-N-methylacetamide

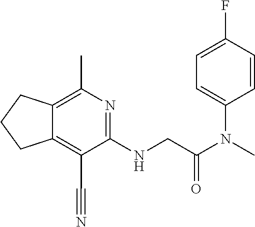 [SMILES: Cc1nc(NCC(=O)N(C)c2ccc(F)cc2)c(C#N)c2c1CCC2]

### Step 1. Preparation of 3-hydroxy-1-methyl-6,7-dihydro-5H-cyclopenta[c]pyridine-4-carbonitrile

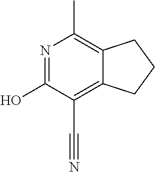

To a solution of 2-acetylcyclopentan-1-one (3 g, 24 mmol) in EtOH (30 mL) was added 2-cyanoacetamide (2.0 g, 24 mmol) and piperidine (4.05 g, 448 mmol). The mixture was stirred overnight at 80° C. under nitrogen atmosphere and then concentrated under reduced pressure. The crude product was purified by re-crystallization from MeOH to give 3.4 g (82% yield) of the title compound as a white solid.

### Step 2. Preparation of 3-chloro-1-methyl-6,7-dihydro-5H-cyclopenta[c]pyridine-4-carbonitrile

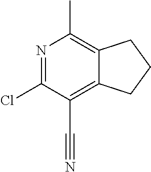

A solution of 3-hydroxy-1-methyl-6,7-dihydro-5H-cyclopenta[c]pyridine-4-carbonitrile (3.4 g, 20 mmol), PCl₅ (20 g, 98 mmol) and POCl₃ (17 mL) was heated to 120° C. overnight. The mixture was concentrated under reduced pressure, diluted with EtOAc and washed with sat. NaHCO₃. The organic layer was concentrated under reduced pressure and the crude product was purified by re-crystallization from PE to afford 1.1 g (29% yield) of the title compound as a white solid.

### Step 3. Preparation of 2-(4-cyano-1-methyl-6,7-dihydro-5H-cyclopenta[c]pyridin-3-ylamino)-N-(4-fluorophenyl)-N-methylacetamide

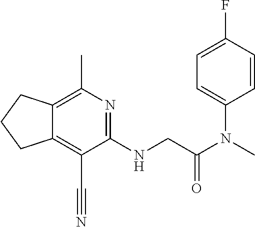 [SMILES: Cc1nc(NCC(=O)N(C)c2ccc(F)cc2)c(C#N)c2c1CCC2]

The title compound was prepared using General Procedure B employing 3-chloro-1-methyl-6,7-dihydro-5H-cyclopenta[c]pyridine-4-carbonitrile and 2-amino-N-(4-fluorophenyl)-N-methylacetamide TFA salt (Example 19, Step 2). The mixture was stirred overnight at 100° C. and then diluted with water and extracted with EtOAc. The residue was purified using silica gel chromatography (eluent: 5% MeOH in DCM) to give a residue. The residue was further purified by Prep-HPLC (Sunfire Prep C₁₈ OBD column; gradient elution 60 to 78% MeCN in water, with both eluents containing 0.05% TFA) to give a residue. The residue was even further purified by re-crystallization from MeOH to afford 29 mg (6% yield) of the title compound as a red solid. ¹H NMR (300 MHz): δ 7.41-7.52 (m, 2H), 7.29-7.35 (m, 2H), 6.67 (t, 1H), 3.78 (br s, 2H), 3.16 (s, 3H), 2.87 (t, 2H), 2.70 (t, 2H), 2.23 (s, 3H), 1.95-2.05 (m, 2H) ppm. m/z 339 (M+H⁺).

### Example 21

### Synthesis of 2-([3-cyano-4-methyl-5H,6H,7H-cyclopenta[b]pyridin-2-yl]amino)-N-(4-fluorophenyl)-N-methylacetamide

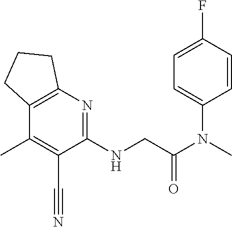 [SMILES: Cc1c(C#N)c(NCC(=O)N(C)c2ccc(F)cc2)nc2c1CCC2]

### Step 1. Preparation of 2-hydroxy-4-methyl-6,7-dihydro-5H-cyclopenta[b]pyridine-3-carbonitrile

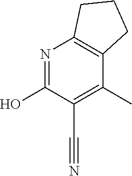

To a solution of ethyl 2-cyanoacetate (5 g, 44 mmol) in EtOH (50 mL) was added cyclopentanone (1.9 g, 22 mmol), NH₄OAc (6.8 g, 88 mmol), and acetaldehyde (2.0 g, 44 mmol). The mixture was stirred at 80° C. overnight and then diluted with water. The solids were collected by filtration and the crude product was purified by re-crystallization from MeOH to give 800 mg (10% yield) of the title compound as a white solid.

### Step 2. Preparation of 2-chloro-4-methyl-6,7-dihydro-5H-cyclopenta[b]pyridine-3-carbonitrile

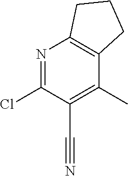

2-Hydroxy-4-methyl-6,7-dihydro-5H-cyclopenta[b]pyridine-3-carbonitrile (800 mg, 4.6 mmol) was diluted with POCl₃ (4 mL) and the mixture was stirred at 120° C. overnight. The mixture was concentrated under reduced pressure and then diluted with water and extracted with EtOAc. The combined organic layers were concentrated under reduced pressure and the crude product was purified by re-crystallization from MeOH to give 500 mg (57% yield) of the title compound as a grey solid.

### Step 3. Preparation of 2-([3-cyano-4-methyl-5H,6H,7H-cyclopenta[b]pyridin-2-yl]amino)-N-(4-fluorophenyl)-N-methylacetamide

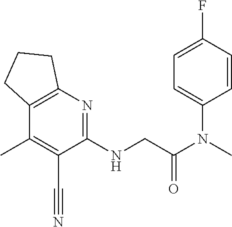 [SMILES: Cc1c(C#N)c(NCC(=O)N(C)c2ccc(F)cc2)nc2c1CCC2]

The title compound was prepared using General Procedure B employing 2-chloro-4-methyl-5H,6H,7H-cyclopenta[b]pyridine-3-carbonitrile and 2-amino-N-(4-fluorophenyl)-N-methylacetamide TFA salt (Example 19, Step 2). The mixture was stirred at 100° C. overnight and then diluted with water and extracted with EtOAc. The combined organic layers were concentrated under reduced pressure and the residue was purified by Prep-TLC (1:1, EtOAc:PE) to give a residue. The residue was re-crystallization from MeOH to afford 51 mg (15% yield) of the title compound as a white solid. ¹H NMR (400 MHz; DMSO-d₆): δ 7.45-7.58 (m, 2H), 7.30-7.35 (m, 2H), 6.60 (t, 1H), 3.77 (s, 2H), 3.17 (s, 3H), 2.69-2.79 (m, 4H), 2.26 (s, 3H), 1.94-2.01 (m, 2H) ppm. m/z 339 (M+H⁺)

### Example 22

### Synthesis of 2-((5-chloro-3-cyano-4,6-dimethylpyridin-2-yl)amino)-N-(2,4-difluorophenyl)-N-methylacetamide

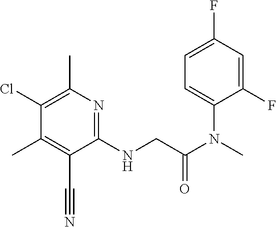 [SMILES: Cc1nc(NCC(=O)N(C)c2ccc(F)cc2F)c(C#N)c(C)c1Cl]

The title compound was prepared using General Procedure A employing (5-chloro-3-cyano-4,6-dimethylpyridin-2-yl)glycine (Example 1, Step 1) and 2,4-difluoro-N-methylaniline. The mixture was stirred overnight at 70° C. and then diluted with water and extracted with EtOAc. The combined organic layers were concentrated under reduced pressure and the residue was purified using silica gel chromatography (eluent: 9% EtOAc in PE) to give a residue. The residue was further purified by reverse flash chromatography (C₁₈ silica gel column; eluent: 10% to 50% MeOH in water) to afford the title compound as a solid. ¹H NMR (300 MHz; CDCl₃): δ 7.37-7.28 (m, 1H), 7.04-7.01 (m, 2H), 5.94 (br s, 1H), 4.05-3.98 (m, 1H), 3.86-3.80 (m, 1H), 3.27 (s, 3H), 2.46 (s, 3H), 2.44 (s, 3H) ppm. m/z 365 (M+H⁺).

### Example 23

### Synthesis of (S)-2-((5-chloro-3-cyano-4,6-dimethylpyridin-2-yl)amino)-N-(4-fluorophenyl)-N-methylpropanamide

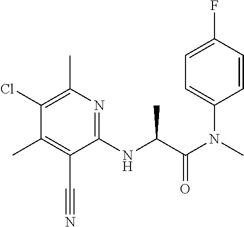 [SMILES: Cc1nc(N[C@@H](C)C(=O)N(C)c2ccc(F)cc2)c(C#N)c(C)c1Cl]

### Step 1. Preparation of tert-butyl (S)-(1-((4-fluorophenyl)(methyl)amino)-1-oxopropan-2-yl)carbamate

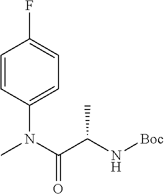 [SMILES: C[C@H](NC(=O)OC(C)(C)C)C(=O)N(C)c1ccc(F)cc1]

The title compound was prepared using General Procedure A employing 4-fluoro-N-methylaniline and Boc-Ala-OH. The mixture was diluted with water and EtOAc. The organic layer was washed with 1 M NaOH, 1 M HCl and then brine. The organic layer was dried over Na₂SO₄ and concentrated under reduced pressure to afford the title compound which was used without further purification.

### Step 2. Preparation of (S)-2-amino-N-(4-fluorophenyl)-N-methylpropanamide hydrochloride

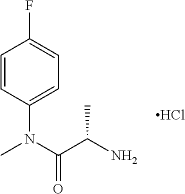

To a solution of tert-butyl (S)-(1-((4-fluorophenyl)(methyl)amino)-1-oxopropan-2-yl)carbamate (0.33 mg, 1.1 mmol) in THE (6 mL) was added HCl (4 N in 1,4-dioxane, 2.7 mL, 11 mmol). The mixture was stirred at room temperature overnight and then concentrated under reduced pressure. The residue was triturated with Et₂O to give the title compound as a solid.

### Step 3. Preparation of(S)-2-((5-chloro-3-cyano-4,6-dimethylpyridin-2-yl)amino)-N-(4-fluorophenyl)-N-methylpropanamide

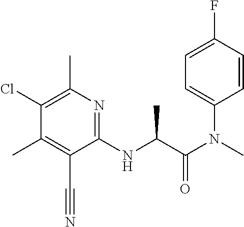 [SMILES: Cc1nc(N[C@@H](C)C(=O)N(C)c2ccc(F)cc2)c(C#N)c(C)c1Cl]

The title compound was prepared using General Procedure B employing (S)-2-amino-N-(4-fluorophenyl)-N-methylpropanamide hydrochloride, 2,5-dichloro-4,6-dimethylpyridine-3-carbonitrile and DIEA (4.0 eq.). The mixture was heated to 100° C. overnight. The mixture was diluted with EtOAc and washed with water and then brine. The organic layer was dried over Na₂SO₄. The residue was preabsorbed onto silica and purified using silica gel chromatography (eluent: 0-50% EtOAc in hexanes) to afford the title compound. ¹H NMR (400 MHz; CDCl₃): δ 1.26 (d, 3H), 2.47 (s, 3H), 2.51 (s, 3H), 3.27 (s, 3H), 4.82-4.85 (m, 1H), 5.71 (br s, 1H), 7.13-7.19 (m, 2H), 7.36-7.39 (m, 2H) ppm. m/z 361 (M+H⁺).

### Example 24

### Synthesis of 2-(4-cyano-6,7-dihydro-5H-cyclopenta[c]pyridin-3-ylamino)-N-(4-fluorophenyl)-N-methylacetamide

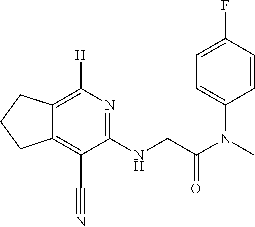 [SMILES: CN(C(=O)CNc1ncc2c(c1C#N)CCC2)c1ccc(F)cc1]

### Step 1. Preparation of 2-cyclopentylidenepropanedinitrile

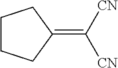

To a solution of cyclopentanone (8.4 g, 99.9 mmol) in toluene (84 mL) was added malononitrile (6.6 g, 99.9 mmol), HOAc (4.8 g, 79.9 mmol), NH₄OAc (1.5 g, 20 mmol) under nitrogen. The mixture was stirred overnight at 120° C. and then diluted with water and extracted with EtOAc. The residue was purified using silica gel chromatography (eluent: 9% EtOAc in PE) to afford 2-cyclopentylidenepropanedinitrile (7 g, 50% yield) as a yellow oil.

### Step 2. Preparation of 3-chloro-6,7-dihydro-5H-cyclopenta[c]pyridine-4-carbonitrile

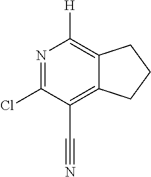 [SMILES: N#Cc1c(Cl)ncc2c1CCC2]

To a solution of 2-cyclopentylidenepropanedinitrile (3.0 g, 23 mmol) in toluene (30 mL) was added Ac₂O (2.8 g, 4.5 mmol) and N,N-dimethylformamide dimethyl acetal (3.2 g, 27.3 mmol) under nitrogen. The mixture was stirred overnight at room temperature under nitrogen and then concentrated under reduced pressure. The residue was dissolved with i-PrOH (30 mL) and HCl (4M in 1,4-dioxane, 34 mL, 136 mmol) was added. The mixture was stirred for 4 h at room temperature and then diluted with water and extracted with EtOAc. The residue was purified using silica gel chromatography (eluent: 9% EtOAc in PE) to the title compound (1.1 g, 26% yield) as a yellow solid.

### Step 3. Preparation of 2-(4-cyano-6,7-dihydro-5H-cyclopenta[c]pyridin-3-ylamino)-N-(4-fluorophenyl)-N-methylacetamide

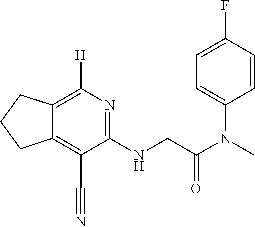 [SMILES: CN(C(=O)CNc1ncc2c(c1C#N)CCC2)c1ccc(F)cc1]

The title compound was prepared using General Procedure B employing 3-chloro-6,7-dihydro-5H-cyclopenta[c]pyridine-4-carbonitrile and 2-amino-N-(4-fluorophenyl)-N-methylacetamide TFA salt (Example 19, Step 2). The mixture was stirred overnight at 100° C. and then diluted with water and extracted with EtOAc. The organic layer was concentrated under reduced pressure and the residue was purified using silica gel chromatography (eluent: 2% MeOH in DCM) to afford the title compound as a white solid. ¹H NMR (400 MHz; DMSO-d₆): δ 8.07 (s, 1H), 7.45-7.53 (m, 2H), 7.30-7.38 (m, 2H), 6.65 (s, 1H), 3.72-3.84 (m, 2H), 3.17 (s, 3H), 2.90 (t, 2H), 2.76 (t, 2H), 2.01-2.05 (m, 2H) ppm. m/z 325 (M+H⁺).

### Example 25

### Synthesis of 2-(3,5-dichloro-4,6-dimethylpyridin-2-ylamino)-N-(4-fluorophenyl)-N-methylacetamide

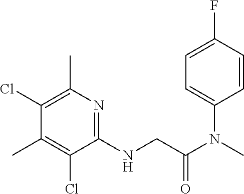 [SMILES: Cc1nc(NCC(=O)N(C)c2ccc(F)cc2)c(Cl)c(C)c1Cl]

### Step 1. Preparation of 3,5-dichloro-4,6-dimethylpyridin-2-amine

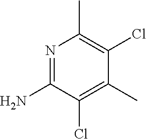

To a solution of 4,6-dimethylpyridin-2-amine (2.7 g, 22 mmol) in DMF (27 mL) was added NCS (5.0 g, 37.6 mmol). The mixture was stirred at 50° C. under nitrogen atmosphere for 4 h and then diluted with water and extracted with DCM. The mixture was concentrated under reduced pressure and the residue was purified using silica gel chromatography (eluent: 3% EtOAc in PE) to afford the title compound (1.3 g, 81% yield) as a yellow oil.

### Step 2. Preparation of tert-butyl 2-[(3,5-dichloro-4,6-dimethylpyridin-2-yl)amino]acetate

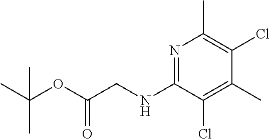 [SMILES: Cc1nc(NCC(=O)OC(C)(C)C)c(Cl)c(C)c1Cl]

To a solution of 3,5-dichloro-4,6-dimethylpyridin-2-amine (1.1 g, 6 mmol) in DMF (11 mL) was added tert-butyl 2-bromoacetate (1.12 g, 6 mmol), and K₂CO₃ (1.59 g, 12 mmol). The mixture was stirred at 90° C. overnight and then diluted with water and extracted with EtOAc. The combined organic layers were concentrated under reduced pressure. The residue was purified using silica gel chromatography (eluent: 99% EtOAc in PE) to afford the title compound (156 mg, 9% yield) as a yellow-green solid.

### Step 3. Preparation of 2-[(3,5-dichloro-4,6-dimethylpyridin-2-yl)amino]acetic acid

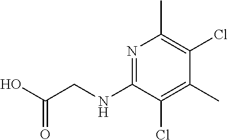

The title compound was prepared using General Procedure C employing tert-butyl 2-[(3,5-dichloro-4,6-dimethylpyridin-2-yl)amino]acetate. The mixture was stirred at room temperature overnight and then concentrated under reduced pressure. The residue was purified by Prep-TLC (80:1, DCM:MeOH) to afford the title compound (70 mg, 86% yield) as a yellow solid

### Step 4. Preparation of 2-(3,5-dichloro-4,6-dimethylpyridin-2-ylamino)-N-(4-fluorophenyl)-N-methylacetamide

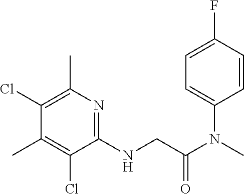 [SMILES: Cc1nc(NCC(=O)N(C)c2ccc(F)cc2)c(Cl)c(C)c1Cl]

The title compound was prepared using General Procedure A employing 2-[(3,5-dichloro-4,6-dimethylpyridin-2-yl)amino]acetic acid and 4-fluoro-N-methylaniline. The mixture was stirred overnight at 70° C. and then diluted with water and extracted with EtOAc. The combined organic layer was concentrated under reduced pressure and the residue was purified by Prep-TLC (200:1, DCM:EtOAc) to afford the title compound (2.7 mg, 3% yield) as a white solid. ¹H NMR (300 MHz; CDCl₃): δ 2.40 (s, 3H), 2.42 (s, 3H), 3.32 (s, 3H), 3.90 (d, 2H), 5.8 (br s, 1H), 7.17-7.29 (m, 2H), 7.40-7.44 (m, 2H) ppm. m/z 356 (M+H⁺).

### Example 26

### Synthesis of 2-((3,5-bis(trifluoromethyl)phenyl)amino)-N-(4-fluorophenyl)-N-methylacetamide

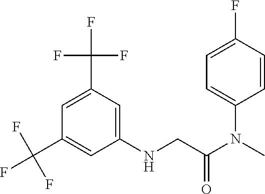 [SMILES: CN(C(=O)CNc1cc(C(F)(F)F)cc(C(F)(F)F)c1)c1ccc(F)cc1]

The title compound was prepared using General Procedure B employing 2-amino-N-(4-fluorophenyl)-N-methylacetamide TFA salt (Example 19, Step 2) and 1-bromo-3,5-bis(trifluoromethyl)benzene and then diluted with water and extracted with EtOAc. The mixture was concentrated under reduced pressure and the residue was purified by Prep-TLC (200:1, DCM:MeOH) to afford the title compound (141 mg, 52% yield) as a white solid. ¹H NMR (300 MHz; CDCl₃): δ 3.35 (s, 3H), 3.59 (s, 2H), 6.79 (s, 2H), 7.21-7.24 (m, 1H), 7.24-7.31 (m, 4H) ppm. m/z 395 (M+H⁺).

### Example 27

### Synthesis of 2-(5-chloro-3-cyano-4,6-dimethylpyridin-2-ylamino)-N-(4-methoxyphenyl)-N-methylacetamide

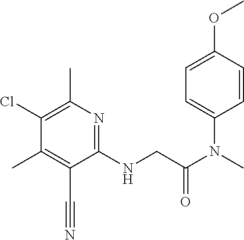

The title compound was prepared using General Procedure A employing (5-chloro-3-cyano-4,6-dimethylpyridin-2-yl)glycine and 4-methoxy-N-methylaniline (3 eq.). The mixture was diluted with water and extracted with EtOAc. The combined organic layers were concentrated under reduced pressure and the residue was purified by Prep-TLC (40:1, DCM:MeOH) to afford 53 mg of the title compound as an off-white solid. ¹H NMR (400 MHz; CDCl₃): δ 7.17-7.20 (m, 2H), 6.97-7.00 (m, 2H), 6.03 (brs, 1H), 3.93-3.86 (m, 2H), 3.83 (s, 3H), 3.30 (s, 3H), 2.52-2.44 (m, 6H) ppm. m/z 359 (M+H⁺).

### Example 28

### Synthesis of 2-((5-chloro-3-cyano-4,6-dimethylpyridin-2-yl)amino)-N-(3-cyanophenyl)-N-methylacetamide

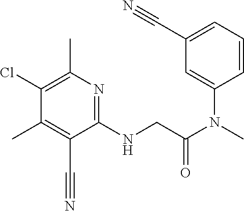 [SMILES: Cc1nc(NCC(=O)N(C)c2cccc(C#N)c2)c(C#N)c(C)c1Cl]

The title compound was prepared using General Procedure A employing (5-chloro-3-cyano-4,6-dimethylpyridin-2-yl)glycine (Example 1, Step 1) and 3-(methylamino)benzonitrile (1 eq.). The mixture was stirred overnight at 70° C. under nitrogen atmosphere and then diluted with water and extracted with EtOAc. The mixture was concentrated under reduced pressure and the residue was purified by Prep-TLC (100:1, DCM:MeOH) to afford the title compound (47 mg, 16% yield) as a pink solid. ¹H NMR (400 MHz; DMSO-d₆): δ 2.39 (s, 3H), 2.42 (s, 3H), 3.24 (s, 3H), 3.90-3.95 (m, 2H), 7.14-7.17 (m, 1H), 7.66-7.70 (m, 1H), 7.78-7.78 (m, 1H), 7.83-7.84 (m, 1H), 7.97 (s, 1H) ppm. m/z 354 (M+H⁺).

### Example 29

### Synthesis of 2-[(3-bromo-5-chlorophenyl)amino]-N-(4-fluorophenyl)-N-methylacetamide

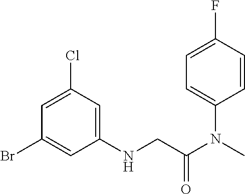 [SMILES: CN(C(=O)CNc1cc(Cl)cc(Br)c1)c1ccc(F)cc1]

The title compound was prepared using General Procedure D employing 2-amino-N-(4-fluorophenyl)-N-methylacetamide TFA salt (Example 19, Step 2) and 1,3-dibromo-5-chlorobenzene. The mixture was quenched with water and extracted with EtOAc. The combined organic layers were concentrated under reduced pressure and the residue was purified by Prep-TLC (50:1 DCM:MeOH) to afford the title compound (18 mg, 4% yield) as a white solid. ¹H NMR (300 MHz; CDCl₃): δ 7.16-7.35 (m, 4H), 6.82 (s, 1H), 6.48 (s, 1H), 6.36 (s, 1H), 3.50 (s, 2H), 3.34 (s, 3H) ppm. m/z 371 (M+H⁺).

### Example 30

### Synthesis of 2-[(4-cyano-1-methylisoquinolin-3-yl)amino]-N-(4-fluorophenyl)-N-methylacetamide

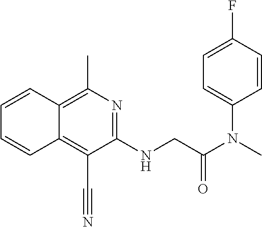 [SMILES: Cc1nc(NCC(=O)N(C)c2ccc(F)cc2)c(C#N)c2ccccc12]

### Step 1. Preparation of 1-methylisoquinolin-3-amine

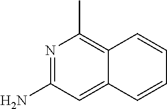

To a solution of 1-bromoisoquinolin-3-amine (5.0 g, 22.0 mmol) and methylboronic acid (2.6 g, 43.9 mmol) in 1,4-dioxane (50 mL) was added K₃PO₄ (9.3 g, 43. mmol) and Pd(dtbpf)Cl₂ (1.4 g, 2.2 mmol). The mixture was stirred overnight at 80° C. and then diluted with water and extracted with EtOAc. The organic layers were concentrated under reduced pressure and the residue was purified using silica gel chromatography (eluent: 3% EtOAc in PE) to afford 1-methylisoquinolin-3-amine (1.6 g, 45% yield) as a yellow solid.

### Step 2. Preparation of 4-bromo-1-methylisoquinolin-3-amine

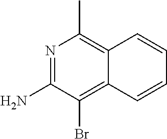

To a solution of 1-methylisoquinolin-3-amine (1.2 g, 7.7 mmol) in CH₃CN (13 mL) was added N-bromosuccinimide (1.6 g, 9.0 mmol) portion wise at 0° C. under nitrogen. The mixture was stirred overnight at room temperature under nitrogen and then concentrated under reduced pressure. The residue was purified using silica gel chromatography (eluent: 1% MeOH in DCM) to afford 200 mg of 4-bromo-1-methylisoquinolin-3-amine as a solid.

### Step 3. Preparation of 3-amino-1-methylisoquinoline-4-carbonitrile

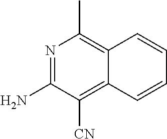

To a solution of 4-bromo-1-methylisoquinolin-3-amine (100 mg, 0.42 mmol) and Zn(CN)₂ (99 mg, 0.84 mmol) in DMF (1 mL) was added Pd(PPh₃)₄ (49 mg, 0.042 mmol). The mixture was irradiated with microwave radiation for 1 h at 200° C. The mixture cooled to room temperature and then diluted with water and extracted with EtOAc. The mixture was concentrated under reduced pressure and the residue was purified by Prep-TLC (40:1, DCM:MeOH) to afford 3-amino-1-methylisoquinoline-4-carbonitrile as a solid.

### Step 4. Preparation of 2-chloro-N-(4-fluorophenyl)-N-methylacetamide

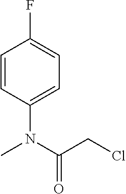

To a solution of 4-fluoro-N-methylaniline (1 g, 8 mmol) in DCM (10 mL) was added 2-chloroacetyl chloride (1.1 g, 9.6 mmol). Et₃N (1.6 g, 16 mmol) was added dropwise at 0° C. The mixture was stirred for 4 h at 0° C. The mixture was washed with water and the aqueous layer was extracted with DCM. The combined organic layers were concentrated under reduced pressure and the residue was purified using silica gel chromatography (eluent: 3% EtOAc in PE) to afford the title compound (1.3 g, 81% yield) as a yellow oil.

### Step 5. Preparation of 2-[(4-cyano-1-methylisoquinolin-3-yl)amino]-N-(4-fluorophenyl)-N-methylacetamide

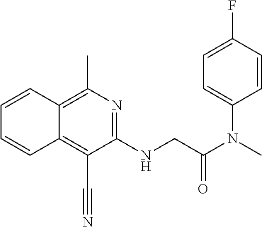 [SMILES: Cc1nc(NCC(=O)N(C)c2ccc(F)cc2)c(C#N)c2ccccc12]

To a solution of 3-amino-1-methylisoquinoline-4-carbonitrile (40 mg, 0.22 mmol, Example 32, Step 3) and 2-chloro-N-(4-fluorophenyl)-N-methylacetamide (88 mg, 0.44 mmol, Example 32, Step 4) in DMF (0.4 mL) was added K₂CO₃ (60 mg, 0.44 mmol) at room temperature. The mixture was heated to 80° C. for 3 h and then diluted with water and extracted with EtOAc. The residue was purified by Prep-TLC (20:1, DCM:MeOH) to afford the title compound (6 mg, 8% yield) as a yellow solid. ¹H NMR (400 MHz, DMSO-d₆): δ 8.10 (d, 1H), 7.73-7.77 (m, 1H), 7.66 (d, 1H), 7.49-7.53 (m, 2H), 7.35-7.39 (m, 3H), 7.22-7.23 (m, 1H), 3.90-3.98 (m, 2H), 3.19 (s, 3H), 2.80 (s, 3H) ppm. m/z 349 (M+H⁺).

### Example 31

### Synthesis of 2-[[5-chloro-2-cyano-3-(trifluoromethyl)phenyl]amino]-N-(4-fluorophenyl)-N-methylacetamide

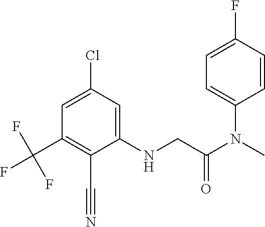 [SMILES: CN(C(=O)CNc1cc(Cl)cc(C(F)(F)F)c1C#N)c1ccc(F)cc1]

### Step 1. Preparation of 2-bromo-4-chloro-6-(trifluoromethyl)aniline

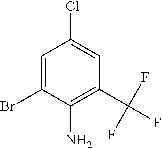 [SMILES: Nc1c(Br)cc(Cl)cc1C(F)(F)F]

To a solution of 4-chloro-2-(trifluoromethyl)aniline (1.0 g, 5.1 mmol) in DCM (10 mL) was added N-bromosuccinimide (0.9 g, 5.1 mmol) at 0° C. The mixture was stirred for 3 h at 0° C., washed with water and the organic layer was concentrated under reduced pressure. The residue was purified using silica gel chromatography (eluent: 2% EtOAc in PE) to afford 2-bromo-4-chloro-6-(trifluoromethyl)aniline (1.2 g, 86% yield) as a yellow solid.

### Step 2. Preparation of 2-bromo-4-chloro-6-(trifluoromethyl)benzonitrile

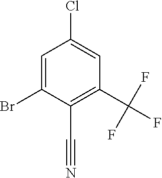 [SMILES: N#Cc1c(Br)cc(Cl)cc1C(F)(F)F]

To a solution of 2-bromo-4-chloro-6-(trifluoromethyl)aniline (1 g, 3.6 mmol) in MeCN (10 mL) was added CuCN (0.75 g, 8.4 mmol) and t-BuONO (1.1 g, 10.9 mmol). The mixture was stirred overnight at room temperature and then quenched with sat. NaHCO₃ and extracted with EtOAc. The combined organic layers were concentrated under reduced pressure and the residue was purified using silica gel chromatography (eluent: 10% EtOAc in PE) to give a residue. The residue was further purified by Prep-TLC (10:1, PE:EtOAc) to afford the title compound (200 mg, 19% yield) as a yellow solid.

### Step 3. Preparation of 2-[[5-chloro-2-cyano-3-(trifluoromethyl)phenyl]amino]-N-(4-fluorophenyl)-N-methylacetamide

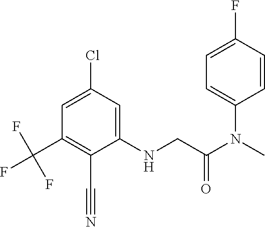 [SMILES: CN(C(=O)CNc1cc(Cl)cc(C(F)(F)F)c1C#N)c1ccc(F)cc1]

The title compound was prepared using General Procedure D employing 2-bromo-4-chloro-6-(trifluoromethyl)benzonitrile and 2-amino-N-(4-fluorophenyl)-N-methylacetamide TFA salt (Example 19, Step 2). The aqueous layer was extracted with EtOAc and the combined organic layers were concentrated under reduced pressure. The residue was purified by Prep-TLC (150:1, DCM:MeOH) to afford the title compound (96 mg) as a white solid. ¹H NMR (300 MHz, CDCl₃): δ 3.35 (s, 3H), 3.61 (d, 2H), 6.19 (s, 1H), 6.44 (s, 1H), 6.96 (s, 1H), 7.20-7.39 (m, 4H) ppm. m/z 386 (M+H⁺).

### Example 32

### Synthesis of 2-(3-cyano-4,6-bis(trifluoromethyl)pyridin-2-ylamino)-N-(4-fluorophenyl)-N-methylacetamide

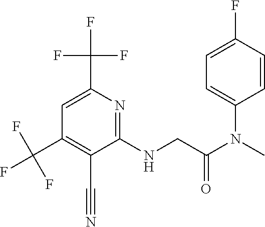 [SMILES: CN(C(=O)CNc1nc(C(F)(F)F)cc(C(F)(F)F)c1C#N)c1ccc(F)cc1]

The title compound was prepared using General Procedure B employing 2-amino-N-(4-fluorophenyl)-N-methylacetamide TFA salt (Example 21, Step 2) and 2-chloro-4,6-bis(trifluoromethyl) pyridine-3-carbonitrile (Intermediate A). The mixture was stirred overnight at room temperature and then diluted with EtOAc and washed with water. The organic layer was concentrated under reduced pressure and the residue was purified by Prep-TLC (2:1, EtOAc:hexanes) to afford 86 mg (56% yield) of the title compound as a white solid. ¹H NMR (400 MHz, DMSO-d₆): δ 3.16 (s, 3H), 3.82-3.95 (m, 2H), 7.26-7.44 (m, 5H), 8.33 (s, 1H) ppm. m z 421 (M+H⁺).

### Example 33

### Synthesis of 2-[[3-cyano-4,6-bis(trifluoromethyl)pyridin-2-yl]amino]-N-(4-methoxyphenyl)-N-methylacetamide

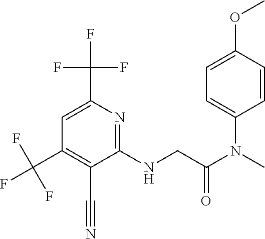 [SMILES: COc1ccc(N(C)C(=O)CNc2nc(C(F)(F)F)cc(C(F)(F)F)c2C#N)cc1]

### Step 1. Preparation of tert-butyl N-[[(4-methoxyphenyl)(methyl)carbamoyl]methyl]carbamate

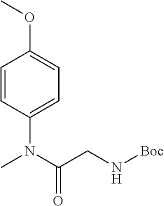 [SMILES: COc1ccc(N(C)C(=O)CNC(=O)OC(C)(C)C)cc1]

To a solution of 2-[[(tert-butoxy)carbonyl]amino]acetic acid (900 mg, 5.1 mmol) and 4-methoxy-N-methylaniline (705 mg, 5.1 mmol) in THF (9 mL) were added DIEA (996 mg, 7.76 mmol), and propylphosphonic anhydride solution (3269 mg, 10 mmol, 50 wt % in EtOAc) at room temperature. The mixture was stirred overnight at 50° C. under nitrogen and diluted with EtOAc. The mixture was washed with water and then concentrated under reduced pressure. The residue was purified by Prep-TLC (150:1, DCM:MeOH) to afford tert-butyl N-[[(4-methoxyphenyl)(methyl)carbamoyl]methyl]carbamate (900 mg, 60% yield) as a yellow solid.

### Step 2. Preparation of 2-amino-N-(4-methoxyphenyl)-N-methylacetamide TFA salt

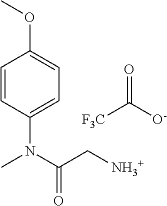

To a solution of tert-butyl N-[[(4-methoxyphenyl)(methyl)carbamoyl]methyl]carbamate (900 mg, 3.1 mmol) in DCM (9 mL) was added TFA (1.8 mL) at room temperature. The mixture was stirred overnight at room temperature and then concentrated under reduced pressure. The residue was purified by Prep-TLC (20:1, DCM:MeOH) to afford the title compound (200 mg, 34% yield) as a yellow oil.

### Step 3. Preparation of 2-[[3-cyano-4,6-bis(trifluoromethyl)pyridin-2-yl]amino]-N-(4-methoxyphenyl)-N-methylacetamide

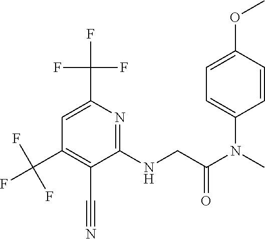 [SMILES: COc1ccc(N(C)C(=O)CNc2nc(C(F)(F)F)cc(C(F)(F)F)c2C#N)cc1]

The title compound was prepared using General Procedure B employing 2-amino-N-(4-methoxyphenyl)-N-methylacetamide TFA salt and 2-chloro-4,6-bis(trifluoromethyl)pyridine-3-carbonitrile (Intermediate A). The mixture was stirred overnight at 50° C. and then diluted with EtOAc, washed with water and concentrated under reduced pressure. The residue was purified by Prep-TLC (200:1, DCM:MeOH) to afford the title compound (57 mg) as a white solid. ¹H NMR (300 MHz; DMSO-d₆): δ 3.14 (s, 3H), 3.79-3.84 (m, 5H), 7.02-7.05 (m, 2H), 7.30-7.33 (m, 2H), 7.45 (s, 1H), 8.29 (s, 1H). m/z 433 (M+H⁺).

### Example 34

### Synthesis of (S)-2-(5-chloro-3-cyano-4,6-dimethylpyridin-2-ylamino)-N1-(4-fluorophenyl)-N1-methylsuccinamide

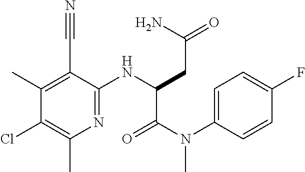

### Step 1. Preparation of (S)-methyl 3-(benzyloxycarbonylamino)-4-((4-fluorophenyl)-(methyl)amino)-4-oxobutanoate

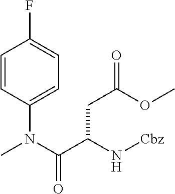

To a solution of (2S)-2-[[(benzyloxy)carbonyl]amino]-4-methoxy-4-oxobutanoic acid (1 g, 3.6 mmol) and 4-fluoro-N-methylaniline (0.44 g, 3.6 mmol) in THE (10 mL) was added propylphosphonic anhydride solution (3.4 g, 5.343 mmol, 50 wt % in EtOAc), and DIEA (0.92 g, 7.111 mmol) under nitrogen. The mixture was stirred for 4 h at 50° C. and then diluted with water and extracted with EtOAc. The mixture was concentrated under reduced pressure and the residue was purified using silica gel chromatography (eluent: 17% EtOAc in PE) to give 0.9 g of the title compound as a light-yellow oil.

### Step 2. Preparation of(S)-3-(benzyloxycarbonylamino)-4-((4-fluorophenyl)(methyl)amino)-4-oxobutanoic acid

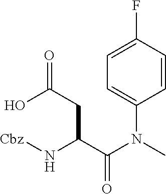 [SMILES: CN(C(=O)[C@H](CC(=O)O)NC(=O)OCc1ccccc1)c1ccc(F)cc1]

To a solution of methyl 3-[[(benzyloxy)carbonyl]amino]-3-[(4-fluorophenyl)(methyl)-carbamoyl] propanoate (600 mg, 1.5 mmol) in MeOH (6 mL) was added a solution of 1 M LiOH (4.64 mL, 4.64 mmol) at room temperature. The mixture was stirred at room temperature for 5.5 h under nitrogen. The mixture was diluted with water and extracted with EtOAc. The aqueous layer was acidified by 1 M HCl to pH 2. The solid was collected by filtration and the solid was washed with water. The solid was dried under vacuum to afford the title compound (445 mg, 77% yield) as a white solid.

### Step 3. Preparation of (S)-benzyl 4-amino-1-((4-fluorophenyl)(methyl)amino)-1,4-dioxobutan-2-ylcarbamate

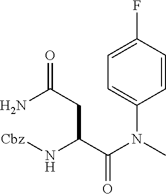 [SMILES: CN(C(=O)[C@H](CC(N)=O)NC(=O)OCc1ccccc1)c1ccc(F)cc1]

To a solution of 3-[[(benzyloxy)carbonyl]amino]-3-[(4-fluorophenyl)(methyl)-carbamoyl]propanoic acid (385 mg, 1.0 mmol) in DMF (8 mL) was added NH₄Cl (165 mg, 3.1 mmol), DIEA (665 mg, 5.1 mmol) and HATU (391 mg, 1.0 mmol) at room temperature. The mixture was stirred for 11 h at 50° C. under nitrogen and then cooled to room temperature and diluted with water. The mixture was extracted with EtOAc and the combined organic layers were washed with brine and concentrated under reduced pressure. The residue was purified using silica gel chromatography (eluent: 5% MeOH in DCM) to give the title compound (353 mg, 92% yield) as colorless oil.

### Step 4. Preparation of (S)-2-amino-N1-(4-fluorophenyl)-N1-methylsuccinamide

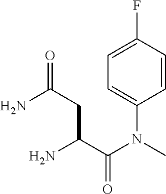 [SMILES: CN(C(=O)[C@@H](N)CC(N)=O)c1ccc(F)cc1]

To a solution of benzyl (S)-benzyl 4-amino-1-((4-fluorophenyl)(methyl)amino)-1,4-dioxobutan-2-ylcarbamate (353 mg, 0.95 mmol) in MeOH (10 mL) was added 10% Pd/C (37 mg) at room temperature under nitrogen. The flask was evacuated and back filled with hydrogen and the mixture was stirred at room temperature under hydrogen for 12 h. The mixture was filtered and the filtrate was concentrated under reduced pressure. The residue was purified using silica gel chromatography (eluent: 5% MeOH in DCM) to afford the title compound (220 mg, 97% yield) as a colorless oil.

### Step 5. Preparation of (S)-2-(5-chloro-3-cyano-4,6-dimethylpyridin-2-ylamino)-N1-(4-fluoro-phenyl)-N1-methylsuccinamide

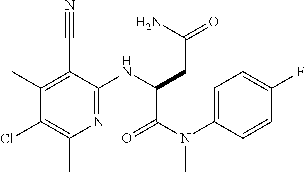

The title compound was prepared using General Procedure B employing (S)-2-amino-N1-(4-fluorophenyl)-N1-methylsuccinamide (170 mg, 0.71 mmol) and 2,5-dichloro-4,6-dimethyl-pyridine-3-carbonitrile. The mixture was heated to 100° C. for 20 h and then cooled to room temperature, diluted with water and extracted with EtOAc. The combined organic layers were concentrated under reduced pressure and the residue was purified by prep-TLC (10:1, DCM:MeOH) to afford the title compound (112 mg, 49% yield) as an off-white solid. ¹H NMR (300 MHz; DMSO-d₆): δ 7.49-7.53 (m, 2H), 7.24-7.36 (m, 3H), 7.00-7.08 (m, 1H), 6.87 (s, 1H), 4.89-4.96 (m, 1H), 3.13 (s, 3H), 2.38-2.45 (m, 8H) ppm. m/z 404 (M+H⁺).

### Example 35

### Synthesis of (S)-2-amino-N-(4-fluorophenyl)-3-hydroxy-N-methylpropanamide

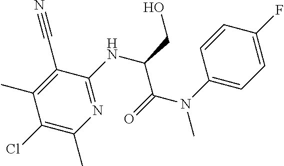 [SMILES: Cc1nc(N[C@@H](CO)C(=O)N(C)c2ccc(F)cc2)c(C#N)c(C)c1Cl]

### Step 1. Preparation of (S)-benzyl 1-((4-fluorophenyl)(methyl)amino)-3-hydroxy-1-oxopropan-2-ylcarbamate

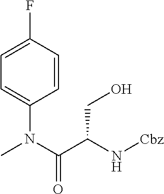 [SMILES: CN(C(=O)[C@H](CO)NC(=O)OCc1ccccc1)c1ccc(F)cc1]

The title compound was prepared using similar procedure as Example 34, Step 1 replacing (2S)-2-[[(benzyloxy)carbonyl]amino]-4-methoxy-4-oxobutanoic acid with (2S)-2-[[(benzyloxy)carbonyl]amino]-3-hydroxypropanoic acid. The mixture was stirred at room temperature for 17 h and then diluted with EtOAc and washed with water and then 0.1 M HCl. The organic layer was concentrated under reduced pressure and the residue was purified using silica gel chromatography (eluent: 2% MeOH in DCM) to afford the title compound (480 mg, 30% yield) as green oil.

### Step 2. Preparation of (S)-2-amino-N-(4-fluorophenyl)-3-hydroxy-N-methylpropanamide

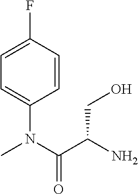

The title compound was prepared using a similar procedure as Example 34, Step 4 replacing (S)-benzyl 4-amino-1-((4-fluorophenyl)(methyl)amino)-1,4-dioxobutan-2-ylcarbamate with (S)-benzyl 1-((4-fluorophenyl)(methyl)amino)-3-hydroxy-1-oxopropan-2-ylcarbamate. The mixture was stirred for 15.5 h at room temperature under hydrogen atmosphere. The mixture was filtered and the filtrate was concentrated under reduced pressure to afford the title compound (283 mg, 96% yield) as green oil which was used without further purification.

### Step 3. Preparation of (S)-2-(5-chloro-3-cyano-4,6-dimethylpyridin-2-ylamino)-N-(4-fluorophenyl)-3-hydroxy-N-methylpropanamide

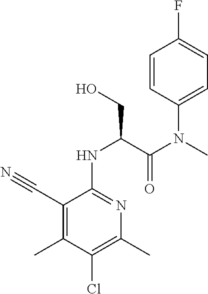 [SMILES: Cc1nc(N[C@@H](CO)C(=O)N(C)c2ccc(F)cc2)c(C#N)c(C)c1Cl]

The title compound was prepared using General Procedure D employing (S)-2-amino-N-(4-fluorophenyl)-3-hydroxy-N-methylpropanamide and 2,5-dichloro-4,6-dimethylpyridine-3-carbonitrile. The mixture was degassed with nitrogen for 3 minutes and then heated to 80° C. for 21 h. The mixture was cooled to room temperature, diluted with water and extracted with EtOAc. The combined organic layers were washed with brine and concentrated under reduced pressure. The residue was purified by prep-TLC (10: DCM:MeOH) to afford the title compound (6 mg, 8% yield) as an off-white solid. ¹H NMR (300 MHz; MeOD): δ 7.53-7.49 (m, 2H), 7.26-7.21 (m, 2H), 4.96-4.94 (m, 1H), 3.79-3.61 (m, 2H), 3.32 (s, 3H), 2.48 (s, 6H) ppm. m/z 377 (M+H⁺)

### Example 36

### Synthesis of (S)-2-(5-chloro-3-cyano-4,6-dimethylpyridin-2-ylamino)-N-(4-fluorophenyl)-3-(4-hydroxyphenyl)-N-methylpropanamide and (R)-2-(5-chloro-3-cyano-4,6-dimethylpyridin-2-ylamino)-N-(4-fluorophenyl)-3-(4-hydroxyphenyl)-N-methylpropanamide

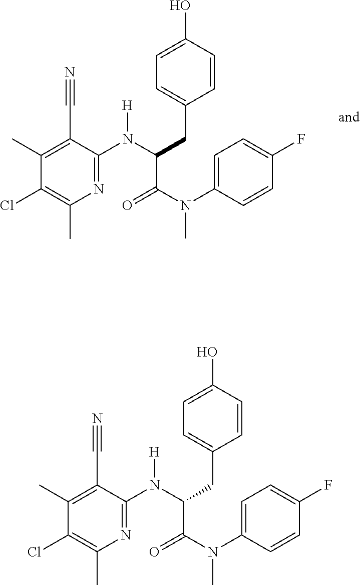

### Step 1. Preparation of (S)-2-[(5-chloro-3-cyano-4,6-dimethylpyridin-2-yl)amino]-3-(4-hydroxyphenyl)propanoic acid

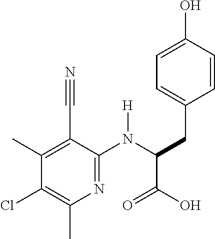

To a solution of 2,5-dichloro-4,6-dimethylpyridine-3-carbonitrile (3.00 g, 14.9 mmol) in DMSO (30 mL) was added (S)-2-amino-3-(4-hydroxyphenyl)propanoic acid (2.70 g, 14.9 mmol) and DBU (4.5 g, 29.8 mmol). The mixture was stirred overnight at 100° C. and then quenched by sat. NH₄Cl and extracted with EtOAc. The organic layer was concentrated under reduced pressure and the residue was purified using silica gel chromatography (eluent: 2% MeOH in DCM) to afford the title compound (900 mg, 17% yield) as a yellow solid.

### Step 2. Preparation of (S)-2-(5-chloro-3-cyano-4,6-dimethylpyridin-2-ylamino)-N-(4-fluorophenyl)-3-(4-hydroxyphenyl)-N-methylpropanamide and (R)-2-(5-chloro-3-cyano-4,6-dimethylpyridin-2-ylamino)-N-(4-fluorophenyl)-3-(4-hydroxyphenyl)-N-methylpropanamide

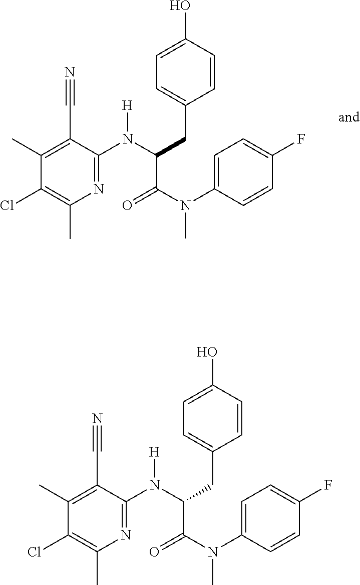

The title compounds were prepared using General Procedure A employing (S)-2-[(5-chloro-3-cyano-4,6-dimethylpyridin-2-yl)amino]-3-(4-hydroxyphenyl)propanoic acid and 4-fluoro-N-methylaniline. The mixture was stirred overnight at 70° C. A solution of sat. NH₄Cl was added and the mixture was extracted with EtOAc. The combined organic layers were concentrated under reduced pressure and the residue was purified by Prep-TLC (8:1, DCM:MeOH) to afford a 1:1 mixture of the title compounds (138 mg, 35% yield) as a white solid. ¹H NMR (300 MHz; DMSO-d₆): δ 9.14 (s, 1H), 7.42-7.49 (m, 2H), 7.28-7.40 (m, 2H), 6.60-6.74 (m, 3H), 6.51-6.55 (m, 2H), 4.60-4.72 (m, 1H), 3.14 (s, 3H), 2.72-2.89 (m, 2H), 2.32-2.36 (m, 6H) ppm. m/z 453 (M+H⁺).

### Example 37

### Synthesis of (S)-2-(5-chloro-3-cyano-4,6-dimethylpyridin-2-ylamino)-N-(4-fluorophenyl)-4-hydroxy-N-methylbutanamide

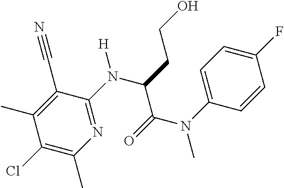

### Step 1. Preparation of (S)-benzyl 1-((4-fluorophenyl)(methyl)amino)-4-hydroxy-1-oxobutan-2-ylcarbamate

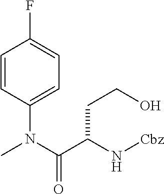 [SMILES: CN(C(=O)[C@H](CCO)NC(=O)OCc1ccccc1)c1ccc(F)cc1]

To a solution of (S)-3-(benzyloxycarbonylamino)-4-((4-fluorophenyl)(methyl)amino)-4-oxobutanoic acid (400 mg, 1.1 mmol, Example 34, Step 2) in THE (4 mL) was added borane dimethyl sulfide complex (0.20 mL, 2 mmol, 10 M) at 0° C. The mixture was stirred at room temperature for 2 h under nitrogen atmosphere and then was quenched with MeOH and concentrated under reduced pressure. The residue was purified using silica gel chromatography (eluent: 2% MeOH in DCM) to afford the title compound (82 mg, 21% yield) as a colorless oil.

### Step 2. Preparation of (S)-2-amino-N-(4-fluorophenyl)-4-hydroxy-N-methylbutanamide

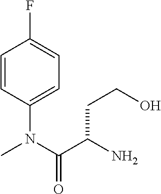 [SMILES: CN(C(=O)[C@@H](N)CCO)c1ccc(F)cc1]

The title compound was prepared using similar procedure as Example 34, Step 4 employing benzyl 1-((4-fluorophenyl)(methyl)amino)-4-hydroxy-1-oxobutan-2-ylcarbamate. The mixture was stirred at room temperature under hydrogen for 12 h and filtered and the filtrate was concentrated under reduced pressure. The residue was purified by prep-TLC (5:1, DCM:MeOH) to afford the title compound (52 mg, 83% yield) as a colorless oil.

### Step 3. Preparation of (S)-2-(5-chloro-3-cyano-4,6-dimethylpyridin-2-ylamino)-N-(4-fluorophenyl)-4-hydroxy-N-methylbutanamide

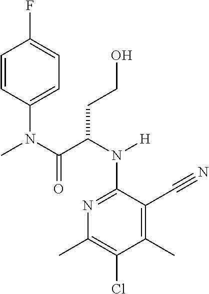 [SMILES: Cc1nc(N[C@@H](CCO)C(=O)N(C)c2ccc(F)cc2)c(C#N)c(C)c1Cl]

The title compound was prepared using General Procedure B employing (S)-2-amino-N-(4-fluorophenyl)-4-hydroxy-N-methylbutanamide and 2,5-dichloro-4,6-dimethylpyridine-3-carbonitrile. The mixture was heated to 100° C. for 21 h under nitrogen. The mixture was cooled to room temperature, diluted with EtOAc and washed with water. The organic layer was concentrated under reduced pressure. The residue was purified by prep-TLC (20:1, DCM:MeOH) to afford the title compound (33 mg, 46% yield) as an off-white solid. H NMR (300 MHz; DMSO-d₆): δ 7.52-7.58 (m, 2H), 7.26-7.39 (m, 2H), 7.00-7.06 (m, 1H), 4.61-4.68 (m, 2H), 3.14 (s, 3H), 2.41-2.46 (m, 6H), 1.70-1.78 (m, 2H) ppm. m/z 391 (M+H⁺).

### Example 38

### Synthesis of 2-(3-cyano-4,6-bis(trifluoromethyl)pyridin-2-ylamino)-N-(4-fluorophenyl)-N-(2,2,2-trifluoroethyl)acetamide

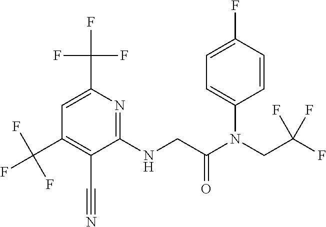 [SMILES: N#Cc1c(C(F)(F)F)cc(C(F)(F)F)nc1NCC(=O)N(CC(F)(F)F)c1ccc(F)cc1]

### Step 1. Preparation of 2,2,2-trifluoro-N-(4-fluorophenyl)acetamide

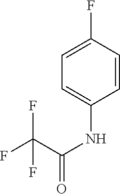 [SMILES: O=C(Nc1ccc(F)cc1)C(F)(F)F]

To a solution of 4-fluoroaniline (1.0 g, 9 mmol) in THE (10 mL) was added TFAA (1.9 g, 9 mmol) and Et₃N (1.8 g, 18 mmol) at 0° C. The mixture was stirred for 2 h at 0° C. under nitrogen and then diluted with EtOAc. The mixture was washed with water and the organic layer was concentrated under reduced pressure. The residue was purified using silica gel chromatography (eluent: 17% EtOAc in PE) to afford the title compound (1.0 g, 55% yield) as a yellow oil.

### Step 2. Preparation of 4-fluoro-N-(2,2,2-trifluoroethyl)aniline

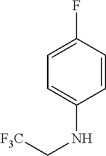 [SMILES: Fc1ccc(NCC(F)(F)F)cc1]

To a solution of 2,2,2-trifluoro-N-(4-fluorophenyl)acetamide (1 g, 4.8 mmol) in THE (10 mL) was added LiAlH₄ (183 mg, 4.8 mmol). The mixture was stirred overnight at 70° C. under nitrogen and then diluted with EtOAc. The mixture was washed with water and the aqueous layer was extracted with EtOAc. The combined organic layers were concentrated under reduced pressure and the residue was purified using silica gel chromatography (eluent: 9% EtOAc in hexanes) to afford 4-fluoro-N-(2,2,2-trifluoroethyl)aniline (800 mg, 86% yield) as a yellow oil.

### Step 3. Preparation of 2-chloro-N-(4-fluorophenyl)-N-(2,2,2-trifluoroethyl)acetamide

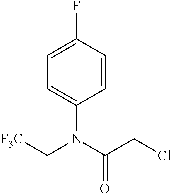 [SMILES: O=C(CCl)N(CC(F)(F)F)c1ccc(F)cc1]

To a solution of 4-fluoro-N-(2,2,2 trifluoroethyl)aniline (600 mg, 3.1 mmol) in DCM (6 mL) was added chloroacetyl chloride (526 mg, 4.7 mmol) and Et₃N (629 mg, 6.2 mmol) at 0° C. The mixture was stirred for 1 h at 0° C. under nitrogen. The mixture was diluted with water and extracted with EtOAc. The combined organic layers were concentrated under reduced pressure. The residue was purified by Prep-TLC (50:1, DCM:MeOH) to afford 2-chloro-N-(4-fluorophenyl)-N-(2,2,2 trifluoroethyl)acetamide (475 mg, 57% yield) as a yellow solid.

### Step 4. Preparation of 2-amino-N-(4-fluorophenyl)-N-(2,2,2-trifluoroethyl)acetamide

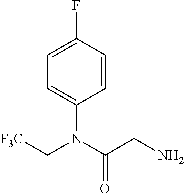 [SMILES: NCC(=O)N(CC(F)(F)F)c1ccc(F)cc1]

2-Chloro-N-(4-fluorophenyl)-N-(2,2,2-trifluoroethyl)acetamide (475 mg, 1.8 mmol) was dissolved in NH₃ in MeOH (25 mL, 8 M). The mixture was stirred overnight at 60° C. and then concentrated under reduced pressure. The residue was purified by Prep-TLC (10:1, DCM:MeOH) to afford the title compound (300 mg, 68% yield) as a white solid.

### Step 5. Preparation of 2-(3-cyano-4,6-bis(trifluoromethyl)pyridin-2-ylamino)-N-(4-fluorophenyl)-N-(2,2,2-trifluoroethyl)acetamide

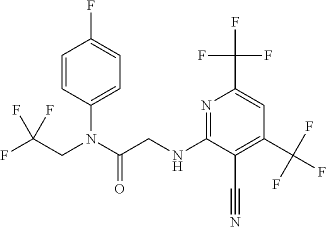 [SMILES: N#Cc1c(C(F)(F)F)cc(C(F)(F)F)nc1NCC(=O)N(CC(F)(F)F)c1ccc(F)cc1]

The title compound was prepared using General Procedure B employing 2-amino-N-(4-fluorophenyl)-N-(2,2,2-trifluoroethyl)acetamide and 2-chloro-4,6-bis(trifluoromethyl)pyridine-3-carbonitrile (Intermediate A). The mixture was stirred overnight at room temperature and then diluted with EtOAc and washed with water. The organic layer was concentrated under reduced pressure and the residue was purified by Prep-TLC (50:1, DCM:MeOH) to afford the title compound (53 mg) as a white solid. ¹H NMR (300 MHz; DMSO-d₆): δ 8.49-8.52 (m, 1H), 7.48-7.53 (m, 3H), 7.33-7.42 (m, 2H), 4.41-4.52 (m, 2H), 3.83 (d, 2H) ppm. m/z 487 (M−H⁻).

### Example 39

### Synthesis of 2-((4,6-bis(trifluoromethyl)pyridin-2-yl)amino)-N-(4-fluorophenyl)-N-methylacetamide

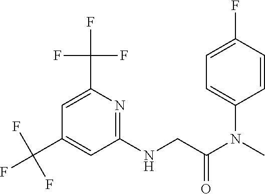 [SMILES: CN(C(=O)CNc1cc(C(F)(F)F)cc(C(F)(F)F)n1)c1ccc(F)cc1]

The title compound was prepared using General Procedure B employing 2-chloro-4,6-bis(trifluoromethyl)pyridine and N-(4-fluorophenyl)-N-methylacetamide. The mixture was diluted with water and extracted with EtOAc. The combined organic layer was concentrated under reduced pressure and the residue was purified by silica gel chromatography (0-100% EtOAc in hexanes) to afford 154 mg (47% yield) of the title compound as a white solid. ¹H NMR (400 MHz, DMSO-d₆) δ 7.91 (s, 1H), 7.56-7.37 (m, 2H), 7.37-7.18 (m, 4H), 3.83 (s, 2H), 3.15 (s, 3H). m/z 396 (M+H⁺).

### Example 40

### Synthesis of 2-((3-cyano-4,6-bis(trifluoromethyl)pyridin-2-yl)amino)-N-methyl-N-(4-(trifluoromethoxy)phenyl)acetamide

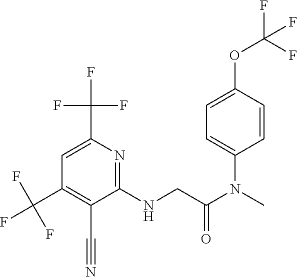 [SMILES: CN(C(=O)CNc1nc(C(F)(F)F)cc(C(F)(F)F)c1C#N)c1ccc(OC(F)(F)F)cc1]

### Step 1. Preparation of tert-butyl (2-(methyl(4-(trifluoromethoxy)phenyl)amino)-2-oxoethyl)carbamate

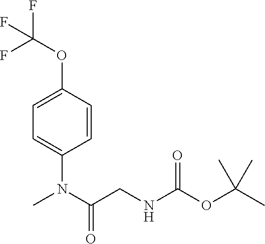 [SMILES: CN(C(=O)CNC(=O)OC(C)(C)C)c1ccc(OC(F)(F)F)cc1]

The title compound was prepared using General Procedure A with N-methyl-4-(trifluoromethoxy)aniline and N-Boc glycine. The residue was purified using silica gel chromatography (eluent: 0-75% EtOAc in hexanes) to afford 346 mg (63% yield) of tert-butyl (2-(methyl(4-(trifluoromethoxy)phenyl)amino)-2-oxoethyl)carbamate as a white solid.

### Step 2. Preparation of 2-amino-N-methyl-N-(4-(trifluoromethoxy)phenyl)acetamide hydrochloride

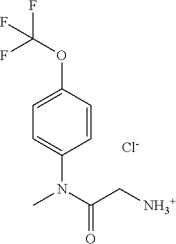 [SMILES: CN(C(=O)C[NH3+])c1ccc(OC(F)(F)F)cc1 ; [Cl-]]

To solution of tert-butyl (2-(methyl(4-(trifluoromethoxy)phenyl)amino)-2-oxoethyl)carbamate (346 mg, 1.5 mmol) in EtOAc (2 mL) at room temperature was added a solution of HCl in 1,4-dioxane (4.0 M, 2 mL). The mixture was stirred at room temperature for 3 h and a white precipitate formed. The mixture was concentrated under reduced pressure and the residue was used without further purification.

### Step 4. Preparation of 2-((3-cyano-4,6-bis(trifluoromethyl)pyridin-2-yl)amino)-N-methyl-N-(4-(trifluoromethoxy)phenyl)acetamide

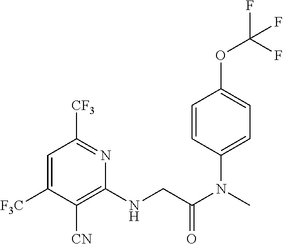 [SMILES: CN(C(=O)CNc1nc(C(F)(F)F)cc(C(F)(F)F)c1C#N)c1ccc(OC(F)(F)F)cc1]

The title compound was prepared using General Procedure B employing 2-amino-N-methyl-N-(4-(trifluoromethoxy)phenyl)acetamide hydrochloride and 2-chloro-4,6-bis(trifluoro-methyl)pyridine-3-carbonitrile (Intermediate A). The mixture was diluted with water and extracted with EtOAc and the combined organic layers were concentrated under reduced pressure. The residue was purified using silica gel chromatography (eluent: 0-100% EtOAc in hexanes) to afford 95 mg (49%) of the title compound as a white solid. ¹H NMR (400 MHz, CDCl₃) δ 7.35 (s, 4H), 7.18 (s, 1H), 6.61 (s, 1H), 3.98 (s, 2H), 3.33 (s, 3H). m/z 487 (M+H⁺).

### Example 41

### Synthesis of 2-((3-cyano-4,6-bis(trifluoromethyl)pyridin-2-yl)amino)-N-(4-cyclopropoxyphenyl)-N-methylacetamide

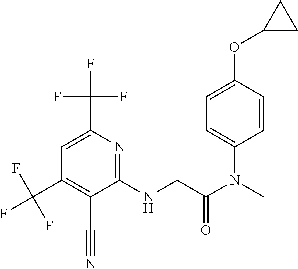 [SMILES: CN(C(=O)CNc1nc(C(F)(F)F)cc(C(F)(F)F)c1C#N)c1ccc(OC2CC2)cc1]

### Step 1. Preparation of tert-butyl (2-((4-cyclopropoxyphenyl)(methyl)amino)-2-oxoethyl)carbamate

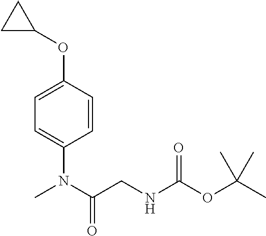 [SMILES: CN(C(=O)CNC(=O)OC(C)(C)C)c1ccc(OC2CC2)cc1]

The title compound was prepared using General Procedure A with 4-cyclopropoxy-N-methylaniline and N-Boc glycine. The residue was purified by flash chromatography (silica gel) with (0-75% EtOAc/Hexanes) to afford 140 mg (92% yield) of the title compound as a white solid.

### Step 2. Preparation of 2-amino-N-(4-cyclopropoxyphenyl)-N-methylacetamide hydrochloride

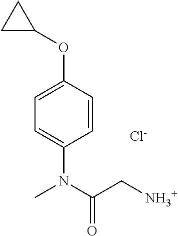 [SMILES: CN(C(=O)C[NH3+])c1ccc(OC2CC2)cc1 ; [Cl-]]

To a solution of tert-butyl (2-((4-cyclopropoxyphenyl)(methyl)amino)-2-oxoethyl)-carbamate in EtOAc (2 mL) at room temperature was added a solution of HCl in 1,4-dioxane (4.0 M, 2 mL). The mixture was stirred at room temperature for 3 h and a white precipitate formed. The mixture was concentrated under reduced pressure and the residue was used without further purification.

### Step 4. Preparation of 2-((3-cyano-4,6-bis(trifluoromethyl)pyridin-2-yl)amino)-N-(4-cyclopropoxyphenyl)-N-methylacetamide

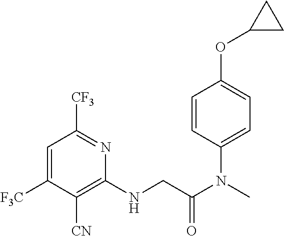 [SMILES: CN(C(=O)CNc1nc(C(F)(F)F)cc(C(F)(F)F)c1C#N)c1ccc(OC2CC2)cc1]

The title compound was prepared using General Procedure B employing 2-amino-N-(4-cyclopropoxyphenyl)-N-methylacetamide hydrochloride and 2-chloro-4,6-bis(trifluoromethyl) pyridine-3-carbonitrile (Intermediate A). The mixture was diluted with water and extracted with EtOAc and the combined organic layers were concentrated under reduced pressure. The residue was purified by flash chromatography (silica gel, 0-100% EtOAc/Hexanes) to afford 81 mg (56%) of the title compound as a white solid. ¹H NMR (400 MHz, CDCl₃) δ 7.21-7.10 (m, 5H), 6.68 (s, 1H), 3.97 (s, 2H), 3.85-3.70 (m, 1H), 3.30 (s, 3H), 0.92-0.78 (m, 4H) ppm. m/z 459 (M+H⁺).

### Example 42

### Synthesis of 2-((3-cyano-4,6-bis(trifluoromethyl)pyridin-2-yl)amino)-N-cyclopropyl-N-(4-fluorophenyl)acetamide

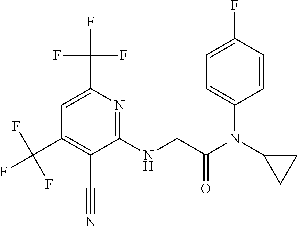 [SMILES: N#Cc1c(C(F)(F)F)cc(C(F)(F)F)nc1NCC(=O)N(c1ccc(F)cc1)C1CC1]

### Step 1. Preparation of N-cyclopropyl-4-fluorobenzenamine

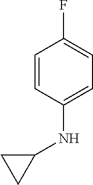 [SMILES: Fc1ccc(NC2CC2)cc1]

To a solution of 1-bromo-4-fluorobenzene (4.0 g, 23 mmol) in THE (40 mL) was added cyclopropanamine (1.6 g, 27.4 mmol), (tert-butoxy)sodium (2.6 g, 27.4 mmol), and t-BuBrettphospdallyoTf (DeAngelis A. J., et al., Journal of Organic Chemistry (2015); 80: 6794-6813) (178 mg, 0.23 mmol) under nitrogen. The mixture was stirred for 12 h at 50° C. under nitrogen and then diluted with water and extracted EtOAc. The combined organic layers were dried over Na₂SO₄ and concentrated under reduced pressure. The residue was purified using silica gel chromatography (eluent: 9% EtOAc in hexanes) to afford 2.9 g of N-cyclopropyl-4-fluorobenzenamine as oily liquid.

### Step 2. Preparation of 2-chloro-N-cyclopropyl-N-(4-fluorophenyl)acetamide

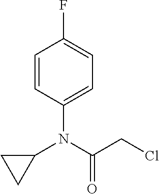 [SMILES: O=C(CCl)N(c1ccc(F)cc1)C1CC1]

To a solution of N-cyclopropyl-4-fluoroaniline (1.0 g, 6.6 mmol) in DCM (15 mL) was added 2-chloroacetyl chloride (0.82 g, 7.3 mmol) and Et₃N (1.3 g, 13.3 mmol) under nitrogen. The mixture was stirred for 2 h at room temperature under nitrogen and then diluted with water and extracted with EtOAc. The combined organic layers were dried over anhydrous Na₂SO₄ and concentrated under reduced pressure and the residue was purified using silica gel chromatography (eluent: 5% EtOAc in hexanes) to afford 500 mg of the title compound as an oily liquid.

### Step 3. Preparation of 2-((3-cyano-4,6-bis(trifluoromethyl)pyridin-2-yl)amino)-N-cyclopropyl-N-(4-fluorophenyl)acetamide

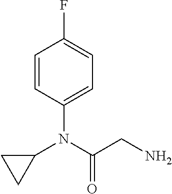 [SMILES: NCC(=O)N(c1ccc(F)cc1)C1CC1]

2-Chloro-N-cyclopropyl-N-(4-fluorophenyl)acetamide (500 mg, 2.2 mmo) was dissolved in NH₃ in MeOH (50 mL, 8 M) under nitrogen. The mixture was stirred for 12 h at room temperature and then concentrated under reduced pressure. The residue was purified using silica gel chromatography (eluent: 9% MeOH in DCM) to afford 180 mg of the title compound as an oily liquid.

### Step 4. Preparation of 2-((3-cyano-4,6-bis(trifluoromethyl)pyridin-2-yl)amino)-N-cyclopropyl-N-(4-fluorophenyl)acetamide

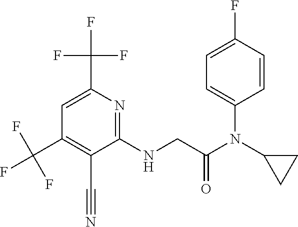 [SMILES: N#Cc1c(C(F)(F)F)cc(C(F)(F)F)nc1NCC(=O)N(c1ccc(F)cc1)C1CC1]

The title compound was prepared using General Procedure B employing 2-amino-N-cyclopropyl-N-(4-fluorophenyl)acetamide and 2-chloro-4,6-bis(trifluoromethyl)pyridine-3-carbonitrile (Intermediate A). The mixture was stirred for 12 h at 100° C. under nitrogen and then diluted with water and extracted with EtOAc. The combined organic layers were dried over anhydrous Na₂SO₄ and concentrated under reduced pressure and the residue was purified by Prep-TLC (100:1, DCM:MeOH) to afford 97 mg of the title compound as a white solid. ¹H NMR (400 MHz; DMSO-d₆): δ 8.44 (br s, 1H), 7.47 (s, 1H), 7.20-7.28 (m, 4H), 4.00-4.80 (m, 2H), 3.12-3.20 (m, 1H), 0.70-0.92 (m, 2H), 0.42-0.58 (m, 2H) ppm. m/z 447 (M+H⁺)

### Example 43

### Synthesis of 2-((3-cyano-4,6-bis(trifluoromethyl)pyridin-2-yl)amino)-N-(4-cyanophenyl)-N-methylacetamide

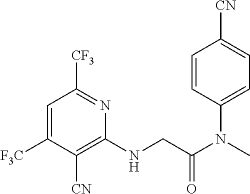 [SMILES: CN(C(=O)CNc1nc(C(F)(F)F)cc(C(F)(F)F)c1C#N)c1ccc(C#N)cc1]

### Step 1. Preparation of tert-butyl (2-((4-cyanophenyl)(methyl)amino)-2-oxoethyl)carbamate

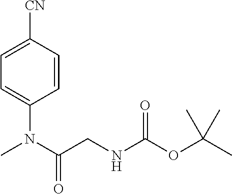 [SMILES: CN(C(=O)CNC(=O)OC(C)(C)C)c1ccc(C#N)cc1]

The title compound was prepared using General Procedure A with 4-(methylamino)benzonitrile and N-Boc glycine. The residue was purified using silica gel chromatography (eluent: 0-75% EtOAc in hexanes) to afford 140 mg (43% yield) of tert-butyl (2-((4-cyanophenyl)(methyl)amino)-2-oxoethyl)carbamate as a white solid.

### Step 2. Preparation of 2-amino-N-(4-cyanophenyl)-N-methylacetamide hydrochloride

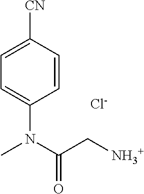

To a solution of tert-butyl (2-((4-cyanophenyl)(methyl)amino)-2-oxoethyl)carbamate in EtOAc (2 mL) at room temperature was added a solution of HCl in 1,4-dioxane (4.0 M, 2 mL). The mixture was stirred at room temperature for 3 h and a white precipitate formed. The mixture was concentrated under reduced pressure and the residue was used without further purification.

### Step 4. Preparation of 2-((3-cyano-4,6-bis(trifluoromethyl)pyridin-2-yl)amino)-N-(4-cyanophenyl)-N-methylacetamide

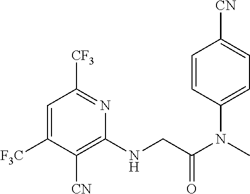 [SMILES: CN(C(=O)CNc1nc(C(F)(F)F)cc(C(F)(F)F)c1C#N)c1ccc(C#N)cc1]

The title compound was prepared using General Procedure B employing 2-amino-N-(4-cyanophenyl)-N-methylacetamide hydrochloride and 2-chloro-4,6-bis(trifluoromethyl) pyridine-3-carbonitrile (Intermediate A). The mixture was diluted with water and extracted with EtOAc and the combined organic layers were concentrated under reduced pressure. The residue was purified using silica gel chromatography (eluent: 0-100% EtOAc in hexanes) to afford 36 mg (32%) of the title compound as a white solid. ¹H NMR (400 MHz, CDCl₃) δ 7.80 (d, 2H), 7.45 (d, 2H), 7.21 (s, 1H), 6.65 (s, 1H), 4.08 (s, 2H), 3.38 (s, 3H). m/z 428 (M+H⁺).

### Example 44

### Synthesis of 2-(4,6-bis(trifluoromethyl)pyrimidin-2-ylamino)-N-(4-fluorophenyl)-N-methylacetamide

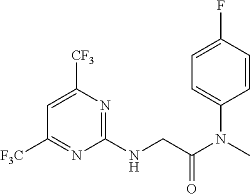 [SMILES: CN(C(=O)CNc1nc(C(F)(F)F)cc(C(F)(F)F)n1)c1ccc(F)cc1]

### Step 1. Preparation of 4,6-bis(trifluoromethyl)pyrimidin-2-ol

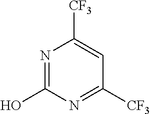 [SMILES: Oc1nc(C(F)(F)F)cc(C(F)(F)F)n1]

To a solution of urea (0.57 g, 9.5 mmol) in EtOH (10 mL) was added 1,1,1,5,5,5-hexafluoropentane-2,4-dione (2.0 g, 9.6 mmol) and concentrated H₂SO₄ (0.05 mL). The mixture was stirred overnight at 80° C. and then diluted with water and extracted with DCM. The combined organic layers were dried over Na₂SO₄ and concentrated under reduced pressure to afford 0.9 g of the title compound as a black solid which was used without further purification.

### Step 2. Preparation of 2-chloro-4,6-bis(trifluoromethyl)pyrimidine

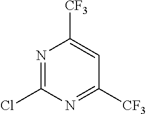 [SMILES: FC(F)(F)c1cc(C(F)(F)F)nc(Cl)n1]

To a solution of 4,6-bis(trifluoromethyl)pyrimidin-2-ol (1.0 g, 4.3 mmol) in POCl₃ (5 mL) was added Et₃N (0.44 g, 4.3 mmol) dropwise. The mixture was stirred overnight at 120° C. Ice water (50 mL) was added slowly and the mixture was extracted with DCM and the combined organic layers were concentrated under reduced pressure to afford 200 mg of the title compound as black oil which was used without further purification.

### Step 3. Preparation of 2-(4,6-bis(trifluoromethyl)pyrimidin-2-ylamino)-N-(4-fluorophenyl)-N-methylacetamide

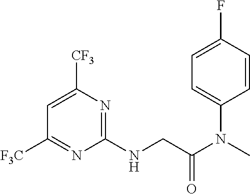 [SMILES: CN(C(=O)CNc1nc(C(F)(F)F)cc(C(F)(F)F)n1)c1ccc(F)cc1]

The title compound was prepared using General Procedure B employing 2-chloro-4,6-bis(trifluoromethyl)pyrimidine and 2-amino-N-(4-fluorophenyl)-N-methylacetamide TFA salt (Example 19, Step 2). The mixture was stirred for 4 h at 50° C. under nitrogen and then diluted with water and extracted with EtOAc. The combined organic layer was concentrated under reduced pressure and the residue was purified by Prep-TLC (3:1, PE:EtOAc) to afford 60 mg (38% yield) of the title compound as a white solid. ¹H NMR (300 MHz; DMSO-d₆): δ 3.16 (s, 3H), 3.78-3.85 (m, 2H), 7.29-7.45 (m, 5H), 8.69-8.73 (m, 1H) ppm. m/z 397 (M+H⁺).

### Example 45

### Synthesis of 3-(2-(3-cyano-4,6-bis(trifluoromethyl)pyridin-2-ylamino)-N-methylacetamido)benzamide

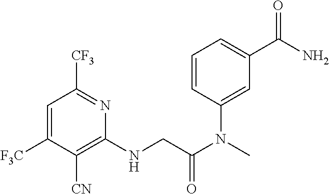 [SMILES: CN(C(=O)CNc1nc(C(F)(F)F)cc(C(F)(F)F)c1C#N)c1cccc(C(N)=O)c1]

### Step 1. Preparation of tert-butyl (2-((3-cyanophenyl)(methyl)amino)-2-oxoethyl)carbamate

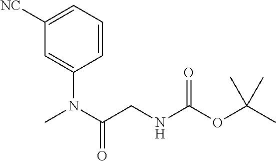 [SMILES: CN(C(=O)CNC(=O)OC(C)(C)C)c1cccc(C#N)c1]

To a solution of 3-(methylamino)benzonitrile (500 mg, 3.9 mmol) in THE (5 mL) was added [(tert-butoxycarbonyl)amino]acetic acid, DIEA (978 mg, 7.6 mmol) and propylphosphonic anhydride solution (3610 mg, 5.7 mmol, 50 wt % in EtOAc) under nitrogen. The mixture was stirred for 4 h at 50° C. under nitrogen and then diluted with water and extracted with EtOAc. The combined organic layers were concentrated under reduced pressure and the residue was purified by Prep-TLC with (1:2 EtOAc:PE) to afford 510 mg (47% yield) of the title compound as a white solid.

### Step 2. Preparation of tert-butyl 2-((3-carbamoylphenyl)(methyl)amino)-2-oxoethylcarbamate

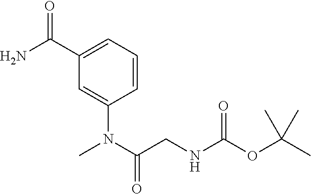 [SMILES: CN(C(=O)CNC(=O)OC(C)(C)C)c1cccc(C(N)=O)c1]

To a solution of tert-butyl 2-((3-cyanophenyl)(methyl)amino)-2-oxoethylcarbamate (500 mg, 1.7 mmol) in MeOH (25 mL) and water (5 mL) was added NaOH (83 mg, 2.1 mmol) and H₂O₂ (2.50 mL). The mixture was stirred for 2 h at room temperature under nitrogen and then diluted with water and extracted with EtOAc. The combined organic layers were concentrated under reduced pressure and the residue was purified using silica gel chromatography (eluent: 9% MeOH in DCM) to afford 300 mg (56% yield) of the title compound as a white solid.

### Step 3. Preparation of 3-(2-amino-N-methylacetamido)benzamide TFA salt

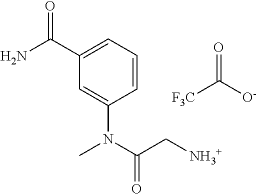

To a solution of tert-butyl 2-((3-carbamoylphenyl)(methyl)amino)-2-oxoethylcarbamate (300 mg, 0.98 mmol) in DCM (3.0 mL) was added TFA (1.0 mL). The mixture was stirred for 2 h at room temperature and then concentrated under reduced pressure. The crude product was purified by Flash-Prep-HPLC (C₁₈ silica gel column; eluent: 15-60% MeCN in water) to afford 170 mg (84% yield) of the title compound as a white solid.

### Step 4. Preparation of 3-(2-(3-cyano-4,6-bis(trifluoromethyl)pyridin-2-ylamino)-N-methylacetamido)benzamide

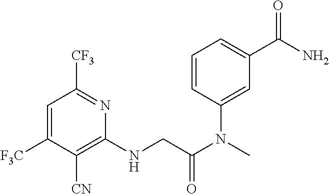 [SMILES: CN(C(=O)CNc1nc(C(F)(F)F)cc(C(F)(F)F)c1C#N)c1cccc(C(N)=O)c1]

The title compound was prepared using General Procedure B employing 3-(2-amino-N-methylacetamido)benzamide TFA salt and 2-chloro-4,6-bis(trifluoromethyl) pyridine-3-carbonitrile (Intermediate A). The mixture was stirred for 4 h at 50° C. under nitrogen. The mixture was diluted with water and extracted with EtOAc. The combined organic layers were concentrated under reduced pressure and the residue was purified by Prep-TLC (50:1, DCM:MeOH) to afford 60 mg (35%) of the title compound as a white solid. ¹H NMR (400 MHz; DMSO-d₆): δ 3.22 (s, 3H), 3.90-3.92 (m, 2H), 7.38 (s, 1H), 7.40-7.57 (m, 3H), 7.78-7.86 (m, 2H), 8.06 (s, 1H), 8.33 (t, 1H) ppm. m/z 446 (M+H⁺).

### Example 46

### Synthesis of N-(benzofuran-5-yl)-2-((3-cyano-4,6-bis(trifluoromethyl)pyridin-2-yl)amino)-N-methylacetamide

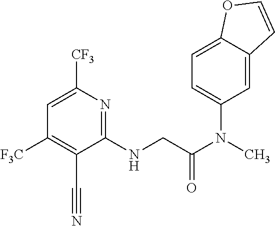 [SMILES: CN(C(=O)CNc1nc(C(F)(F)F)cc(C(F)(F)F)c1C#N)c1ccc2occc2c1]

### Step 1: Preparation of tert-butyl benzofuran-5-ylcarbamate

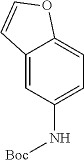 [SMILES: CC(C)(C)OC(=O)Nc1ccc2occc2c1]

To a stirred solution of 1-benzofuran-5-amine (500 mg, 3.8 mmol) and (Boc)₂O (983 mg, 4.5 mmol) in THF (10 mL) was added DIEA (970 mg, 7.5 mmol) dropwise at room temperature. The mixture was stirred overnight at room temperature and then diluted with water. The aqueous layer was extracted with EtOAc and the organic phase was concentrated under vacuum. The residue was purified by silica gel column chromatography (eluent: 3% EtOAc in PE) to afford the title compound (642 mg, 73% yield) as a yellow oil.

### Step 2: Preparation of tert-butyl benzofuran-5-yl(methyl)carbamate

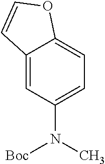 [SMILES: CN(C(=O)OC(C)(C)C)c1ccc2occc2c1]

To a stirred solution tert-butyl benzofuran-5-ylcarbamate (642 mg, 2.8 mmol) in DMF (5 mL) was added NaH (132 mg, 3.3 mmol, as a 60% dispersion in mineral oil) portion wise at 0° C. under nitrogen atmosphere. The mixture was stirred for 0.5 h at 0° C. and then Mel (468 mg, 3.3 mmol) was added at 0° C. The mixture was stirred for an additional 3 h at room temperature and then quenched with sat. aq. NH₄Cl. The mixture was extracted with EtOAc and the organic extracts were combined and concentrated under vacuum. The residue was purified via silica gel chromatography (eluent: 3% EtOAc in PE) to afford the title compound (610 mg, 89% yield) as a yellow oil.

### Step 3: Preparation of N-methylbenzofuran-5-amine

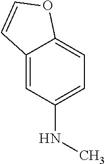

To a round-bottom flask was added tert-butyl benzofuran-5-yl(methyl)carbamate (610 mg), DCM (5 mL) and TFA (1 mL) at room temperature. The mixture was stirred for 2 h at room temperature and then concentrated under vacuum. The residue was purified by preparatory TLC (eluent: 33% EtOAc in PE) to afford the title compound (406 mg) as a yellow oil.

### Step 4: Preparation of tert-butyl (2-(benzofuran-5-yl(methyl)amino)-2-oxoethyl)carbamate

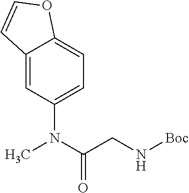 [SMILES: CN(C(=O)CNC(=O)OC(C)(C)C)c1ccc2occc2c1]

To a vial was added N-methylbenzofuran-5-amine (406 mg, 2.6 mmol), [(tert-butoxycarbonyl)amino]acetic acid (531 mg, 3.0 mmol), THE (5 mL), DIEA (713 mg, 5.5 mmol), propylphosphonic anhydride (2.6 g, 4.1 mmol, 50% by weight solution in EtOAc). The mixture was stirred overnight at room temperature and then diluted with water and the aqueous phase was extracted with EtOAc. The combined organic extracts were concentrated under vacuum and the residue was purified by preparatory-TLC (eluent: 33% EtOAc in PE) to afford the title compound (385 mg, 45% yield) as a white solid.

### Step 5: Preparation of 2-amino-N-(benzofuran-5-yl)-N-methylacetamide TFA salt

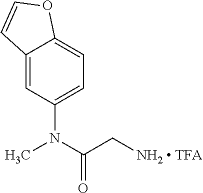

To a vial was added tert-butyl (2-(benzofuran-5-yl(methyl)amino)-2-oxoethyl)carbamate (385 mg, 1.3 mmol), DCM (3 mL), and TFA (0.60 mL). The solution was stirred for 4 h at room temperature and then concentrated under vacuum to afford 300 mg of the title compound as a yellow oil which was used in the next step without further purification.

### Step 6: Preparation of N-(benzofuran-5-yl)-2-((3-cyano-4,6-bis(trifluoromethyl)pyridin-2-yl)amino)-N-methylacetamide

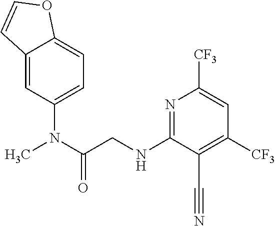 [SMILES: CN(C(=O)CNc1nc(C(F)(F)F)cc(C(F)(F)F)c1C#N)c1ccc2occc2c1]

The title compound was prepared according to General Procedure B utilizing 2-amino-N-(benzofuran-5-yl)-N-methylacetamide TFA salt (150 mg, 0.734 mmol) and Intermediate A (201 mg, 0.734 mmol). The mixture was diluted with EtOAc and washed with a 1M LiCl aq. solution. The organic phase was concentrated under vacuum and the residue was purified by preparatory-TLC (eluent: 33% EtOAc in PE) to afford the title compound (151 mg, 46% yield) as a light yellow solid. ¹H NMR (400 MHz; DMSO-d₆): δ 8.28 (s, 1H), 8.09 (s, 1H), 7.73-7.68 (m, 2H), 7.42 (s, 1H), 7.32 (d, 1H), 7.00 (s, 1H), 3.86 (s, 2H), 3.21 (s, 3H); m z 443 (M+H⁺)

### Example 47

### Synthesis of 2-((3-cyano-4,6-bis(trifluoromethyl)pyridin-2-yl)amino)-N-(4-fluorophenyl)-N-(methyl-d₃)acetamide

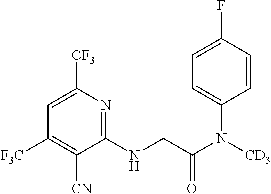 [SMILES: [2H]C([2H])([2H])N(C(=O)CNc1nc(C(F)(F)F)cc(C(F)(F)F)c1C#N)c1ccc(F)cc1]

### Step 1: Preparation of tert-butyl (4-fluorophenyl)carbamate

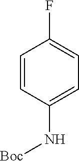 [SMILES: CC(C)(C)OC(=O)Nc1ccc(F)cc1]

To a round-bottom flask was added 4-fluoroaniline (5.0 g, 45 mmol), DCM (50 mL), Et₃N (13.6 g, 0.135 mmol), (Boc)₂O (11.7 g, 0.054 mmol), and DMAP (0.55 g, 0.004 mmol). The solution was stirred overnight at room temperature and the mixture was quenched with water at room temperature. The aqueous layer was extracted with DCM and the combined organic extracts were concentrated under vacuum. The residue was purified by silica gel column chromatography (eluent: 1% EtOAc in PE) to afford the title compound (7.8 g, 82% yield) as a white solid.

### Step 2: Preparation of tert-butyl (4-fluorophenyl)(methyl-d₃)carbamate

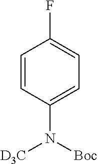 [SMILES: [2H]C([2H])([2H])N(C(=O)OC(C)(C)C)c1ccc(F)cc1]

To a round-bottom flask under an inert atmosphere of nitrogen was added tert-butyl (4-fluorophenyl)carbamate (5.0 g, 23.6 mmol), and DMF (50 mL). The mixture was cooled to 0° C. and NaH (1.13 g, 28.3 mmol as a 60% dispersion in mineral oil) was added. The solution was stirred for 1 h at 0° C., then CD₃I (6.86 g, 47.3 mmol) was added at 0° C. The mixture was stirred overnight at 0° C. and then quenched with sat. aq. NH₄Cl at room temperature. The aqueous layer was extracted with EtOAc and the combined organic extracts were concentrated under vacuum. The residue was purified by silica gel column chromatography (eluent: 3% EtOAc in PE) to afford the title compound (4.5 g, 83% yield) as an off-white solid.

### Step 3: Preparation of 4-fluoro-N-(methyl-d₃)aniline

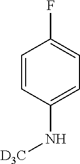 [SMILES: [2H]C([2H])([2H])Nc1ccc(F)cc1]

To a round-bottom flask was added tert-butyl (4-fluorophenyl)(methyl-d₃)carbamate (4.50 g, 19.7 mmol), HCl in 1,4-dioxane (4 M, 1.44 g, 0.039 mmol), and DCM (40 mL) and the solution was stirred overnight at room temperature. The mixture was concentrated under vacuum and the residue was purified by silica gel column chromatography (eluent: 5% MeOH in DCM) to afford the title compound (2 g, 61% yield) as an off-white solid.

### Step 4: Preparation of tert-butyl (2-((4-fluorophenyl)(methyl-d₃)amino)-2-oxoethyl)carbamate

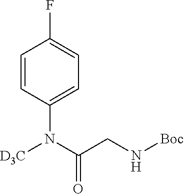 [SMILES: [2H]C([2H])([2H])N(C(=O)CNC(=O)OC(C)(C)C)c1ccc(F)cc1]

To a round-bottom flask was added 4-fluoro-N-(methyl-d₃)aniline (1.54 g, 12.0 mmol), (tert-butoxycarbonyl)glycine (3.16 g, 18.0 mmol), THF (20 mL), DIEA (4.66 g, 3.05 mmol), propylphosphonic anhydride solution (50% by wt in EtOAc, 11.48 g, 18.03 mmol). The solution was stirred overnight at 50° C. and then quenched with water at room temperature. The aqueous layer was extracted with EtOAc and the combined organic extracts were concentrated under vacuum. The residue was purified by silica gel column chromatography (eluent: 9% EtOAc in PE) to afford the title compound (3.2 g, 93% yield) as a light brown solid.

### Step 5: Preparation of 2-amino-N-(4-fluorophenyl)-N-(methyl-d₃)acetamide

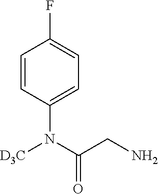 [SMILES: [2H]C([2H])([2H])N(C(=O)CN)c1ccc(F)cc1]

To a round-bottom flask was added tert-butyl (2-((4-fluorophenyl)(methyl-d₃)amino)-2-oxoethyl)carbamate (3.18 g, 11.1 mmol), DCM (30 mL), HCl in 1,4-dioxane (4 M, 5.57 mL 22.2 mmol). The solution was stirred overnight at room temperature and the mixture was basified to pH 8 with sat. aq. NaHCO₃. The aqueous layer was extracted with EtOAc and the combined organic extracts were concentrated under vacuum. The residue was purified by silica gel column chromatography (eluent: 33% EtOAc in PE) to afford the title compound (1.2 g, 58% yield) as a light brown solid.

### Step 6: Preparation of 2-((3-cyano-4,6-bis(trifluoromethyl)pyridin-2-yl)amino)-N-(4-fluorophenyl)-N-(methyl-d₃)acetamide

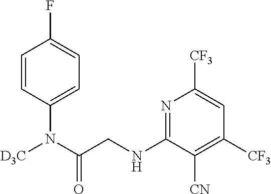 [SMILES: [2H]C([2H])([2H])N(C(=O)CNc1nc(C(F)(F)F)cc(C(F)(F)F)c1C#N)c1ccc(F)cc1]

The title compound was prepared according to General Procedure B employing 2-amino-N-(4-fluorophenyl)-N-(methyl-d₃)acetamide (200 mg, 1.08 mmol) and Intermediate A (296 mg, 1.08 mmol). The mixture was cooled to room temperature and quenched with water at room temperature. The aqueous layer was extracted with EtOAc and the organic layer was washed with an aq. 1 M LiCl solution. The organic phase was concentrated under vacuum and the residue was purified by preparatory-TLC (17% EtOAc in PE) to afford the title compound (100 mg, 22% yield) as a light yellow solid. ¹H NMR (300 MHz; DMSO-d₆): δ 8.37 (s, 1H), 7.74-7.33 (m, 5H), 3.85 (s, 2H); m z 424 (M+H⁺)

### Example 48

### Synthesis of N-(2-chloro-4-fluorophenyl)-2-((3-cyano-4,6-bis(trifluoromethyl)pyridin-2-yl)-amino)-N-methylacetamide

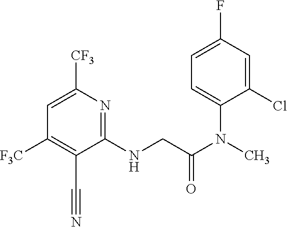 [SMILES: CN(C(=O)CNc1nc(C(F)(F)F)cc(C(F)(F)F)c1C#N)c1ccc(F)cc1Cl]

### Step 1: Preparation of 2-chloro-N-(2-chloro-4-fluorophenyl)-N-methylacetamide

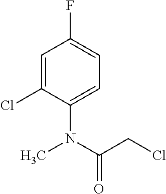

To a solution of 2-chloro-4-fluoro-N-methylaniline (140 mg, 0.877 mmol) and Et₃N (177 mg, 1.75 mmol) in DCM (5 mL) was added a solution of chloroacetyl chloride (198 mg, 1.75 mmol) in DCM (2 mL) dropwise at 0° C. The mixture was warmed to room temperature and stirred for 5 h. The mixture was diluted with EtOAc, washed with sat. aq. NaCl, and concentrated under vacuum. The residue was purified by preparatory-TLC (eluent: 25% EtOAc in PE) to afford the title compound (145 mg, 70% yield) as a colorless oil.

### Step 2: Preparation of N-(2-chloro-4-fluorophenyl)-2-(1,3-dioxoisoindolin-2-yl)-N-methylacetamide

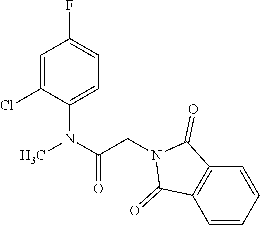 [SMILES: CN(C(=O)CN1C(=O)c2ccccc2C1=O)c1ccc(F)cc1Cl]

To a solution of 2-chloro-N-(2-chloro-4-fluorophenyl)-N-methylacetamide (145 mg, 0.614 mmol) in DMF (3 mL) was added 2-potassioisoindole-1,3-dione (227 mg, 1.22 mmol) portion wise at room temperature. The mixture was stirred at room temperature for 45 h and then poured into H₂O and extracted with EtOAc. The combined organic extracts were concentrated under vacuum and the residue was purified by preparatory-TLC (eluent: 25% EtOAc in PE) to afford the title compound (160 mg, 75% yield) as a white solid.

### Step 3: Preparation of 2-amino-N-(2-chloro-4-fluorophenyl)-N-methylacetamide

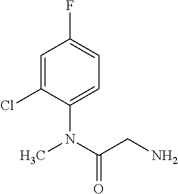

To a solution of N-(2-chloro-4-fluorophenyl)-2-(1,3-dioxoisoindolin-2-yl)-N-methylacetamide (160 mg, 0.461 mmol) in EtOH (5 mL) was added a solution of hydrazine hydrate (98%) (133 mg, 2.65 mmol) in EtOH (2 mL) dropwise at room temperature. The mixture was warmed to 50° C. and stirred for 2 h. The mixture was cooled to room temperature and filtered. The filter cake was washed with EtOH and the filtrate was concentrated under vacuum. The residue was purified by preparatory-HPLC (C18 Spherical Column, 20-35 um, 40 g; gradient elution: 0%-60% MeCN in H₂O (containing 10 mmol/L NH₄HCO₃); run time 30 min; flow rate: 25 ml/min; UV detection at 254 nm) to afford the title compound (86 mg, 86% yield) as colorless oil.

### Step 4: N-(2-chloro-4-fluorophenyl)-2-((3-cyano-4,6-bis(trifluoromethyl)pyridin-2-yl)amino)-N-methylacetamide

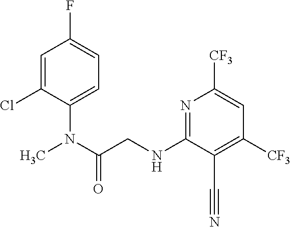 [SMILES: CN(C(=O)CNc1nc(C(F)(F)F)cc(C(F)(F)F)c1C#N)c1ccc(F)cc1Cl]

The title compound was prepared according to General Procedure B employing 2-amino-N-(2-chloro-4-fluorophenyl)-N-methylacetamide (76 mg, 0.351 mmol) and Intermediate B (240 mg, 0.526 mmol). The mixture was heated to 50° C. for 4 h, then cooled to room temperature, diluted with EtOAc and washed with 1M LiCl aq. solution The organic phase was concentrated under vacuum and the residue was purified by preparatory-TLC (eluent: 25% EtOAc in hexanes) to afford the title compound (35 mg, 22% yield) as a pink solid. ¹H NMR (300 MHz; DMSO-d₆): δ 8.35-8.26 (m, 1H), 7.74-7.70 (m, 1H), 7.62-7.57 (m, 1H), 7.48-7.31 (m, 2H), 3.88-3.73 (m, 2H), 3.09 (s, 3H); m z 330 (M+H⁺).

### Example 49

### Synthesis of 2-((3-cyano-4,6-bis(trifluoromethyl)pyridin-2-yl)amino)-N-(2-cyano-4-fluorophenyl)-N-methylacetamide

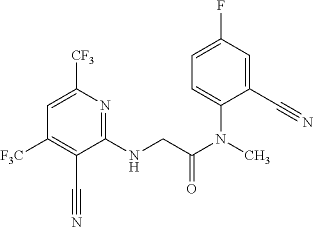 [SMILES: CN(C(=O)CNc1nc(C(F)(F)F)cc(C(F)(F)F)c1C#N)c1ccc(F)cc1C#N]

### Step 1: Preparation of 5-fluoro-2-(methylamino)benzonitrile

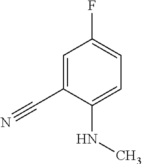

To a solution of DIEA (1.39 g, 10.7 mmol) in methylamine (2M in THF, 12 mL) was added 2,5-difluorobenzonitrile (600 mg, 4.31 mmol) and the mixture was stirred at 80° C. for 17 h. The mixture was cooled to room temperature, diluted with EtOAc, and washed with sat. aq. NaCl. The organic phase was concentrated under vacuum and the residue was purified by silica gel column chromatograph (eluent: 9% EtOAc in PE) to afford the title compound (460 mg, 71% yield) as a white solid.

### Step 2: Preparation of 2-chloro-N-(2-cyano-4-fluorophenyl)-N-methylacetamide

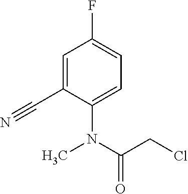

To a solution of 5-fluoro-2-(methylamino)benzonitrile (460 mg, 3.06 mmol) and Et₃N (619 mg, 6.12 mmol) in DCM (16 mL) was added a solution of 2-chloroacetyl chloride (691 mg, 6.12 mmol) in DCM (8 mL) dropwise at 0° C. The mixture was warmed to room temperature and stirred for 12 h, then diluted with DCM and washed with sat. aq. NaCl. The organic phase was concentrated under vacuum and the residue was purified by silica gel column chromatograph (eluent: 14% to 25% EtOAc in PE) to afford the title compound (480 mg, 69% yield) as a light-yellow oil.

### Step 3: Preparation of N-(2-cyano-4-fluorophenyl)-2-(1,3-dioxoisoindolin-2-yl)-N-methyl-acetamide

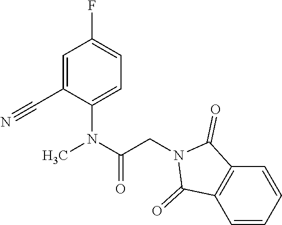 [SMILES: CN(C(=O)CN1C(=O)c2ccccc2C1=O)c1ccc(F)cc1C#N]

To a solution of 2-chloro-N-(2-cyano-4-fluorophenyl)-N-methylacetamide (570 mg, 2.51 mmol) in DMF (9 mL) was added potassium 1,3-dioxoisoindolin-2-ide (931 mg, 5.03 mmol) at room temperature. The mixture was stirred at room temperature for 12 h, then poured into H₂O and the mixture was stirred at room temperature for 15 minutes. The mixture was then filtered and the filter cake was dried under vacuum to afford the title compound (630 mg, 74% yield) as an off-white solid.

### Step 4: Preparation of 2-amino-N-(2-cyano-4-fluorophenyl)-N-methylacetamide

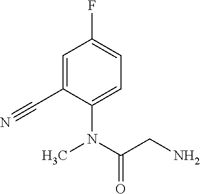

To a solution of N-(2-cyano-4-fluorophenyl)-2-(1,3-dioxoisoindolin-2-yl)-N-methylacetamide (630 mg, 1.86 mmol) in EtOH (50 mL) was added a solution of hydrazine hydrate (98%) (467 mg, 9.3 mmol) in EtOH (2 mL) dropwise at room temperature. The mixture was warmed to 50° C. and stirred for 1.5 h, then cooled to room temperature and filtered. The filter cake was washed with EtOH and the filtrate was concentrated The residue was purified by preparatory-TLC (eluent: 9% MeOH in DCM) to afford the title compound (350 mg, 90% yield) as light yellow oil.

### Step 5: Preparation of 2-((3-cyano-4,6-bis(trifluoromethyl)pyridin-2-yl)amino)-N-(2-cyano-4-fluorophenyl)-N-methylacetamide

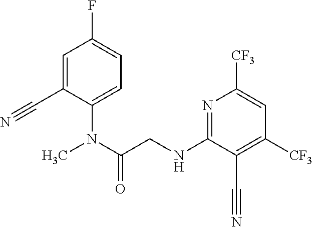 [SMILES: CN(C(=O)CNc1nc(C(F)(F)F)cc(C(F)(F)F)c1C#N)c1ccc(F)cc1C#N]

The title compound was prepared according to General Procedure B using 2-amino-N-(2-cyano-4-fluorophenyl)-N-methylacetamide (120 mg, 0.579 mmol) and Intermediate A (265 mg, 0.579 mmol). The mixture was cooled to room temperature, poured into 1M LiCl aq, and extracted with EtOAc. The combined organic extracts were concentrated under vacuum and the residue was purified by preparatory-TLC (eluent: 33% EtOAc in hexanes) to afford the title compound (35 mg, 13% yield) as an off-white solid. ¹H NMR (300 MHz; DMSO-d₆): δ 8.41-8.32 (m, 1H), 8.07-7.91 (m, 1H), 7.82-7.64 (m, 2H), 7.47-7.43 (m, 1H), 3.96-3.85 (m, 2H), 3.46-3.19 (m, 3H); m/z 446 (M+H⁺).

### Example 50

### Synthesis of 2-((3-cyano-4,6-bis(trifluoromethyl)pyridin-2-yl)amino)-N-methyl-N-(1-methyl-1H-benzo[d]imidazol-5-yl)acetamide

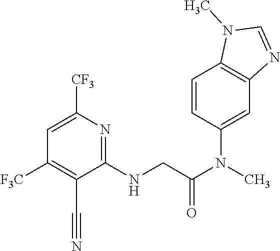 [SMILES: CN(C(=O)CNc1nc(C(F)(F)F)cc(C(F)(F)F)c1C#N)c1ccc2c(c1)ncn2C]

### Step 1: Preparation of N,1-dimethyl-1H-benzo[d]imidazol-5-amine

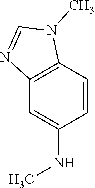

The title compound was prepared according to General Procedure E employing 5-bromo-1-methyl-1H-benzo[d]imidazole (1.00 g, 4.73 mmol), replacing 1,4-dioxane with DMF (8 mL). The mixture was stirred for 6 h at 80° C. and then quenched with H₂O and extracted with DCM. The combined organic extracts were dried over Na₂SO₄ and concentrated under vacuum. The residue was purified by preparatory-TLC (eluent: 3% MeOH in DCM) to afford the title compound (660 mg, 82% yield) as a brown solid.

### Step 2: Preparation of 2-((3-cyano-4,6-bis(trifluoromethyl)pyridin-2-yl)amino)-N-methyl-N-(1-methyl-1H-benzo[d]imidazol-5-yl)acetamide

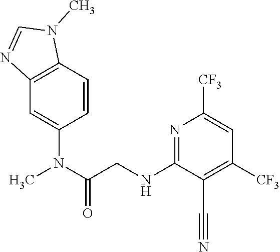 [SMILES: CN(C(=O)CNc1nc(C(F)(F)F)cc(C(F)(F)F)c1C#N)c1ccc2c(c1)ncn2C]

The title compound was prepared according to General Procedure A employing Intermediate B (77 mg, 0.24 mmol) and N,1-dimethyl-1H-benzo[d]imidazol-5-amine (40 mg, 0.24 mmol). The solution was stirred for 2 h at 70° C. and then quenched with H₂O and extracted with DCM. The combined organic extracts were dried over Na₂SO₄ and concentrated under vacuum. The residue was purified by preparatory-TLC (5% MeOH in DCM) to afford the title compound (24 mg, 21% yield) as a yellow solid. ¹H NMR (300 MHz; DMSO-d₆): δ 8.29-8.26 (m, 2H), 7.71-7.68 (m, 2H), 7.43 (s, 1H), 7.29 (d, 1H), 3.88-3.85 (m, 5H), 3.32 (m, 3H); m z 457 (M+H⁺).

### Example 51

### Synthesis of 2-((3-cyano-4,6-bis(trifluoromethyl)pyridin-2-yl)amino)-N-methyl-N-(3-methylbenzofuran-5-yl)acetamide

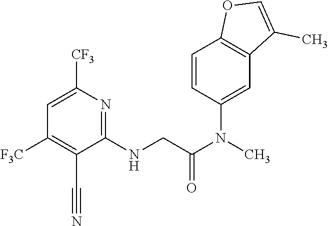 [SMILES: Cc1coc2ccc(N(C)C(=O)CNc3nc(C(F)(F)F)cc(C(F)(F)F)c3C#N)cc12]

### Step 1: Preparation of N,3-dimethylbenzofuran-5-amine

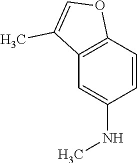

The title compound was prepared according to General Procedure E employing 5-bromo-3-methylbenzofuran (500 mg, 2.36 mmol). The mixture was diluted with EtOAc, washed with H₂O and concentrated under vacuum. The residue was purified by preparatory-TLC (eluent: 33% EtOAc in PE) to afford the title compound (370 mg, 96% yield) as a white solid.

### Step 2: Preparation of 2-((3-cyano-4,6-bis(trifluoromethyl)pyridin-2-yl)amino)-N-methyl-N-(3-methylbenzofuran-5-yl)acetamide

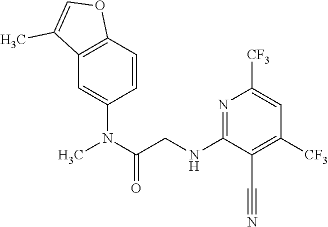 [SMILES: Cc1coc2ccc(N(C)C(=O)CNc3nc(C(F)(F)F)cc(C(F)(F)F)c3C#N)cc12]

The title compound was prepared according to General Procedure A using N,3-dimethylbenzofuran-5-amine (80 mg, 0.49 mmol), and Intermediate B (155 mg, 0.496 mmol). The mixture was heated to 70° C. and stirred for 2 h and then diluted with EtOAc. The mixture was washed with H₂O and the organic phase was concentrated under vacuum. The residue was purified by preparatory-TLC (13% EtOAc in PE) to afford the title compound (124 mg, 55% yield) as an off-white solid. ¹H NMR (300 MHz; DMSO-d₆): δ 8.33 (t, 1H), 8.34-8.30 (m, 1H), 7.65-7.54 (m, 2H), 7.43 (s, 1H), 7.32-7.26 (m, 1H), 3.86 (d, 2H), 3.28 (s, 3H), 2.27 (s, 3H); m z 457 (M+H⁺).

### Example 52

### Synthesis of 2-((3-cyano-4,6-bis(trifluoromethyl)pyridin-2-yl)amino)-N-methyl-N-(2-methylbenzofuran-5-yl)acetamide

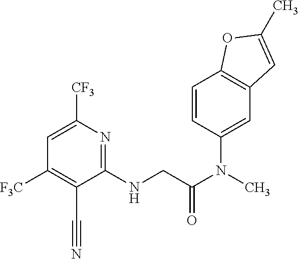 [SMILES: Cc1cc2cc(N(C)C(=O)CNc3nc(C(F)(F)F)cc(C(F)(F)F)c3C#N)ccc2o1]

### Step 1: Preparation of tert-butyl (2-methylbenzofuran-5-yl)carbamate

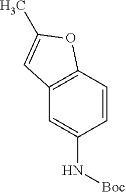 [SMILES: Cc1cc2cc(NC(=O)OC(C)(C)C)ccc2o1]

To a round-bottom flask was added 2-methylbenzofuran-5-amine (500 mg, 3.39 mmol), THF (5 mL), DIEA (878 mg, 6.79 mmol), (Boc)₂O (1.11 g, 5.09 mmol) and the solution was stirred overnight at room temperature. The mixture was diluted with H₂O and the aqueous layer was extracted with EtOAc. The combined organic extracts were concentrated under vacuum and the residue was purified by silica gel column chromatography (eluent: 2% EtOAc in PE) to afford the title compound (650 mg, 77% yield) as a white solid.

### Step 2: Preparation of tert-butyl methyl(2-methylbenzofuran-5-yl)carbamate

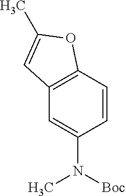 [SMILES: Cc1cc2cc(N(C)C(=O)OC(C)(C)C)ccc2o1]

To a round-bottom flask under an atmosphere of nitrogen was added tert-butyl (2-methylbenzofuran-5-yl)carbamate (640 mg, 2.58 mmol), DMF (6 mL). The solution was cooled to 0° C. and NaH (60% dispersion in mineral oil, 207 mg, 5.17 mmol) was added. The mixture was stirred for 30 minutes at 0° C., then Mel (551 mg, 3.88 mmol) was added at 0° C. The mixture was stirred for 4 hours at 0° C. then warmed to room temperature and quenched with sat. aq. NH₄Cl. The mixture was extracted with EtOAc and the combined organic extracts were washed with 1M LiCl aq. solution and dried over anhydrous Na₂SO₄. The organic phase was filtered and the filtrate was concentrated under vacuum to afford the title compound (650 mg, 96% yield) as a yellow solid.

### Step 3: Preparation of N,2-dimethylbenzofuran-5-amine

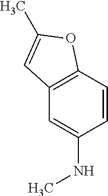

To a round-bottom flask was added tert-butyl methyl(2-methylbenzofuran-5-yl)-carbamate (600 mg, 2.29 mmol), DCM (6 mL) and TFA (1.20 mL) and the solution was stirred for 1 h at room temperature. The mixture was concentrated under vacuum to afford the title compound (400 mg, 98% yield) as a yellow solid.

### Step 4: Preparation of 2-((3-cyano-4,6-bis(trifluoromethyl)pyridin-2-yl)amino)-N-methyl-N-(2-methylbenzofuran-5-yl)acetamide

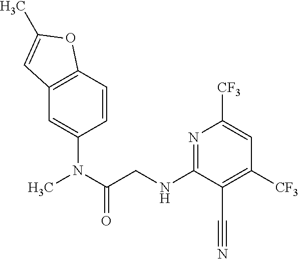 [SMILES: Cc1cc2cc(N(C)C(=O)CNc3nc(C(F)(F)F)cc(C(F)(F)F)c3C#N)ccc2o1]

The title compound was prepared according to General Procedure A using N,2-dimethyl-benzofuran-5-amine (51 mg, 0.31 mmol) and Intermediate B (99 mg, 0.31 mmol). The solution was stirred for 1 h at 70° C. and then diluted with H₂O. The mixture was extracted with DCM and the combined organic extracts were concentrated under vacuum. The residue was purified by preparatory-TLC (50% EtOAc in PE) to afford the title compound (61 mg, 42% yield) as a yellow solid. ¹H NMR (300 MHz; DMSO-d₆): δ 8.29 (t, 1H), 7.71-7.53 (m, 2H), 7.42 (s, 1H), 7.22-7.20 (m, 1H), 6.61 (s, 1H), 3.86 (d, 2H), 3.19 (s, 3H), 2.46 (s, 3H); m z 457 (M+H⁺).

### Example 53

### Synthesis of N-(benzo[b]thiophen-5-yl)-2-((3-cyano-4,6-bis(trifluoromethyl)pyridin-2-yl)-amino)-N-methylacetamide

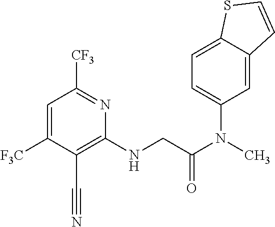 [SMILES: CN(C(=O)CNc1nc(C(F)(F)F)cc(C(F)(F)F)c1C#N)c1ccc2sccc2c1]

### Step 1: Preparation of N-methylbenzo[b]thiophen-5-amine

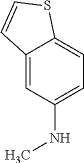

The title compound was prepared according to General Procedure E employing 5-bromobenzo[b]thiophene (1.00 g, 4.69 mmol), and replacing 1,4-dioxane with DMF (10 mL). The solution was stirred for 4 h at 80° C., then cooled to room temperature and diluted with H₂O. The mixture was extracted with EtOAc and the combined organic extracts were concentrated under vacuum. The residue was purified by preparatory-TLC (eluent: 2% MeOH in DCM) to afford the title compound (500 mg, 60% yield) as an off-white oil.

### Step 2: Preparation of N-(benzo[b]thiophen-5-yl)-2-((3-cyano-4,6-bis(trifluoromethyl)pyridin-2-yl)amino)-N-methylacetamide

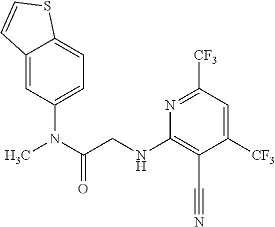 [SMILES: CN(C(=O)CNc1nc(C(F)(F)F)cc(C(F)(F)F)c1C#N)c1ccc2sccc2c1]

The title compound was prepared according to General Procedure A employing N-methylbenzo[b]thiophen-5-amine (60 mg, 0.36 mmol) and Intermediate B (115 mg, 0.368 mmol). The solution was stirred for 1.5 h at 70° C. and then diluted with H₂O. The mixture was extracted with EtOAc and the combined organic extracts were concentrated under vacuum. The residue was purified by preparatory-TLC (eluent: 5% MeOH in DCM) to afford the title compound (97 mg, 57% yield) as a yellow solid. ¹H NMR (300 MHz; DMSO-d₆): δ 8.33 (t, 1H), 8.13 (d, 1H), 7.89 (d, 2H), 7.48-7.35 (m, 3H), 3.90 (s, 2H), 3.23 (s, 3H); m z 459 (M+H⁺).

### Example 54

### Synthesis of 2-((3-cyano-4,6-bis(trifluoromethyl)pyridin-2-yl)amino)-N-methyl-N-(1-methyl-1H-indazol-5-yl)acetamide

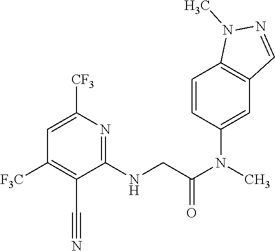 [SMILES: CN(C(=O)CNc1nc(C(F)(F)F)cc(C(F)(F)F)c1C#N)c1ccc2c(cnn2C)c1]

### Step 1: Preparation of N,1-dimethyl-1H-indazol-5-amine

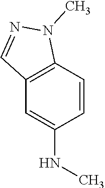

The title compound was prepared according to General Procedure E employing 5-bromo-1-methyl-1H-indazole (950 mg, 4.50 mmol), and replacing 1,4-dioxane with DMF (9.5 mL). The solution was stirred for 4 h at 80° C. and then diluted with H₂O. The mixture was extracted with EtOAc and the combined organic extracts were concentrated under vacuum. The residue was purified by preparatory-TLC (eluent: 5% MeOH in DCM) to afford the title compound (330 mg, 44% yield) as a colorless oil.

### Step 2: Preparation of 2-((3-cyano-4,6-bis(trifluoromethyl)pyridin-2-yl)amino)-N-methyl-N-(1-methyl-1H-indazol-5-yl)acetamide

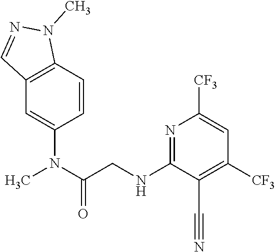 [SMILES: CN(C(=O)CNc1nc(C(F)(F)F)cc(C(F)(F)F)c1C#N)c1ccc2c(cnn2C)c1]

The title compound was prepared according to General Procedure E employing N,1-dimethyl-1H-indazol-5-amine (80 mg, 0.49 mmol) and Intermediate B (155 mg, 0.496 mmol). The solution was stirred for 2 h at 70° C. and then diluted with H₂O. The mixture was extracted with EtOAc and the combined organic extracts were concentrated under vacuum. The residue was purified by preparatory-TLC (eluent: 5% MeOH in DCM) to afford the title compound (84 mg, 35% yield) as a light yellow solid. ¹H NMR (300 MHz; DMSO-d₆): δ 8.30 (t, 1H), 8.08 (s, 1H), 7.77 (d, 2H), 7.41-7.37 (m, 2H), 4.07 (s, 3H), 3.86 (d, 2H), 3.21 (s, 3H); m z 457 (M+H⁺).

### Example 55

### Synthesis of 2-((3-cyano-4,6-bis(trifluoromethyl)pyridin-2-yl)amino)-N-methyl-N-(1-methyl-1H-indol-5-yl)acetamide

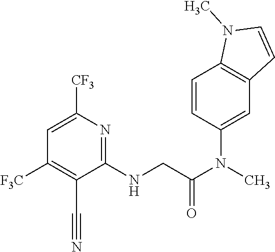 [SMILES: CN(C(=O)CNc1nc(C(F)(F)F)cc(C(F)(F)F)c1C#N)c1ccc2c(ccn2C)c1]

### Step 1: Preparation of N,1-dimethyl-1H-indol-5-amine

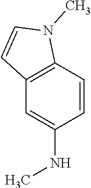

The title compound was prepared according to General Procedure E employing 5-bromo-1-methyl-1H-indole (950 mg, 4.50 mmol) and replacing 1,4-dioxane with DMF (9.5 mL). The solution was stirred for 4 h at 80° C. and then diluted with H₂O (150 mL). The mixture was extracted with EtOAc and the residue was purified by silica gel column chromatography (eluent: 33% EtOAc in PE) to afford the title compound (710 mg, 42% yield) as a light yellow oil.

### Step 2: Preparation of 2-((3-cyano-4,6-bis(trifluoromethyl)pyridin-2-yl)amino)-N-methyl-N-(1-methyl-1H-indol-5-yl)acetamide

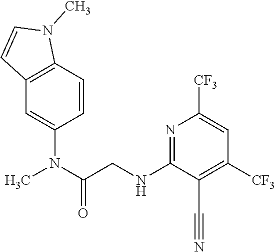 [SMILES: CN(C(=O)CNc1nc(C(F)(F)F)cc(C(F)(F)F)c1C#N)c1ccc2c(ccn2C)c1]

The title compound was prepared according to General Procedure A employing N,1-dimethyl-1H-indol-5-amine (62 mg, 0.387 mmol) and Intermediate B (121 mg, 0.387 mmol). The solution was stirred for 2 h at room temperature and then diluted with H₂O. The mixture was extracted with EtOAc and the combined organic extracts were concentrated under vacuum. The residue was purified by preparatory-TLC (eluent: 25% EtOAc in PE) to afford the title compound (105 mg, 58% yield) as a yellow solid. ¹H NMR (300 MHz; DMSO-d₆): δ 8.24 (t, 1H), 7.56-7.53 (m, 2H), 7.43-7.41 (m, 2H), 7.15-7.11 (m, 1H), 6.46 (d, 1H), 3.87-3.84 (m, 2H), 3.82 (s, 3H), 3.20 (s, 3H); m z 456 (M+H⁺).

### Example 56

### Synthesis of 2-((3-cyano-4,6-bis(trifluoromethyl)pyridin-2-yl)amino)-N-(1H-indol-5-yl)-N-methylacetamide

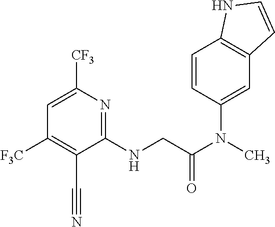 [SMILES: CN(C(=O)CNc1nc(C(F)(F)F)cc(C(F)(F)F)c1C#N)c1ccc2[nH]ccc2c1]

### Step 1: Preparation of tert-butyl 5-bromo-1H-indole-1-carboxylate

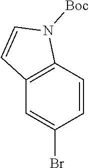 [SMILES: CC(C)(C)OC(=O)n1ccc2cc(Br)ccc21]

To a round-bottom flask was added 5-bromoindole (2.00 g, 10.2 mmol), THE (20 mL), (Boc)₂O (3.34 g, 15.3 mmol), and DIEA (2.64 g, 20.4 mmol). The mixture was stirred at room temperature for 3 h. and then diluted with H₂O. The aqueous layer was extracted with EtOAc and the combined organic extracts were concentrated under vacuum. The residue was purified by silica gel column chromatography (eluent: 9% EtOAc in PE) to afford the title compound (1.6 g, 52% yield) as a yellow solid.

### Step 2: Preparation of tert-butyl 5-(methylamino)-1H-indole-1-carboxylate

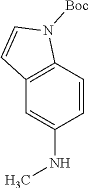 [SMILES: CNc1ccc2c(ccn2C(=O)OC(C)(C)C)c1]

The title compound was prepared according to General Procedure E employing tert-butyl 5-bromo-1H-indole-1-carboxylate (1.58 g, 5.33 mmol). The mixture was stirred at 100° C. overnight and then diluted with H₂O. The aqueous layer was extracted with EtOAc and the combined organic extracts were concentrated under vacuum. The residue was purified by silica gel column chromatography (eluent: 100% DCM) to afford the title compound (1 g, 76% yield) as a yellow solid.

### Step 3: Preparation of tert-butyl 5-(2-((3-cyano-4,6-bis(trifluoromethyl)pyridin-2-yl)amino)-N-methylacetamido)-1H-indole-1-carboxylate

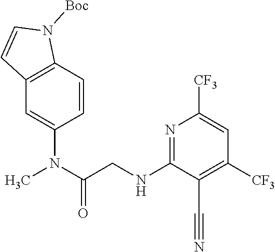 [SMILES: CN(C(=O)CNc1nc(C(F)(F)F)cc(C(F)(F)F)c1C#N)c1ccc2c(ccn2C(=O)OC(C)(C)C)c1]

The title compound was prepared according to General Procedure A employing tert-butyl 5-(methylamino)-1H-indole-1-carboxylate (100 mg, 0.406 mmol) and Intermediate B (119 mg, 0.406 mmol). The mixture was diluted with EtOAc and washed with H₂O. The combined organic extracts were concentrated under vacuum and the residue was purified by preparatory-TLC (eluent: 1% MeOH in DCM) to afford the title compound (200 mg, 90% yield) as a white solid.

### Step 4: Preparation of 2-((3-cyano-4,6-bis(trifluoromethyl)pyridin-2-yl)amino)-N-(1H-indol-5-yl)-N-methylacetamide

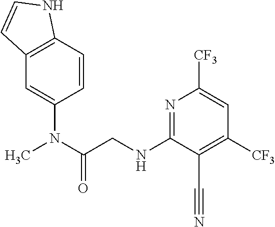 [SMILES: CN(C(=O)CNc1nc(C(F)(F)F)cc(C(F)(F)F)c1C#N)c1ccc2[nH]ccc2c1]

To a round-bottom flask was added tert-butyl 5-(2-((3-cyano-4,6-bis(trifluoromethyl)-pyridin-2-yl)amino)-N-methylacetamido)-1H-indole-1-carboxylate (190 mg, 0.351 mmol), DCM (2 mL), and TFA (0.40 mL) and the mixture was stirred at room temperature for 3 h. The mixture was concentrated under vacuum and the residue was triturated with MeOH (2 mL) to afford the title compound (88 mg, 57% yield) as a white solid. ¹H NMR (300 MHz; DMSO-d₆): δ 11.30 (s, 1H), 8.22 (t, 1H), 7.53-7.40 (m, 4H), 7.08-7.04 (m, 1H), 6.46 (s, 1H), 3.92-3.85 (m, 2H), 3.20 (s, 3H); m z 442 (M+H⁺).

### Example 57

### Synthesis of 2-((3-cyano-4,6-bis(trifluoromethyl)pyridin-2-yl)amino)-N-methyl-N-(quinazolin-6-yl)acetamide

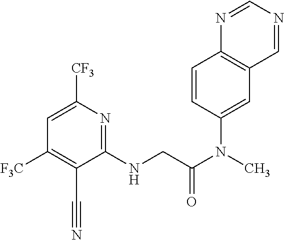 [SMILES: CN(C(=O)CNc1nc(C(F)(F)F)cc(C(F)(F)F)c1C#N)c1ccc2ncncc2c1]

### Step 1: Preparation of N-methylquinazolin-6-amine

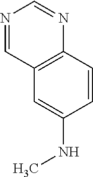

The title compound was prepared according to General Procedure E employing 6-bromoquinazoline (500 mg, 2.39 mmol) and replacing 1,4-dioxane with DMF (5 mL). The solution was stirred for 5 h at 80° C. and then diluted with H₂O. The aqueous layer was extracted with EtOAc and the combined organic extracts were concentrated under vacuum. The residue was purified by preparatory-TLC (eluent: 5% MeOH in DCM) to afford the title compound (270 mg, 69% yield) as a light yellow oil.

### Step 2: Preparation of 2-((3-cyano-4,6-bis(trifluoromethyl)pyridin-2-yl)amino)-N-methyl-N-(quinazolin-6-yl)acetamide

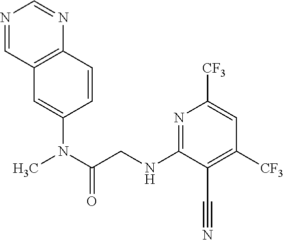 [SMILES: CN(C(=O)CNc1nc(C(F)(F)F)cc(C(F)(F)F)c1C#N)c1ccc2ncncc2c1]

The title compound was prepared according to General Procedure A employing N-methylquinazolin-6-amine (150 mg, 0.942 mmol) and Intermediate B (295 mg, 0.942 mmol), The solution was stirred for 4 h at 70° C. and then diluted with H₂O. The aqueous layer was extracted with EtOAc and the combined organic extracts were concentrated under vacuum. The residue was purified by reverse phase silica gel column chromatography (C18 silica gel; gradient elution: 0% to 60% MeCN in water (containing 10 mmol/L NH₄HCO₃) over 40 minutes; detector UV 254 nm to afford the title compound (44.9 mg, 10% yield) as a white solid. ¹H NMR (300 MHz; DMSO-d₆): δ 9.60 (s, 1H), 9.34 (s, 1H), 8.42 (t, 1H), 8.17 (s, 1H), 8.12-8.09 (m, 1H), 8.03-7.99 (m, 1H), 7.43 (s, 1H), 4.10 (s, 2H), 3.28 (s, 3H); m z 455 (M+H⁺).

### Example 58

### Synthesis of 2-((3-cyano-4,6-bis(trifluoromethyl)pyridin-2-yl)amino)-N-methyl-N-(quinoxalin-6-yl)acetamide

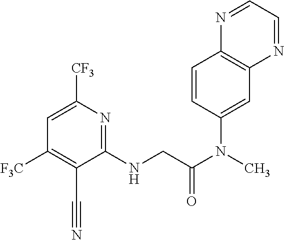 [SMILES: CN(C(=O)CNc1nc(C(F)(F)F)cc(C(F)(F)F)c1C#N)c1ccc2nccnc2c1]

### Step 1: Preparation of N-methylquinoxalin-6-amine

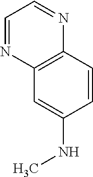

The title compound was prepared according to General Procedure E employing 6-bromoquinoxaline (500 mg, 2.39 mmol) and replacing 1,4-dioxane with DMF (5 mL). The solution was stirred for 5 h at 80° C. and then diluted with H₂O. The aqueous layer was extracted with EtOAc and the combined organic extracts were concentrated under vacuum. The residue was purified by preparatory-TLC (eluent: 5% MeOH in DCM) to afford the title compound (190 mg, 43% yield) as a light-yellow oil.

### Step 2: Preparation of 2-((3-cyano-4,6-bis(trifluoromethyl)pyridin-2-yl)amino)-N-methyl-N-(quinoxalin-6-yl)acetamide

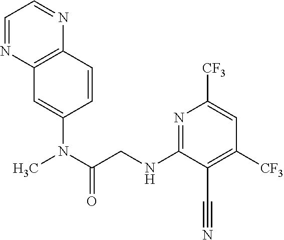 [SMILES: CN(C(=O)CNc1nc(C(F)(F)F)cc(C(F)(F)F)c1C#N)c1ccc2nccnc2c1]

The title compound was prepared according to General Procedure A employing N-methylquinoxalin-6-amine (50 mg, 0.31 mmol), and Intermediate B (98 mg, 0.314 mmol). The solution was stirred for 4 h at 70° C. and then diluted with H₂O. The aqueous layer was extracted with EtOAc and the combined organic extracts were concentrated under vacuum. The residue was purified by reverse phase silica gel column chromatography (C18 silica gel; gradient elution: 0% to 60% MeCN in water (containing 10 mmol/L NH₄HCO₃) over 40 minutes; detector UV 254 nm to afford the title compound (15.5 mg, 10% yield) as a white solid. ¹H NMR (300 MHz; DMSO-d₆): δ 9.00 (d, 2H), 8.19 (d, 1H), 8.11 (s, 1H), 8.11 (s, 1H), 7.89-7.86 (m, 1H), 7.44 (s, 1H), 4.12 (s, 2H), 3.37 (s, 3H); m z 455 (M+H⁺).

### Example 59

### Synthesis of N-(2-acetylisoindolin-5-yl)-2-((3-cyano-4,6-bis(trifluoromethyl)pyridin-2-yl)amino)-N-methylacetamide

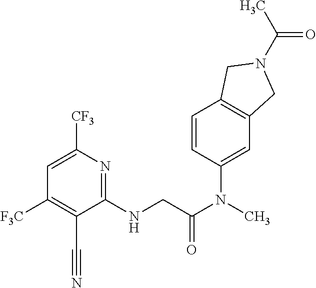 [SMILES: CC(=O)N1Cc2ccc(N(C)C(=O)CNc3nc(C(F)(F)F)cc(C(F)(F)F)c3C#N)cc2C1]

### Step 1: Preparation of 1-(5-bromoisoindolin-2-yl)ethan-1-one

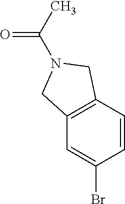

To a round-bottom flask was added 5-bromo-2,3-dihydro-1H-isoindole (2 g, 10 mmol), THF (20 mL), Ac₂O (2.06 g, 20.1 mmol) and Et₃N (2.04 g, 20.1 mmol). The mixture was stirred for 2 h at room temperature and then quenched with H₂O. The aqueous layer was extracted with EtOAc and the combined organic extracts were concentrated under vacuum. The residue was purified by silica gel column chromatography (eluent: 1% EtOAc in PE) to afford the title compound (2.1 g, 86% yield) as a yellow oil.

### Step 2: Preparation of 1-(5-(methylamino)isoindolin-2-yl)ethan-1-one

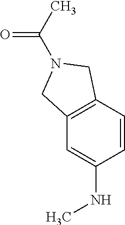

The title compound was prepared according to General Procedure E employing 1-(5-bromoisoindolin-2-yl)ethan-1-one (500 mg, 2.08 mmol). The mixture was stirred for 2 h at 100° C. under nitrogen atmosphere and then diluted with EtOAc. The mixture was washed with H₂O and the organic phase was concentrated under vacuum. The residue was purified by preparatory-TLC (eluent: 9% EtOAc in PE) to afford the title compound (210 mg, 53% yield) as a yellow oil.

### Step 3: Preparation of N-(2-acetylisoindolin-5-yl)-2-((3-cyano-4,6-bis(trifluoromethyl)pyridin-2-yl)amino)-N-methylacetamide

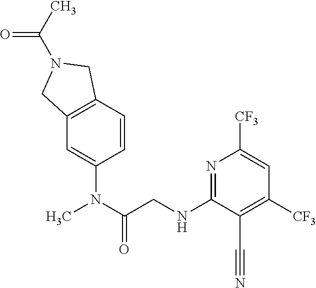 [SMILES: CC(=O)N1Cc2ccc(N(C)C(=O)CNc3nc(C(F)(F)F)cc(C(F)(F)F)c3C#N)cc2C1]

The title compound was prepared according to General Procedure A employing 1-(5-(methylamino)isoindolin-2-yl)ethan-1-one (61 mg, 0.321 mmol) and Intermediate B (100 mg, 0.321 mmol). The mixture was stirred for 1 h at room temperature and then diluted with EtOAc. The mixture was washed with H₂O and the organic phase was concentrated under vacuum. The residue was re-crystallized from MeOH/water (10:1, 2 mL) to afford the title compound (102 mg, 65% yield) as an off-white solid. ¹H NMR (300 MHz; DMSO-d₆): δ 8.34 (s, 1H), 7.53-7.42 (m, 2H), 7.37-7.29 (m, 2H), 4.84 (s, 2H), 4.62 (s, 2H), 3.90 (s, 2H), 3.18 (s, 3H), 2.06 (s, 3H); m z 486 (M+H⁺).

### Example 60

### Synthesis of 2-((3-cyano-4,6-bis(trifluoromethyl)pyridin-2-yl)amino)-N-(3,5-dichlorophenyl)-N-methylacetamide

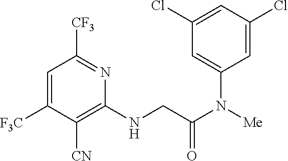 [SMILES: CN(C(=O)CNc1nc(C(F)(F)F)cc(C(F)(F)F)c1C#N)c1cc(Cl)cc(Cl)c1]

### Step 1: Preparation of tert-butyl (2-((3,5-dichlorophenyl)(methyl)amino)-2-oxoethyl)carbamate

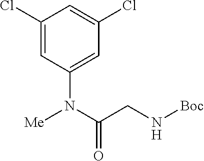 [SMILES: CN(C(=O)CNC(=O)OC(C)(C)C)c1cc(Cl)cc(Cl)c1]

A reaction vessel was charged with 3,5-dichloro-N-methylaniline (125 mg, 0.710 mmol), (tert-butoxycarbonyl)glycine (136 mg, 0.781 mmol), DIEA (183 mg, 1.42 mmol), HATU (323 mg, 0.852 mmol) and THE (1 mL). The mixture was stirred at room temperature overnight and then diluted with H₂O. The aqueous layer was extracted with EtOAc and the combined organic extracts were concentrated under vacuum. The residue was purified by silica gel column chromatography (gradient elution: 0-25% EtOAc in hexanes) to afford the title compound (139 mg, 59% yield).

### Step 2: Preparation of 2-amino-N-(3,5-dichlorophenyl)-N-methylacetamide HCl salt

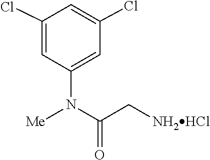

A reaction vessel was charged with tert-butyl (2-((3,5-dichlorophenyl)(methyl)amino)-2-oxoethyl)carbamate (139 mg, 0.417 mmol), HCl in 1,4-dioxane (4M, 0.5 mL) and EtOAc (0.5 mL). The mixture was stirred at room temperature for 6 h and then concentrated under vacuum. The residue was used in the next step without further purification.

### Step 3: Preparation of 2-((3-cyano-4,6-bis(trifluoromethyl)pyridin-2-yl)amino)-N-(3,5-dichlorophenyl)-N-methylacetamide

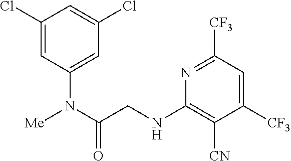 [SMILES: CN(C(=O)CNc1nc(C(F)(F)F)cc(C(F)(F)F)c1C#N)c1cc(Cl)cc(Cl)c1]

The title compound was prepared according to General Procedure B employing 2-amino-N-(3,5-dichlorophenyl)-N-methylacetamide (50 mg, 0.21 mmol) and Intermediate A (64 mg, 0.23 mmol). The mixture was diluted with EtOAc and washed with a 1M LiCl aq. solution. The organic phase was concentrated under vacuum and the residue was purified by silica gel column chromatography (gradient elution: 0-25% EtOAc in hexanes) to afford the title compound (10 mg, 10% yield). ¹H NMR (400 MHz, DMSO-d₆) δ 9.35 (s, 1H), 9.21 (d, 1H), 8.52 (s, 1H), 7.70 (d, 1H), 7.49 (s, 1H), 4.35 (d, 3H), 3.41 (s, 3H); m z 471.00 (M+H⁺)

### Example 61

### Synthesis of N-(4-bromophenyl)-2-((3-cyano-4,6-bis(trifluoromethyl)pyridin-2-yl)amino)-N-methylacetamide

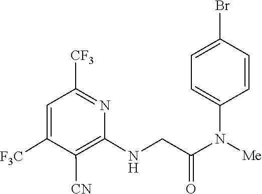 [SMILES: CN(C(=O)CNc1nc(C(F)(F)F)cc(C(F)(F)F)c1C#N)c1ccc(Br)cc1]

The title compound was prepared according to General Procedure A, employing 4-bromo-N-methylaniline (85 mg, 0.46 mmol) and Intermediate B (120 mg, 0.383 mmol). The mixture was diluted with EtOAc and washed with H₂O. The combined organic extracts were concentrated under vacuum and the residue was purified using silica gel chromatography (gradient elution: 0-25% EtOAc in hexanes) to afford the title compound (60 mg, 32% yield). ¹H NMR (400 MHz, DMSO-d₆) δ 8.49-8.24 (m, 1H), 7.79-7.56 (m, 2H), 7.45 (s, 1H), 7.41-7.27 (m, 2H), 3.90 (s, 2H), 3.18 (s, 3H); m z 481.00 (M+H⁺)

### BIOLOGICAL EXAMPLES

### Example 1

The ability of the compounds of Formula (I) to inhibit polymerase activity of Pol theta was determined using the assay described below.

A mixture of 20 uL of Pol theta polymerase domain (residues 1819-2590) at a final concentration of 4 nM in assay buffer (20 m M TRIS, pH 7.80, 50 mM KCl, 10 mM MgCl₂, 1 mM DTT, 0.01% BSA, 0.01% Tween20) was added to test compounds (11-point dilution series of test compounds) except the low control wells without test compounds. The above enzyme and test compound inhibitor mixture was then incubated at room temperature for 15 min. An equal volume (20 μl) of dNTP substrate mixture (48 μM) and primed molecular beacon DNA (obtained by annealing template SEQ ID NO 2: (5′-CCTTCCTCCCGTGTCTTG-TACCTTCCCGTCA-GGAGGAAGG-3′) with 5′-TAMRA and 3′-BHQ and primer DNA (SEQ ID NO: 3; 5′-GACGGGAAGG-3′) in 10 mM Tris-HCl pH 8.0, 100 mM NaCl buffer) (96 nM) in assay buffer was added to all the test wells. The inhibition activity was measured by monitoring the fluorescence change over 30 min at 535 nm upon excitation at 485 nm. The high control (DMSO with enzyme) with high fluorescence intensity represents no inhibition of polymerase reaction while the low control (DMSO with buffer) with low fluorescence intensity represents full inhibition of polymerase activity. Slope of the reaction progress curves were used to calculate the rate of polymerization. The rates were used to determine the percent inhibition using a four-parameter inhibition model to generate IC₅₀, Hill slope and max inhibition.

The IC₅₀ of the compounds in Table 1 above are disclosed in Table 2 below:

| Cpd. No. | Primer extension Assay IC₅₀ |
|---|---|
| 1 | ++ |
| 2 | ++++ |
| 3 | +++ |
| 4 | ++++ |
| 5 | ++++ |
| 6 | ++++ |
| 7 | ++++ |
| 8 | ++++ |
| 9 | ++++ |
| 10 | ++++ |
| 11 | ++++ |
| 12 | ++++ |
| 13 | ++++ |
| 14 | +++ |
| 15 | +++ |
| 16 | +++ |
| 17 | +++ |
| 18 | +++ |
| 19 | ++++ |
| 20 | +++ |
| 21 | +++ |
| 22 | ++++ |
| 23 | ++++ |
| 24 | ++ |
| 25 | ++++ |
| 26 | ++++ |
| 27 | ++++ |
| 28 | ++++ |
| 29 | ++ |
| 30 | +++ |
| 31 | ++ |
| 32 | ++++ |
| 33 | ++++ |
| 34 | ++ |
| 35 | ++++ |
| 36 | +++ |
| 37 | +++ |
| 38 | +++ |
| 39 | ++++ |
| 40 | ++++ |
| 41 | ++++ |
| 42 | ++++ |
| 43 | ++++ |
| 44 | ++++ |
| 45 | ++++ |
| 46 | ++++ |
| 47 | ++++ |
| 48 | ++++ |
| 49 | ++ |
| 50 | ++++ |
| 51 | ++++ |
| 52 | ++++ |
| 53 | ++++ |
| 54 | ++++ |
| 55 | ++++ |
| 56 | ++++ |
| 57 | +++ |
| 58 | ++++ |
| 59 | ++ |
| 60 | ++++ |
| 61 | ++++ |

(+) IC₅₀ = 10 uM-1 uM;
(++) IC₅₀ = 1 uM-500 nM;
(+++) IC₅₀ = 500 nM-200 nM;
(++++) IC₅₀ < 200 nM

What is claimed:

1. A compound of Formula (I): 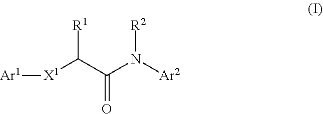
wherein:
X¹ is —NH— or —O—;
Ar¹ is phenyl or six- to ten-membered heteroaryl wherein phenyl and heteroaryl are substituted with R^{a} and further substituted with R^{b} and R^{c}, wherein R^{a} is haloalkyl and R^{b} and R^{c} are independently selected from hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R^{b} and/or R^{c} are unsubstituted or substituted with one, two, or three substituents independently selected from alkyl, halo, haloalkyl, and hydroxy;
R¹ is hydrogen, alkyl, hydroxyalkyl, alkoxyalkyl, aminoalkyl, aminocarbonylalkyl, or phenalkyl wherein phenyl in phenalkyl is substituted with R^{d}, R^{e}, and R, wherein R^{d}, R^{e}, and R^{f} are independently selected from hydrogen, alkyl, halo, haloalkyl, alkoxy, haloalkoxy, hydroxy, and cyano;
R² is alkyl, deuteroalkyl, cycloalkyl, or haloalkyl;
Ar² is phenyl or heteroaryl wherein said phenyl and heteroaryl are substituted with R^{g}, R^{h}, and Rⁱ, wherein R^{g}, R^{h}, and R¹ are independently selected from hydrogen, alkyl, cycloalkyl, cycloalkyloxy, halo, haloalkyl, alkoxy, haloalkoxy, hydroxy, cyano, and —CONH₂; provided one of R^{g}, R^{h}, and Rⁱ is other than hydrogen; or
a pharmaceutically acceptable salt thereof; provided that:
(1) when X¹ is NH, R¹ is hydrogen, R² is methyl or ethyl, and Ar¹ is phenyl substituted with R^{a} and R^{b}, where R^{a} is haloalkyl and R^{b} is hydrogen, chloro, methyl, or piperidin-1-yl, then Ar² is not 3-methylphenyl; and
(2) the compound of Formula (I) is not:
Acetamide, N-(4-fluorophenyl)-N-methyl-2-[[5-(trifluoromethyl)-2-benzothiazolyl]oxy]-;
Acetamide, N-(5-bromo-2-pyridinyl)-N-ethyl-2-[3-(trifluoromethyl)phenoxy]-;
Acetamide, N-ethyl-N-(6-methoxy-3-pyridinyl)-2-[3-(trifluoromethyl)phenoxy]-;
Acetamide, N-ethyl-N-(4-fluorophenyl)-2-[3-(trifluoromethyl)phenoxy]-;
Acetamide, N-ethyl-N-(4-fluorophenyl)-2-[4-(trifluoromethyl)phenoxy]-;
Acetamide, N-(3,4-difluorophenyl)-N-ethyl-2-[4-(trifluoromethyl)phenoxy]-;
Acetamide, N-(3,4-difluorophenyl)-N-ethyl-2-[2-(trifluoromethyl)phenoxy]-;
Acetamide, N-(5-bromo-2-pyridinyl)-N-ethyl-2-[2-(trifluoromethyl)phenoxy]-;
Acetamide, N-(3,4-difluorophenyl)-N-ethyl-2-[3-(trifluoromethyl)phenoxy]-;
Acetamide, N-(4-bromo-2-methylphenyl)-N-methyl-2-[3-(trifluoromethyl)phenoxy]-;
Acetamide, N-(3-fluoro-4-methoxyphenyl)-N-(1-methylethyl)-2-[2-(trifluoromethyl)phenoxy]-;
Benzamide, 4-[methyl[2-[2-(trifluoromethyl)phenoxy]acetyl]amino]-;
Propanamide, 2-[2-chloro-4-(trifluoromethyl)phenoxy]-N-(4-fluorophenyl)-N-(1-methylethyl)-;
Acetamide, 2-[4-(bromomethyl)phenoxy]-N-(3-chlorophenyl)-N-methyl-;
Acetamide, N-ethyl-N-(4-fluorophenyl)-2-[2-(trifluoromethyl)phenoxy]-;
Acetamide, 2-[3,5-bis(trifluoromethyl)phenoxy]-N-(4-methyl-2-thiazolyl)-N-(2,2,2-trifluoroethyl)-;
Acetamido, 2-[3,5-bis(trifluoromethyl)phenoxy]-N-(2,6-difluorophenyl)-N-methyl-; or
a pharmaceutically acceptable salt thereof.

2. The compound of claim 1, wherein X¹ is NH.

3. The compound of claim 1, wherein X¹ is O.

4. The compound of claim 1, wherein Ar¹ is a six- to ten-membered heteroaryl substituted with R^{a} and further substituted with R^{b} and R^{c}.

5. The compound of claim 1, wherein Ar¹ is a six-membered heteroaryl substituted with R^{a} and further substituted with R^{b} and R^{c}.

6. The compound of claim 5, wherein Ar¹ is pyridinyl substituted with R^{a} and further substituted with R^{b} and R^{c}.

7. The compound of claim 5, wherein Ar¹ is pyridinyl substituted with R^{a}, where R^{a} is difluoromethyl or trifluoromethyl, and further substituted with R^{b} and R^{c}.

8. The compound of claim 5, wherein Ar¹ is pyridinyl substituted with R^{a}, where R^{a} is difluoromethyl or trifluoromethyl, and further substituted with R^{b} and/or R^{c}, where R^{b} is haloalkyl, alkoxy, halo, haloalkoxy, hydroxy, or cyano, and R^{c} is hydrogen, alkyl, halo, haloalkyl, alkoxy, haloalkoxy, hydroxy, cyano, cyanomethyl, aminocarbonylmethyl, heteroaryl, and heterocyclyl wherein said heteroaryl and heterocyclyl of R^{c} are unsubstituted or substituted with one, two, or three substituents independently selected from alkyl, halo, haloalkyl, and hydroxy.

9. The compound of claim 1, wherein Ar¹ is phenyl substituted with R^{a} and further substituted with R^{b} and R^{c}.

10. The compound of claim 9, wherein Ar¹ is phenyl substituted with R^{a}, where R^{a} is difluoromethyl or trifluoromethyl, and further substituted with R^{b} and R^{c}.

11. The compound of claim 9, wherein Ar¹ is phenyl substituted with R^{a} and R^{b} and/or R^{c}, where R^{a} is difluoromethyl or trifluoromethyl, R^{b} is haloalkyl, alkoxy, halo, haloalkoxy, hydroxy, or cyano, and R^{c} is hydrogen, alkyl, halo, haloalkyl, alkoxy, haloalkoxy, hydroxy, cyano, cyanomethyl, aminocarbonylmethyl, heteroaryl, and heterocyclyl, wherein said heteroaryl and heterocyclyl of R^{c} are unsubstituted or substituted with one, two, or three substituents independently selected from alkyl, halo, haloalkyl, and hydroxy.

12. The compound of claim 1, wherein R¹ is hydrogen, methyl, hydroxymethyl, 2-hydroxyethyl, 4-hydroxybenzyl, or aminocarbonylethyl.

13. The compound of claim 1, wherein R² is alkyl, cycloalkyl, or haloalkyl.

14. The compound of claim 1, wherein R¹ is hydrogen and R² is methyl, ethyl, isopropyl, cyclopropyl, or 2,2,2-trifluoroethyl.

15. The compound of claim 1, wherein Ar² is phenyl, wherein said phenyl is substituted with R^{g}, R^{h}, and Rⁱ independently selected from hydrogen, alkyl, cycloalkyl, cycloalkyloxy, halo, haloalkyl, alkoxy, haloalkoxy, hydroxy, cyano, and —CONH₂.

16. The compound of claim 1, wherein Ar² is phenyl substituted with R^{g}, R^{h}, and Rⁱ, wherein R^{g}, R^{h}, and Rⁱ are independently selected from hydrogen, —CONH₂, fluoro, chloro, bromo, cyano, methoxy, cyclopropyloxy, cyclobutyloxy, cyclopentyloxy, trifluoromethyl, or trifluoromethoxy.

17. The compound of claim 1, wherein Ar² is heteroaryl wherein said heteroaryl is substituted with R^{g}, R^{h}, and Rⁱ independently selected from hydrogen, alkyl, cycloalkyl, cycloalkyloxy, halo, haloalkyl, alkoxy, haloalkoxy, hydroxy, cyano, and —CONH₂.
